(19) United States
(12) Patent Application Publication
(10) Pub. No.: US 2023/0393140 A1
(43) Pub. Date: Dec. 7, 2023

(54) BINDING ASSAYS INVOLVING A PLURALITY OF SYNTHETIC COMPOUNDS, TARGETS, AND COUNTER TARGETS

(71) Applicant: SRI International, Menlo Park, CA (US)

(72) Inventors: Peter Madrid, San Jose, CA (US); Nathan Collins, San Mateo, CA (US); Michal Avital-Shmilovici, Sunnyvale, CA (US); Xiaohe Liu, Palo Alto, CA (US); Thomas Shaler, Fremont, CA (US)

(73) Assignee: SRI International, Menlo Park, CA (US)

(21) Appl. No.: 18/032,020

(22) PCT Filed: Oct. 15, 2021
(86) PCT No.: PCT/US2021/055226

§ 371 Date: Apr. 14, 2023

Related U.S. Application Data
(60) Provisional application No. 63/093,072, filed on Oct. 16, 2020.
(60) Provisional application No. 63/092,341, filed on Oct. 15, 2020.

(51) Int. Cl. G01N 33/68; G01N 33/543; G01N 33/551; G01N 33/58; G01N 21/64

(52) U.S. Cl. CPC G01N 33/6848; G01N 33/54386; G01N 2021/6439; G01N 33/582; G01N 21/6428; G01N 33/551

(57) ABSTRACT

An example binding assay includes a plurality of sub-regions, a plurality of synthetic compounds on beads, wherein each of the plurality of sub-regions includes one of the plurality of synthetic compounds, a biological target labeled with a first detectable label in each of the plurality sub-regions, and a biological counter target labeled with a second detectable label in each of the plurality of sub-regions. The biological counter target is configured to bind to the biological target when the biological target is in a first orientation. And, wherein a first subset of the plurality of synthetic compounds bind to the biological target and effect interactions between the biological target and the biological counter target.

### BACKGROUND

Polymer beads can be used to form libraries of compounds, such as naturally or synthetically produced oligomers or polymers (e.g., peptides, non-peptides and small molecules). In some instances, the concept of “one-bead one-compound” (OBOC) combinatorial libraries can be used to generate different compounds. The library can be used to screen for compounds that react to a target or provide a particular function. The OBOC library can include the use of a split synthesis approach. Using such an approach, libraries of compounds can be synthesized and screened for hits, such as a compound that provides a particular purpose (e.g., binding to a target). Structural determination of a screened compound can be performed by Edman chemistry, ladder sequencing, or isotope encoding. Structural determination techniques can have limitations, such as coding can interfere with the binding assay. For some targets, it can be beneficial to identify compounds that bind the target in an orientation that the target may be able and/or unable to bind to a counter target and/or that binds to multiple targets.

### SUMMARY

The present invention is directed to overcoming the above-mentioned challenges and others related to competitive binding assays, which may be used to screen libraries of synthetic compounds for compounds that bind to a biological target and that prevents or allows for binding of the biological target to a biological counter target.

Various embodiments of the present disclosure are directed to a competitive binding assay. The assay comprising a plurality of sub-regions, a plurality of synthetic compounds on beads, wherein each of the plurality of sub-region includes one of the plurality of synthetic compounds, a biological target labeled with a first detectable label in each of the plurality sub-regions, and a biological counter target labeled with a second detectable label in each of the plurality of sub-regions. Wherein the biological counter target is configured to bind to the biological target when the biological target is in a first orientation, and a first subset of the plurality of synthetic compounds bind to the biological target and effect interactions between the biological target and the biological counter target

In some aspects, the first subset of the plurality of synthetic compounds are to bind the biological target in a different orientation than the first orientation and inhibit interactions between the biological target and the biological counter target.

In some aspects, a second subset of the plurality of synthetic compounds bind to the biological target in the first orientation and a third subset of the plurality of synthetic compounds bind to the biological counter target.

In some aspects, the first subset of the plurality of synthetic compounds bind to the biological target such that the biological target is in the first orientation and permit for interactions between the biological target and the biological counter target.

In some aspects, the respective sub-regions associated with the first subset of the plurality of synthetic compounds provide a first fluorescent signal associated with the first detectable label and not a second fluorescent signal associated with the second detectable label.

In some aspects, the respective sub-regions associated with the first subset of the plurality of synthetic compounds provide a first fluorescent signal associated with the first detectable label and provide a second fluorescent signal associated with the second detectable label.

In some aspects, the plurality of synthetic compounds include: different subsets of a plurality of molecules, each of the plurality of molecules including a plurality of subgroups and exhibiting a mass spectrometry characteristic that is distinguishable from mass spectrometry characteristics of other molecules of the plurality; and cleavable groups linking at least some of the different subsets of the plurality of molecules and the bead to facilitate mass-spectroscopy based sequencing of the plurality of synthetic compounds.

In some aspects, the first detectable label is different from the second detectable label, and the biological target and biological counter target are proteins or nucleic acids.

Various embodiments are directed to an apparatus, comprising a binding assay and scanning circuitry. The binding assay comprising a plurality of sub-regions, a plurality of synthetic compounds on beads that are distinguishable by mass spectrometry, wherein each of the plurality of sub-regions includes one of the plurality of synthetic compounds, a biological target labeled with a first detectable label in each of the plurality of sub-regions, a biological counter target labeled with a second detectable label in each of the plurality of sub-regions. The biological counter target configured to bind to the biological target when the biological target is in a first orientation. The scanning circuitry is to (e.g., is configured to) identify a first subset of the plurality of synthetic compounds that bind to the biological target and that effects interactions between the biological target and the biological counter target.

In some aspects, the scanning circuitry is to (e.g., is configured to) provide a qualitative measure of binding affinity based a detected level of a signal associated with at least one of the first detectable label and the second detectable label.

In some aspects, the scanning circuitry is to (e.g., is configured to) provide a ratio of the biological target binding to the biological counter target binding based on detection of a first level of a signal associated with the first detectable label and a second level of a signal associated with the second detectable label.

In some aspects, the apparatus further includes cell picking circuitry to (e.g., is configured to) isolate the first subset of the plurality of synthetic compounds.

In some aspects, the plurality of synthetic compounds include: different subsets of a plurality of molecules, each of the plurality of molecules including a plurality of subgroups and exhibiting a mass spectrometry characteristic that is distinguishable from mass spectrometry characteristics of other molecules of the plurality; and cleavable groups linking at least some of the different subsets of the plurality of molecules and the bead.

In some aspects, the apparatus further includes mass spectrometry circuitry to (e.g., is configured to) sequence the first subset of the plurality of synthetic compounds by identifying the mass spectrometry characteristics of the respective subsets of molecules of the first subset of the plurality of synthetic compounds

In some aspects, the scanning circuitry is to (e.g., is configured to) identify the first subset of the plurality of synthetic compounds based on a first fluorescent signal associated with the first detectable label, and a second fluorescent signal associated with the second detectable label.

Some embodiments are directed to a method, comprising exposing a plurality of sub-regions of a binding assay to a biological target labeled with a first detectable label and a biological counter target labeled with a second detectable label, and detecting binding of a first subset of the plurality of synthetic compounds to the biological target that effects interactions between the biological target and the biological counter target by identifying signals associated with the first detectable label and the second detectable label. Wherein each of the plurality of sub-regions include one of a plurality of synthetic compounds on a bead that are distinguishable by mass spectrometry, wherein the biological counter target is configured to bind to the biological target when the biological target is in a first orientation.

In some aspects, detecting the binding of the first subset of the plurality of synthetic compounds includes identifying blocking of binding between the biological counter target and the biological target by identifying the signals associated with the second detectable label are below a threshold.

In some aspects, the method further includes isolating the first subset of the plurality of synthetic compounds and performing mass spectrometry to identify sequences of the first subset of the plurality of synthetic compounds.

In some aspects, exposing the assay to the biological target and the biological counter target includes incubating the plurality of synthetic compounds with the biological target and the biological counter target, and, after incubating, washing unbound biological targets and biological counter targets from the assay.

In some aspects, the plurality of synthetic compounds include: different subsets of a plurality of molecules, each of the plurality of molecules including a plurality of subgroups and exhibiting a mass spectrometry characteristic that is distinguishable from mass spectrometry characteristics of other molecules of the plurality; and cleavable groups linking at least some of the different subsets of the plurality of molecules and the bead. And, the method further includes separating the first subset of the plurality of synthetic compounds from the beads and the respective different subset of molecules forming the synthetic compounds of the first subset from one another, and sequencing the first subset of the plurality of synthetic compounds by identifying the mass spectrometry characteristics of the respective different subset of molecules via mass spectrometry.

Other embodiments are directed to various synthetic compounds as further described herein.

### BRIEF DESCRIPTION OF THE DRAWINGS

Various example embodiments can be more completely understood in consideration of the following detailed description in connection with the accompanying drawings, in which:

FIG. 1 illustrates an example binding assay for a biological target and a biological counter target, in accordance with the present disclosure.

FIGS. 2A-2B illustrate an example of a plurality of synthetic compounds forming part of a binding assay, in accordance with the present disclosure.

FIGS. 3A-3C illustrate example binding assays and different binding outputs, in accordance with the present disclosure.

FIGS. 4A-4B illustrate example methods of screening a plurality of synthetic compounds for binding to a biological target that effects binding to a biological counter target using a binding assay, in accordance with the present disclosure.

FIG. 5 illustrates an example process for screening a library of a plurality of synthetic compounds for binding to a biological target using a binding assay, in accordance with various embodiments.

FIG. 6 illustrates an example of a system used to screen a library of a plurality of synthetic compounds, in accordance with the present disclosure.

FIG. 7 illustrates an example system used to screen a library of a plurality of synthetic compounds, in accordance with the present disclosure.

FIGS. 8A-8D illustrate example screening of a library of a plurality of synthetic compounds, in accordance with the present disclosure.

FIGS. 9A-9B illustrate examples of designed and validated libraries of synthetic compounds, in accordance with the present disclosure.

FIGS. 10A-10D illustrate example results from a binding assay, in accordance with the present disclosure.

FIGS. 11A-11F illustrate example biological relevance and stability of non-natural polymers in biological matrices, in accordance with the present disclosure.

FIG. 12 illustrates an example detection rate, in accordance with the present disclosure.

FIGS. 13A-13B illustrate example distribution of amino acids in libraries, in accordance with the present disclosure.

FIGS. 14A-14L illustrate example mass spectrometry results of hits from screening an assay, in accordance with the present disclosure.

FIG. 15 illustrates an example experiment for K-Ras and Raf protein-protein interaction (PPI) profiling, in accordance with the present disclosure.

FIGS. 16A-16C illustrate example PPI profiling data for synthetic compounds, in accordance with the present disclosure.

### DETAILED DESCRIPTION

In the following detailed description, reference is made to the accompanying drawings which form a part hereof, and in which is shown by way of illustration specific examples in which the disclosure can be practiced. It is to be understood that other examples can be utilized, and various changes may be made without departing from the scope of the disclosure. The following detailed description, therefore, is not to be taken in a limiting sense, and the scope of the disclosure is defined by the appended claims. It is to be understood that features of the various examples described herein may be combined, in part or whole, with each other, unless specifically noted otherwise.

Natural biological polymers, such as peptides, proteins, and nucleic acids, have evolved molecular recognition functionalities to produce highly specific binding interactions and catalytic enzymatic functions. There is growing interest in synthetic compound discovery, such as polymers or oligomers, using chemical synthesis to further access molecular diversity in novel three-dimensional folded structures with related functionality as affinity reagents, therapeutics, and catalysts, among other functionalities. In some aspects, a library of synthetic compounds is used for screening for reaction or interaction of the compounds to a plurality of biological targets, generate a diagnostic assay, and/or identify new compounds that provide specific functionality. The library includes a plurality of polymer beads, each bead having a different synthetic compound attached thereto. Each synthetic compound is formed of a plurality of different molecules that have known mass spectrometry characteristics and which are distinguishable (e.g., unique) relative to mass spectrometry characteristics of the other molecules in the plurality, the molecules sometimes herein being referred to as a “ptych”. Distinguishable or unique mass spectrometry characteristics, as used herein, includes or refers to a value of a mass spectrometry characteristic that is separable by a threshold from other values in the set of molecules and/or subgroups. Respective compounds that are hits (e.g., bind to at least one of the plurality of targets) are identified via mass spectrometry characteristics of the molecules that form the synthetic compound, such that the synthetic compounds can be used for diagnosis, treatment, or other purposes.

Example embodiments of the present disclosure are directed to binding assays, systems for screening a binding assay, and methods of screening a binding assay for synthetic compounds that bind to a biological target in a manner that effects (e.g., allows, mitigates, or prevents) binding between the biological target and a biological counter target. The plurality synthetic compounds of the assay are exposed to each of the biological target labeled with a first detectable signal and the biological counter target labeled with a second detectable signal. Depending on the type of binding assay, the detectable signals are used to identify hits having the first detectable signal and not the second detectable signal or both the first detectable signal and the second detectable signal. The mass spectrometry characteristics of the molecules of the synthetic compounds that are hits act as barcodes that are readout by performing mass spectrometry. For example, the molecules used to form the compounds in the library each have different (and known) molecular masses, fragmentation patterns, elution times and/or isotope distributions that can be identified using mass spectrometry. The respective mass spectrometry characteristics can map to, for example, via a map, key, or other association, a respective molecule and each molecule is assigned a position in the sequence of compounds formed in the library. As is further described herein, the molecules are formed of a plurality of subgroups, such as naturally occurring and non-naturally occurring amino acids, among other types of monomers and functional groups. Each subgroup additionally has a distinguishable mass spectrometry characteristic and is assigned a position in the sequence of molecules of the library. The mass spectrometry characteristic thereby identifies the molecule itself, the position of the molecule in the sequence of the synthetic compound, as well as the sequence of subgroups forming the molecule.

In some embodiments, the reacted bead can be used to identify the respective synthetic compound by separating the synthetic compound from the bead and the molecules from one another via cleavage and performing mass spectrometry. Mass spectrometry characteristics identified are compared to possible or known mass spectrometry characteristics of possible molecules in the library and are used to identify the sequence of the synthetic compound (e.g., the order of the plurality of molecules forming the compound which includes identification of the sequence of subgroups forming each molecule). Libraries formed in accordance with the present disclosure can be on an order of 10\hat{ }8 to 10\hat{ }9 or more compounds and can include beads that are sub-ninety microns in diameter, such as ten microns in diameter. The library can be screened using an optical scanner, as further described herein.

Some embodiments are directed to a binding assay, a system including a binding assay, and/or a method of screening a plurality of synthetic compounds using a binding assay. The binding assay includes a plurality of sub-regions, a plurality of synthetic compounds on beads, wherein each of the plurality of sub-regions includes one of the plurality of synthetic compounds, a biological targets in each of the plurality of sub-regions, and a biological counter target in each of the plurality of sub-regions. The biological target is labeled with a first detectable label and the biological counter target is labeled with a second detectable label that is distinguishable from the first. The biological counter target can be configured to bind to the biological target when the biological target is in a first orientation. A first subset of the plurality of synthetic compounds can bind to the biological target in a manner that effects interactions between the biological target and the biological counter target. An effect on the interactions can include inhibiting, mitigating, allowing, or increasing binding between the biological counter target and the biological target, in various embodiments. The binding assay can be analyzed to identify the first subset of the plurality of synthetic compounds and at least some of the first subset of the plurality of synthetic compounds can be selected, isolated (e.g., picked), and analyzed for sequencing using the mass spectrometry characteristics.

In some embodiments, the binding assay includes a competitive binding assay. In such embodiments, the first subset of the plurality of synthetic compounds can bind to the biological target, such that the biological target is in a different orientation than the first orientation and which inhibits interactions between the biological target and the biological counter target. In such embodiments, the respective sub-regions of the binding assay that are associated with the first sub-set of the plurality of synthetic compounds can provide a first signal associated with the first detectable label and not provide a second signal associated with the second detectable label.

In some embodiments, the binding assay includes an association binding assay. For example, the first subset of the plurality of synthetic compounds can bind to the biological target in the first orientation and allow for interactions between the first biological target and the biological counter target. In such embodiments, the respective sub-regions of the binding assay that are associated with the first sub-set of the plurality of synthetic compounds can provide a first signal associated with the first detectable label and provide a second signal associated with the second detectable label.

Although the above describes use of binding assays including a biological counter target, embodiments are not so limited and can be directed to an assay used to detect binding to a biological target without the use of a biological counter target. In some embodiments, the binding assay can include two or more biological targets which are screened to identify synthetic compounds that bind to both the first and second (or more) biological targets, such as binding to different regions of the synthetic compound. The respective sub-regions of the binding assay associated with the first sub-set of the plurality of synthetic compounds can provide a first signal associated with the first detectable label and provide a second signal associated with the second detectable label.

Furthermore, although the above describes binding assays involving multiple targets, embodiments are not so limited and can include a variety of different types of assays. Various embodiments are directed to synthetic compounds which bind to particular biological targets, and which can be identified using a competitive binding assay, an association binding assay, or other types of binding assays including or associated with a library of synthetic compounds. In some embodiments, the synthetic compounds can have affinity to one of five biological targets of biomedical interest and which have affinities in the nanomolar to sub-nanomolar range. In some embodiments, the synthetic targets can inhibit protein-protein interactions (PPIs) and protein-glycan interactions and have exceptional biological activity and stability.

Various embodiments are directed to a synthetic compound comprising: N plurality of molecules, each of the N plurality of molecules being formed of M amino acids, wherein N is between six and nine and M is between three and five, and cleavable groups positioned between at least some of the N plurality of molecules. The synthetic compound is configured to bind to a biological target selected from a group consisting of: K-Ras, interleukin 6 (IL-6), tumor necrosis factor alpha (TNFα), IL-6 receptor (IL-6R), and asialoglycoprotein receptor 1 (ASGPR). Libraries of synthetic compounds are not limited to the synthetic compounds wherein N is between six and nine and M is between three and five. In some embodiments, the library of synthetic compounds can include N between two and fifty, and M between two and ten.

IL-6, TNFa, and IL-6R are cytokines and a cytokine receptor involved in immunological signaling processes and are therapeutic targets for antibody-based therapeutics. K-Ras is an oncology drug target that is mutated in around thirty percent (%) of cancers leading to uncontrolled cell proliferation, particularly in cancers with poor prognosis such as pancreatic and lung cancers. ASGPR is a glycoprotein receptor that is predominantly found on the surface of liver hepatocytes and can be utilized as a mediator of liver-specific intracellular drug delivery of nucleic acid based therapeutics.

Disruption of K-Ras binding to its singling partner can involve a protein-protein interaction inhibitor and ASGPR is a C-type lectin glycan receptor, both of which are challenging targets for traditional small-molecule approaches and therefore represent interesting molecular targets for synthetic compounds, e.g., non-natural polymer ligands. These ligands can be used as therapeutics or a diagnostic/detection reagents. Current treatment for indications associated with IL-6, IL-6R and TNFα is mostly based on antibody drugs. Antibodies have great therapeutic properties but suffer from several limitations, particularly biological and thermal stability, and require cold chain and special storage. In addition, it takes decades to discover, develop and manufacture therapeutic antibodies (Abs). There is not currently a drug that can regulate K-Ras intracellular-activity, and therefore cancer types that involve K-Ras mutants can be difficult to treat due to lack of available cures and/or treatments. ASGPR can currently be targeted by sugar molecules only. Embodiments in accordance with the present disclosure includes synthetic compounds which are specific to one of K-Ras, ASGPR, IL-6, IL-6R and TNFα.

In some embodiments, the above-described library can be screened to identify various functions, such as drugs, reagents, sensors or materials. The synthetic compounds, which can include polymers or oligomers, are formed of a plurality of cleavable fragments, which are referred to herein as “molecules” and sometimes herein interchangeably referred to as “ptychs”. The cleavable fragments have mass spectrometry characteristics that define the sequence of molecules forming the synthetic compound. The library can be formed by using logic circuitry to design molecules (e.g., the cleavable fragments) formed of different subgroups, analyzing the plurality of molecules to determine their mass spectrometry characteristics, selecting at least some of the molecules to use in the library based on their mass spectrometry characteristics, and assigning each of the selected molecules to a position in the sequence of synthetic compounds in the library. The molecules can be correlated with the assigned positions in a memory circuit of the logic circuitry. The library can be formed by synthesizing a plurality of synthesized synthetic compounds using the selected molecules and based on assigned positions and/or by defining each of the plurality of synthetic compounds as a data object stored in the memory circuit using the selected molecules and the assigned positions. The molecules can be selected to ensure that all of the mass spectrometry characteristics of the selected molecules are distinguishable from other selected molecules, such as their molecular weights being at least five parts-per-million (ppm) apart. Although embodiments are not so limited and can include different thresholds that separate the mass spectrometry characteristics. After selecting the molecules to use and assigning respective positions, the synthetic compounds can be synthesized using a combination of mix-and-split synthesis to synthesize the compounds on beads that are composed of multiple molecules with cleavable groups linking at least some of the multiple molecules and/or linking a respective one of molecules to the bead. The cleavable groups can be inserted into the backbone of the synthetic compound and positioned between each molecule (e.g., ptych) at each mix-and-split step in the synthesis.

The library can be screened for hits to a target, such as using an optical scanner. An example of an optical scanner is a fiber optic scanner, which includes a fiber optic bundle array, a laser, and imaging circuitry (e.g., camera), such as Fiber-optic Array Scanning Technology (FAST). The FAST technology is based on the concept of “copying” a plate containing beads with a scanning laser and collecting a high resolution capture image of the plate using a densely packed fiber optic array bundle. The FAST system can allow for rapid scanning at speeds of between 1 million and 25 million cells per minute. For more specific and general information regarding an example FAST system, reference is made to Hsieh H B, Marrinucci D, Bethel K, et al., “High speed detection of circulating tumor cells”, Biosensors and Bioelectronics, 2006; 21: 1893-1899, and Krivacic R T, Ladanyi A, Curry D N, et al., “A rare-cell detector for cancer”, Proc Natl Acad Sci USA, 2004; 101: 10501-10504, and US Patent Application, Pub. No US2021/0208157, entitled “Affinity Reagent and Catalyst Discovery Through Fiber-Optic Array Scanning Technology”, each of which are fully incorporated herein by reference.

An assay can be performed with the beads to identify synthetic compounds exhibiting a particular function, which may or may not include competitive or associative binding. In specific embodiments, the assay can be used to bind to a protein, inhibit an enzyme, and neutralize or kill a cell, among other functions. The assay can be screened to identify a synthetic compound that exhibits a particular function and the identified compound can be isolated. For example, the detected activity is assessed via a fluorescent readout of the assay using an optical scanner. Identified beads that are suspected of exhibiting the particular function or activity are identified based on the scan and removed from the screening plate and placed in wells or tubes using the selection circuitry. Removed beads are further processed to release the synthetic compound on the bead and separate the molecules used to form the compound via cleavage, as previously described. The respective mass spectrometry characteristics of the molecules present map to, for example, via a map, key, or other association, the molecule and a position in the sequence of the synthetic compound, which is used to sequence the synthetic compound.

In various embodiments, the plurality of synthetic compounds can be designed, formed, and/or include at least some of substantially the same features and/or attributes as described by U.S. Pat. No. 10,882,020, entitled “Method for Topology Segregation for Polymer Beads”; and US Patent Application, Pub. No. US2021/0065848, entitled “Mass Spectrometry Distinguishable Synthetic Compounds, Libraries, and Methods Thereof”, each of which are fully incorporated herein by reference in their entirety for their teachings.

As may be appreciated and as used herein, a polymer bead or bead includes or refers to a polymer material formed in a three-dimensional shape, such as a sphere, an ellipsoid, oblate spheroid, and prolate spheroid shapes. A synthetic compound includes or refers to a non-natural oligomer or polymer formed in a library that is used to test for different functionalities, such as binding to a biological target, not binding to a biological counter target, neutralizing or killing a biological target, and/or providing physical properties, among other functionalities. A library of synthetic compounds, as described above, includes or refers to a plurality of synthetic compounds, which can be physical chemicals that are synthesized and used to screen for various functionality. In some specific embodiments, the physical chemicals are each connected to polymer bead. Accordingly, in some specific embodiments, a library of synthetic compounds includes a plurality of different polymer beads, each bead coupled to a different physically-made synthetic compounds.

Molecule, sometimes referred to as a “ptych”, includes and/or refers to a set of subgroups (e.g., amino acids and other monomers and/or functional groups) bonded together. As described herein, the molecules are distinguishable from one another via mass spectrometry characteristics and can be formed of a plurality of monomers, such as amino acids. A subgroup includes and/or refers to a monomer or functional group. A functional group includes or refers to a group of atoms and/or bonds within a molecule (e.g., the polymer bead) that are responsible for a characteristic chemical reaction of the molecule. In some embodiments, the molecules include a plurality of amino acids, among other functional groups. The amino acids can include natural and non-natural amino acids, and which can include standard scaffold of α-amino acids. In some embodiments, each molecule includes at least one non-natural amino acid (or another non-natural functional group). A cleavable group includes and/or refers to a functional group that cleaves to chemically separate molecules from one another and/or the compound from the bead. In various embodiments, the cleavable group can form part of and/or links (e.g., connects) respective ones of the plurality of molecules and one of the plurality of molecules to the bead. Mass spectrometry characteristics include and/or refer to properties or values observed from performing mass spectrometry on a compound, molecule, and/or mixture of molecules. Example mass spectrometry characteristics include molecular weighs, fragmentation patterns, elution times, isotope distributions, and chromatographic retention times (e.g., chromatographic retention isotope distribution).

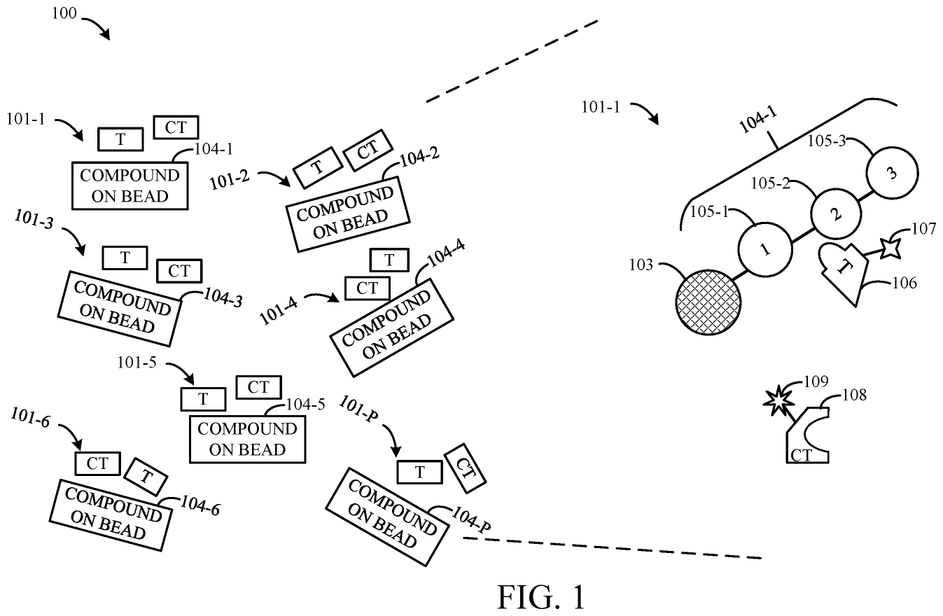

Turning now to the figures, FIG. 1 illustrates an example binding assay for a biological target and a biological counter target, in accordance with the present disclosure.

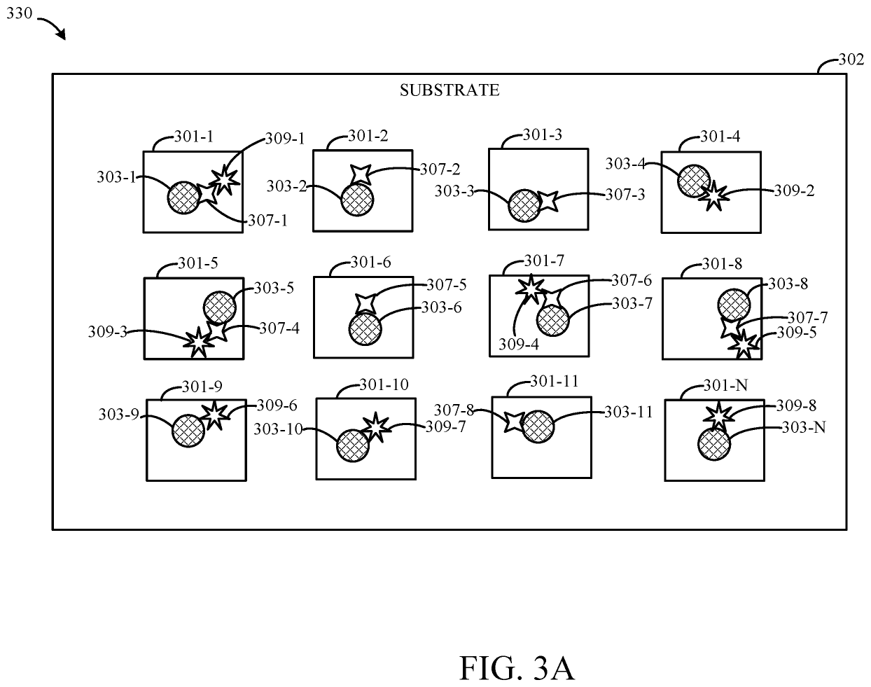

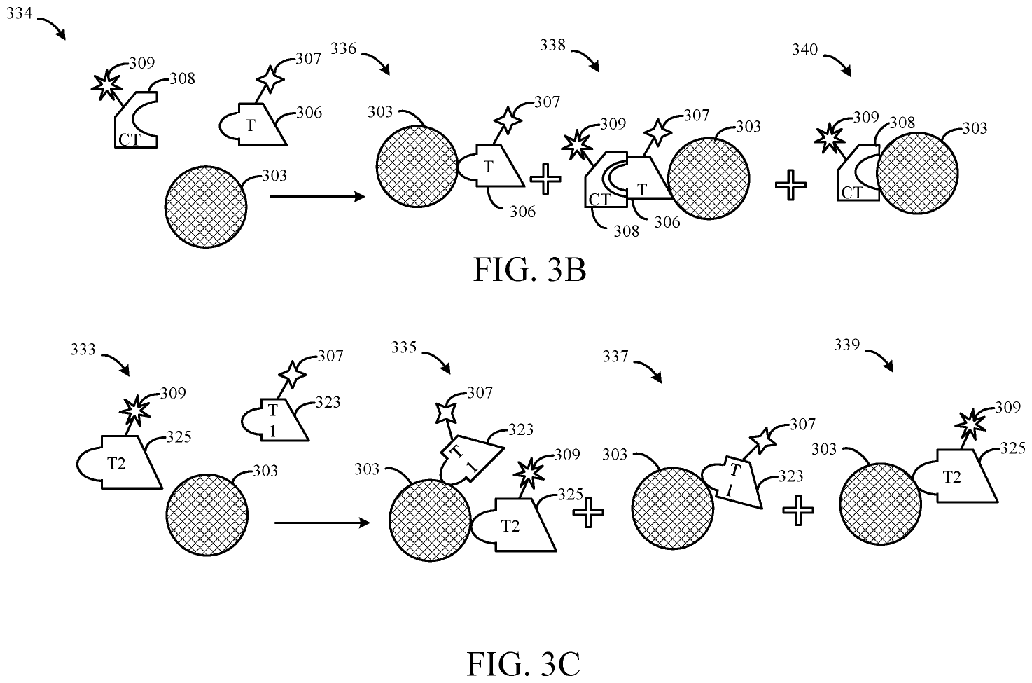

The binding assay 100 includes a plurality of sub-regions 101-1, 101-2, 101-3, 101-4, 101-5, 101-6, 101-P (herein generally referred to as the “sub-regions 101” for ease of reference). The sub-regions 101 can include or form part of a substrate, as further illustrated herein by FIG. 3A. The substrate and/or plurality of sub-regions 101 can be formed a variety of materials and be configured in a variety of arrangements. In some embodiments, the substrate includes a glass slide or other type of screening plate. In some embodiments, the sub-regions 101 can be separable and distinct from one another and/or can include one of the plurality of synthetic compounds 104-1, 104-2, 104-3, 104-4, 104-5, 104-6, 104-P (herein generally referred to as “the plurality of synthetic compounds 104), such as a plate with a plurality of wells. In some embodiments, the sub-regions 101 can include different portions of glass plate or slide, which may not include separations from one another and may include one or multiple of the plurality of synthetic compounds 104.

The binding assay 100 further includes the plurality of synthetic compounds 104 on beads. As noted above, each of the plurality of sub-regions 101 can include (at least) one of the plurality of synthetic compounds 104. As previously described, the plurality of synthetic compounds 104 can include non-natural polymers. In various embodiments, the plurality of synthetic compounds 104 include different subsets of a plurality of molecules and cleavable groups linking at least some of the different subsets of the plurality of molecules and one molecule of each of the different subsets of the plurality of molecules to the beads to facilitate mass-spectroscopy based sequencing of the plurality of synthetic compounds. Each of the plurality of molecules include a plurality of subgroups, such as natural and non-natural amino acids, and exhibit a mass spectrometry characteristic that is distinguishable from mass spectrometry characteristics of other molecules of the plurality. In some embodiments, each of the plurality of molecules include a plurality of amino acids, including at least one non-natural amino acid, and optionally include other functional groups.

The binding assay 100 further includes a biological target in each of the plurality of sub-regions 101 and a biological counter target in each of the plurality of sub-regions 101, as illustrated by the plurality of “T” and “CT” and further illustrated by the particular biological target 106 and biological counter target 108 in the close-up view of the first sub-region 101-1. The biological target is labeled with a first detectable label, as illustrated by the biological target and the first detectable label 107, and the biological counter target is labeled with a second detectable label, as illustrated by the particular biological counter target 108 and the second detectable label 109. In some examples, the first and second detectable labels 107, 109 can include different fluorescent labels. However, embodiments are not so limited and other types of labels can be used, such as radioactive isotopic labels, electrical signals labels, other types of optical labeling (e.g., colorants, biotins), reporter enzymes, among other types of labels.

Referring to the close-up view of region 101-1, the counter biological target 108 can be configured to bind to the biological target 106 when the biological target 106 is in a particular orientation, sometimes herein referred to as a first orientation. The orientation of the biological target 106 can be dependent on if and/or how the biological target 106 is bound to another component, such as directional binding of the biological target 106 to the other component. In some embodiments, directional binding of the biological target 106 can leave a binding region of the biological target 106 accessible to the biological counter target 108 to which the biological counter target 108 has an affinity for. As may be appreciated, when the biological target 106 is unbound, the binding region is accessible. When the biological target 106 is bound to a synthetic compound, such as 104-1, in a different orientation than the first orientation, the binding region of biological target 106 that the biological counter target 108 is configured to bind to can be inhibited or blocked such that the biological counter target 108 cannot bind or has reduced affinity to the biological target 106. In contrast, if the biological target 106 is bound to the synthetic compound in the first orientation, the binding region of biological target 106 that the biological counter target 108 is configured to bind to is accessible to the biological counter target 108, such that the biological counter target 108 binds to the biological target 106.

As used herein, a biological target includes and/or refers to a component of interest associated with a living organism (e.g., a biological component of interest), which are screened against the synthetic compounds 104 to identify respective compounds that bind to the biological target and where a “plurality of the biological targets” or a “plurality of the biological target” includes a plurality of copies of the same biological target. A biological counter target includes and/or refers to a component of interest associated with a living organism, which may bind to the biological target, and where a “plurality of the biological counter targets” or a “plurality of the biological counter target” includes a plurality of copies of the same biological counter target. Example targets and counter targets can include proteins, glycans, antibodies, antigens, nucleic acid segments (e.g., DNA, RNA), among others.

In some embodiments, the biological target 106 and biological counter target 108 are both screened against the plurality of synthetic compounds 104 on beads to identify respective synthetic compounds that bind to the biological target 106 in a manner that effects (e.g., allows for or inhibits) interactions between the biological target 106 and the biological counter target 108, such as binding the biological target 106 in the first orientation or in a different orientation than the first orientation.

In some embodiments, the binding assay 100 includes a competitive binding assay. Competitive binding refers to competing of binding to the biological target 106 by the biological counter target 108 and the synthetic compounds 104. In some embodiments, the first subset of the plurality of synthetic compounds 104 can bind the biological target 106 in a manner that inhibits interactions between the biological target 106 and the biological counter target 108. For example, each of the first subset of the plurality of synthetic compounds 104 can directionally bind to the biological target 106 such that the binding region of the biological target 106, that the biological counter target 108 has an affinity for, is blocked.

For example, a first subset of the plurality of synthetic compounds 104 can bind to the biological target 106, such that the biological target 106 is in a different orientation than the first orientation and inhibits interactions between biological target 106 and the biological counter target 108. As noted above, each sub-region includes a biological target such that the binding assay 100 includes a plurality of the biological target. Each of the first subset of the plurality of synthetic compounds 104 can directionally bind to one of a first subset of the plurality of biological targets such each of the first subset of the plurality of first biological targets are bound in a different orientation than the first orientation and the binding region of the biological targets, that the plurality of biological counter target have an affinity for, are blocked (e.g., partially or wholly).

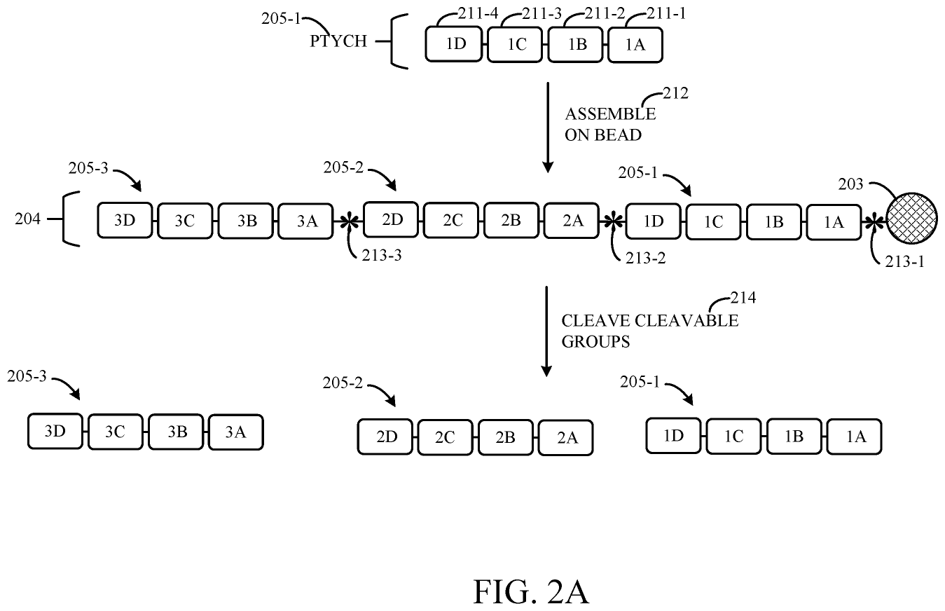

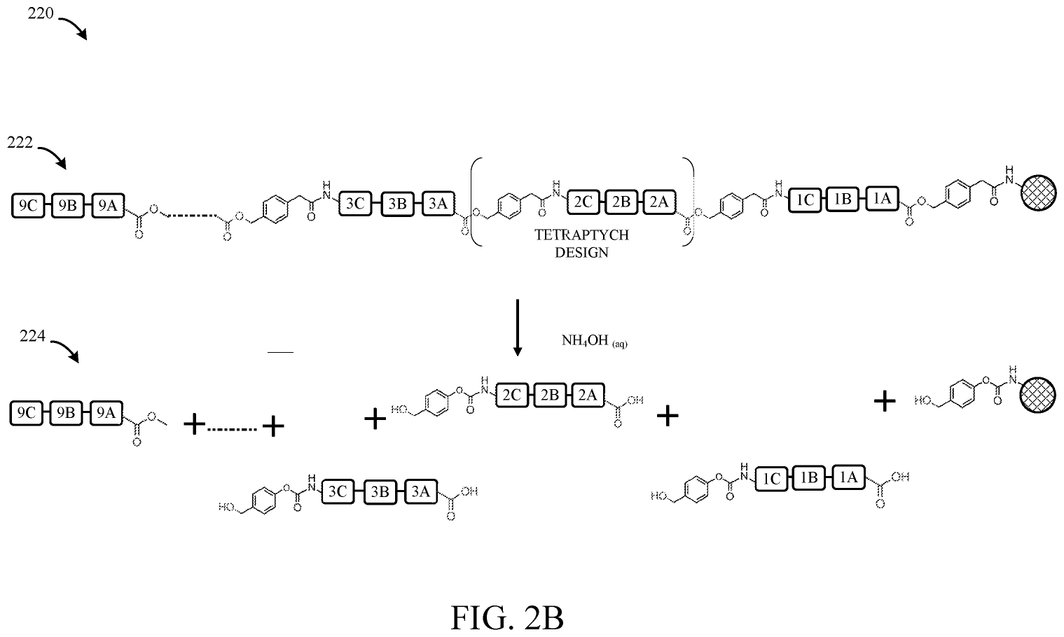

The close-up view 101 of FIG. 1 shows an example of one region 101-1 of the plurality of sub-regions. As shown by the close-up view, the region 101-1 includes a synthetic compound 104-1 of the plurality of synthetic compounds 104 bound to a bead 103 and which is formed of a plurality of molecules 105-1, 105-2, 105-3 with cleavable groups linking the plurality of molecules 105-1, 105-2, 105-3, as further illustrated by FIG. 2A. Referring to back to FIG. 1, each of the plurality of molecules 105-1, 105-2, 105-3 can be formed of and/or include a plurality of amino acids (e.g., natural and/or non-natural amino acids) and/or other subgroups. The synthetic compound 104-1 binds to the biological target 106 such that the biological target 106 is in a different orientation than the first orientation and which inhibits interactions between the biological target 106 and the biological counter target 108. As shown, the biological target 106 is labeled by a first detectable label 107 and the biological counter target 108 is labeled by a second detectable label 109. The sub-region 101-1 associated with the bead 103 can exhibit the first detectable label 107 but not the second detectable label 109 and can be identified, selected, isolated, and/or further analyzed, as further described herein. Accordingly, respective sub-regions of the plurality of sub-regions 101 associated with the first subset of the plurality of synthetic compounds 104 can provide a first signal associated with the first detectable label 107 and not provide a second signal associated with the second detectable label 109, which is used to identify the first subset of the plurality of synthetic compounds 104 for a competitive binding assay.

As further illustrated herein by FIG. 3B, the assay 100 can further include a second subset of the plurality of synthetic compounds 104 that bind to the biological target 106 (e.g., a second subset of the plurality of biological targets), such that the biological target 106 (e.g., the second subset of the plurality of biological targets) is in the first orientation and bind to the biological counter target 108 (e.g., to a first subset of the plurality of biological counter targets). The assay 100 can further include a third subset of the plurality of synthetic compounds 104 that bind to the biological counter target 108 (e.g., a second subset of the plurality of biological counter target) and not the biological target 106. The sub-regions 101 associated with the second subset of the plurality of synthetic compounds 104 can exhibit the first detectable label 107 and the second detectable label 109. The sub-regions 101 associated with the third subset of the plurality of synthetic compounds 104 can exhibit the second detectable label 109 and not the first detectable label 107. A fourth subset of the plurality of synthetic compounds 104 can bind to neither of the biological target 106 and the biological counter target 108, the associated sub-regions 101 exhibit neither of the first detectable label 107 and the second detectable label 109.

Competitive binding assays can be used to identify synthetic compounds that bind the biological target 106 and additionally inhibit interactions between the biological target 106 and the biological counter target 108, such as inhibiting protein-protein interactions (PP), protein-glycan interactions, among others. With a competitive binding assay, sub-regions associated with the first subset of the plurality of sub-regions 101 can exhibit or provide the first detectable signal associated with the first detectable label 107 and not the second detectable signal associated with the second detectable label 109.

Embodiments are not limited to competitive binding assays. In some embodiments, the binding assay 100 includes an association binding assay that includes the biological target 106 and the biological counter target 108. In some embodiments, the first subset of the plurality of synthetic compounds 104 can bind to the biological target 106 such that the biological target 106 is in the first orientation and which allows for interactions between the biological target 106 and the biological counter target 108. With an associative binding assay, sub-regions associated with the first subset of the plurality of sub-regions 101 can exhibit or provide the first detectable signal associated with the first detectable label 107 and the second detectable signal associated with the second detectable label 109.

In some embodiments, prior to analyzing the results of the binding assay 100, unbound biological targets and unbound biological counter targets can be washed away. However, embodiments are not so limited.

In various embodiments, the first detectable label 107 is different from the second detectable label 109, such that the labels 107, 109 are distinguishable from one another. In some embodiments, when both the first detectable label 107 and the second detectable label 109, a signal can be detected that includes a combination of each.

Although the above describes binding assays including a biological target and a biological counter target, embodiments are not so limited. In some embodiments, the binding assay 100 includes two or more biological targets which are screened to identify synthetic compounds that bind to both the first and second biological targets, such as binding to different regions of the synthetic compound. In some embodiments, the respective sub-regions of the binding assay 100 that are associated with the first sub-set of the plurality of synthetic compounds 104 can provide a first signal associated with the first detectable label and provide a second signal associated with the second detectable label which are indicative of the presence of the first and second biological targets.

FIGS. 2A-2B illustrate an example of a plurality of synthetic compounds forming part of a binding assay, in accordance with the present disclosure. As previously described, the synthetic compounds are formed of molecules, sometimes referred to as a “ptych”, which includes or refers to a set of monomers bonded together. Cleavable groups can link respective monomers and link one of the monomers and the bead.

FIG. 2A depicts an example “tetraptych” (e.g., “four” and ptysso, i.e., “to fold”). A tetraptych 205-1 is defined as a set of four subgroups 211-1, 211-2, 211-3, 211-4 (e.g., amino acids) with folding properties that make up one molecule (e.g., the tetraptych 205-1) in a longer sequence. The full sequence of the synthetic compound 204 can then be formed by linking multiple ptychs 205-1, 205-2, 205-3 together with cleavable groups 213-1, 213-2, 213-3 and linking a respective ptych and the bead 203, at 212 (e.g., linking the bead 203 and the first ptych 205-1, linking the first ptych 205-1 and the second ptych 205-2, and linking the second ptych 205-2 and the third ptych 205-3). Each tetraptych 205-1, 205-2, 205-3 is composed of monomers that enable diversity of physiochemical and structural properties at the individual ptych level. With this approach, polymer library scale and diversity can be built by choosing the number of ptychs in a linear sequence and the number of ptych variants at each position. The size of the ptych is also flexible. For example, two monomers define a diptych; four monomers define tetraptychs; and six monomers define hexaptychs, and so forth.

By connecting ptychs via chemically cleavable groups 213-1, 213-2, 213-3 that can be cleaved under orthogonal conditions used in library synthesis and screening, such as illustrated at 214, the sequence of a synthetic compound 204 can be directly read by mass spectrometry (MS). FIG. 2A depicts a general polymer design with three tetraptychs 205-1, 205-2, 205-3, each consisting of four diversity-building block monomers and a cleavable group 213-1, 213-2, 213-3. Cleavage of the cleavable group 213-1, 213-2, 213-3 yields an equimolar mixture of the three tetraptychs 205-1, 205-2, 205-3. In some embodiments, the phenyl-acetamido-methylene (PAM) linker is used as the cleavage linker. PAM can provide an ester bond between ptychs that is stable to the Fmoc/tBu/Alloc protection strategy to build the polymers, but it can be readily cleaved using aqueous base such as ammonium hydroxide or sodium hydroxide.

FIG. 2B depicts a general design 220 with multiple tetraptychs, as shown at 222, each consisting of a PAM linker and three diversity building-block monomers. Hydrolysis of the esters yields a mixture of the different tetraptychs, as shown at 224. The ptych design 220 allows for building blocks to be selected so each ptych diversity element in a sequence has a unique molecular weight. As a result, each of the ptychs present in the mixture after cleavage can be identified by its mass using high-resolution LC-MS and an electrospray source on an LTQ-Orbitrap XL mass spectrometer that can detect molecular ions at the femtomole level. This provides three orders of magnitude more sensitivity than the MS fragmentation methods used in peptide and protein sequencing. Sequencing of ptych designed libraries is independent of the nature of the building blocks and virtually any chemical building block can be incorporated, whereas MS fragmentation sequencing is extremely dependent on the nature of the fragmentation patterns of the backbone chemical bonds of the building blocks and has largely been limited to α—amino acid peptide polymers.

FIGS. 3A-3C illustrate an example binding assay and different binding outputs, in accordance with the present disclosure. FIG. 3A illustrates an example competitive binding assay 330, which can include an implementation of and/or substantially the same features of the binding assay 100 of FIG. 1, including a plurality of synthetic compounds on beads, as illustrated by the beads 303-1, 303-2, 303-3, 303-4, 303-5, 303-6, 303-7, 303-8, 303-9, 303-10, 303-N (herein generally referred to as the “synthetic compounds on beads 303”), a plurality of biological targets labeled by a first detectable label 307-1, 307-2, 307-3, 307-4, 307-5, 307-6, 307-7, 307-8 (herein generally referred to as “biological targets labeled by the first detectable label 307”), and a plurality of biological counter targets labeled by a second detectable label 309-1, 309-2, 309-3, 309-4, 309-5, 309-6, 309-7, 309-8 (herein generally referred to as “biological counter targets labeled by the second detectable label 309”). For ease of reference, FIG. 3A only shows the beads 303 and labels 307, 309, which are representative of the synthetic compounds, biological targets, and biological counter targets which are respective bound thereto.

In some embodiments, a substrate 302 can include a plurality of sub-regions 301-1, 301-2, 301-3, 301-4, 301-5, 301-6, 301-7, 301-8, 301-9, 301-10, 301-11, 301-N (herein generally referred to as the “sub-regions 301”). The sub-regions 301 can be independent and separate from one another, such as wells of a well plate. In other embodiments, the sub-regions 301 can include different geographic regions or portions of the substrates 301 which can abut one another, such as areas of a glass slide. In some embodiments, each sub-region 301 can include one of the synthetic compounds on beads 303, and in other embodiments, can include more than one.

As previously described, the synthetic compounds on beads 303 can be exposed to the biological target labeled with the first detectable label 307 and the biological counter target labeled with the second fluorescent label 309. In response, the binding assay 330 can be analyzed to identify respective synthetic compounds that bind the biological target in an orientation, sometimes herein referred to as a different orientation than the first orientation, that inhibits interactions between the biological target and the biological counter target.

As shown by FIG. 3A, a first subset of the plurality of synthetic compounds, represented by beads 303-2, 303-3, 303-6, 303-11, bind to a first subset of the plurality of biological targets, represented by first detectable labels 307-2, 307-3, 307-5, 307-8. A second subset of the synthetic compounds, represented by beads 303-1, 303-5, 303-7, 303-8, bind to a second subset of the plurality of biological targets, represented by first detectable labels 307-1, 307-4, 307-6, 307-7, and which bind to a first subset of the plurality biological counter targets, represented by second detectable labels 309-1, 309-3, 309-4, 309-5. A third subset of the synthetic compounds, represented by beads 303-4, 303-9, 303-10, 303-N, bind to a second subset of the plurality biological counter targets, represented by second detectable labels 309-2, 309-6, 309-7, 309-8. As may be appreciated, the binding assay 330 is provided as a non-limiting example, and assays can include additional synthetic compounds, biological targets, biological counter targets, as well as different numbers within each sub-set, such as more or less, and/or respective synthetic compounds which do not bind to a copy of either of a biological target and a biological counter target.

Although FIG. 3A is described as a competitive binding assay, in some embodiments, the assay can be used as an associative binding assay. For example, the binding assay 330 can be screened to identify the second subset of synthetic compounds, represented by the beads 303-1, 303-5, 303-7, 303-8, that bind to the second subset of the plurality of biological targets, represented by first detectable labels 307-1, 307-4, 307-6, 307-7, and which bind to the first subset of the plurality biological counter targets, represented by second detectable labels 309-1, 309-3, 309-4, 309-5. As may be appreciated, “first”, “second, and “third” is not intended to implicate an order and, in such embodiments, the second subset of synthetic compounds can be what are screened for and can be referred to as a first subset of synthetic compounds which bind to a first subset of the biological targets and a first subset of the plurality of biological counter targets.

FIG. 3B illustrates different binding arrangements that can occur in response to the exposure of the beads 303 with an attached synthetic compound to the biological target 306 and the counter target 308, as shown by 334. A first binding arrangement 336 includes the synthetic compound on the bead 303 being bound to the biological target 306 in a manner that blocks interactions between the biological target 306 and the biological counter target 308. In the first binding arrangement 336, a signal associated with the first detectable label 307 can be detected. A second binding arrangement 338 includes the synthetic compound on the bead 303 being bound to the biological target 306 in a manner that allows for interactions between the biological target 306 and the biological counter target 308 to occur. In the second binding arrangement 338, a first signal associated with the first detectable label 307 and a second signal associated with the second detectable label 309 can be detected. A third binding arrangement 340 includes the synthetic compound on the bead 303 being bound to the counter biological target 308 in a manner that blocks interactions between the biological target 306 and the biological counter target 308. In the third binding arrangement 340, a signal associated with the second detectable label 309 can be detected. Although not illustrated, at least some of the synthetic compounds can bind to neither of the biological target 306 and the biological counter target 308.

As previously described, embodiments are not limited to binding assays involving a biological target and biological counter target. In some embodiments, multiple biological targets can be screened. For example, a plurality of synthetic compounds can be screened to identify synthetic compound which directly bind two (or more) biological targets, or that selectively bind (e.g., bind one and not the other or more) to respective ones of the two or more biological targets.

FIG. 3C illustrates different binding arrangements that can occur in response to the exposure of the bead 303 with an attached synthetic compound to a first biological target 323 labeled with the first detected label 307 and a second biological target 325 labeled with the second detected label 309, as shown by 333. A first binding arrangement 335 includes the synthetic compound on the bead 303 being bound to the first biological target 323 and the second biological counter target 325. In the first binding arrangement 335, a first signal associated with the first detectable label 307 and a second signal associated with the second detectable label 309 can be detected. A second binding arrangement 337 includes the synthetic compound on the bead 303 being bound to the first biological target 323 and not the second biological target 325. In the second binding arrangement 337, a signal associated with the first detectable label 307 can be detected. A third binding arrangement 339 includes the synthetic compound on the bead 303 being bound to the second biological target 325 and not the first biological target 323. In the third binding arrangement 339, a signal associated with the second detectable label 309 can be detected. Although not illustrated, at least some of the synthetic compounds can bind to neither of the first biological target 323 and the second biological target 325. In some embodiments, a particular synthetic compound can bind to multiple copies of the same biological target, such as two or more of the first biological target 323 and/or two or more of the second biological target 325, which may increase the intensity and/or number of signals detected as compared to a synthetic compound bound to one copy of the first biological target 323 and/or the second biological target 325. The signal intensities and/or number of signals may be compared to identify such binding.

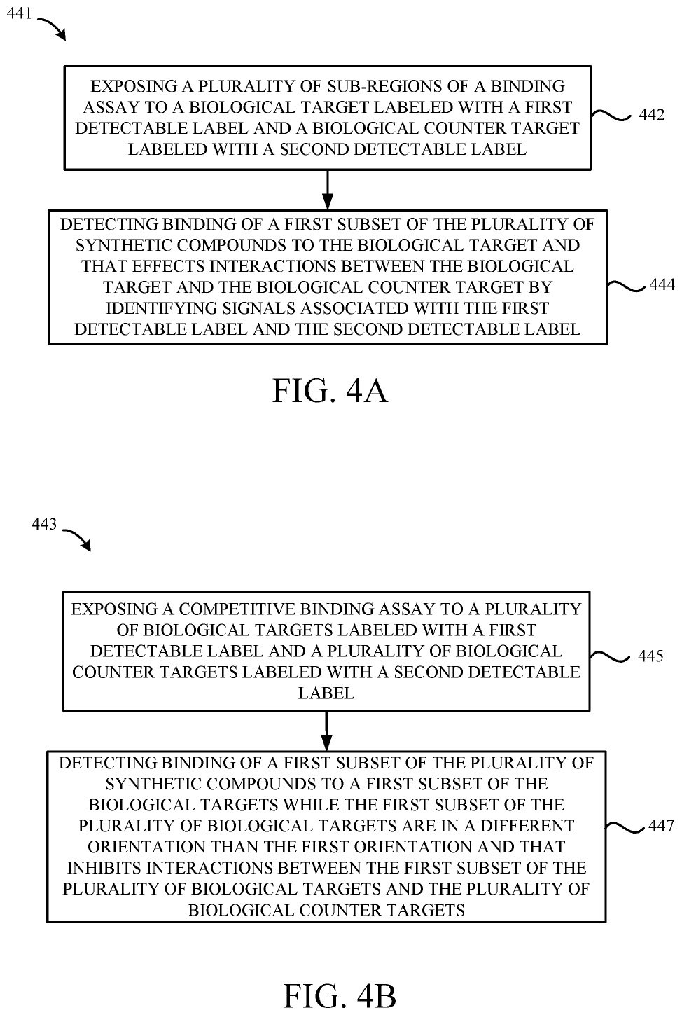

FIGS. 4A-4B illustrate example methods of screening a plurality of synthetic compounds for binding to a biological target that effects binding to a biological counter target using a binding assay, in accordance with the present disclosure.

FIG. 4A illustrates an example method of screening a plurality of synthetic compounds for binding to a biological target using a binding assay. The method 441 can be implemented by or using any of the above described binding assays, such as the binding assay 100 illustrated by FIG. 1 and/or the binding assay 330 illustrated by FIG. 3A.

At 442, the method 441 includes exposing a plurality of sub-regions of a binding assay to a biological target labeled with a first detectable label and a biological counter target labeled with a second detectable label. As previously described, each of the plurality of synthetic compounds are disposed on a bead and are distinguishable by mass spectrometry. Further, the biological counter target is configured to bind to the biological target when the biological target is in a first orientation. In some embodiments, exposing the assay to the biological target and the biological counter target (e.g., a plurality of copies of each) includes incubating the plurality of synthetic compounds with the biological target and the biological counter target, and optionally, after incubating, washing unbound biological targets and biological counter targets from the assay.

At 444, the method 441 includes detecting binding of a first subset of the plurality of synthetic compounds to the biological target and that effects interactions between the biological target and the biological counter target by identifying signals associated with the first detectable label and the second detectable label. For example, the binding assay is exposed to a plurality of biological targets and a plurality of the biological counter targets, and binding is detected between first subset of the plurality of synthetic compounds and a first subset of the plurality of biological targets while the first subset of the plurality of biological targets are in a different orientation than the first orientation. As another example, the binding is detected between a first subset of the plurality of synthetic compounds and a first subset of the plurality of biological targets while the first subset of the plurality of biological targets are in the first orientation and the first subset of the plurality of biological targets are bound to (a first subset of) the plurality of biological counter targets.

In some embodiments, detecting the binding of the first subset of the plurality of synthetic compounds includes identifying blocking of binding of the biological counter target (e.g., between respective ones of the plurality of biological counter targets) and the biological target (e.g., the first subset of the plurality of the biological targets) by identifying the signals associated with the second detectable label are below a threshold. For example, in some embodiments, the threshold can be zero, and in other embodiments, the threshold can be greater than zero to account for noise, such as 0.1 relative fluorescent units (RFU). Alternatively and/or in addition, in some embodiments, the first subset of the plurality of synthetic compounds can be identified by identifying signals associated with the first detectable label that are above a second threshold, which can be different than the threshold for the second detectable label.

In some embodiments, the method 441 further including isolating the first subset of the plurality of synthetic compounds and performing mass spectrometry to identify sequences of the first subset of the plurality of synthetic compounds. As previously described, the plurality of synthetic compounds include different subsets of a plurality of molecules, each of the plurality of molecules including a plurality of subgroups, such as various amino acids and other monomers or functional groups, and exhibiting a mass spectrometry characteristic that is distinguishable from mass spectrometry characteristics of other molecules of the plurality, and cleavable groups linking at least some of the different subsets of the plurality of molecules and/or one of the plurality of molecules and the bead. The method 441 can include separating the first subset of the plurality of synthetic compounds from the beads and the respective different subset of molecules forming the synthetic compounds of the first subset from one another, and sequencing the first subset of the plurality of synthetic compounds by identifying the mass spectrometry characteristics of the respective different subset of molecules via mass spectrometry

FIG. 4B illustrates an example method of screening a plurality of synthetic compounds for binding to a biological target using a competitive binding assay. The method 443 can include an implementation of the method 441 of FIG. 4A. At 445, the method 443 includes exposing a competitive binding assay to a plurality of biological targets labeled with a first detectable label and a plurality of biological counter targets labeled with a second detectable label. The competitive binding assay can first be exposed to the plurality of biological targets, followed by (and after incubating) the exposure to the plurality of biological counter targets. At 447, the method 443 includes detecting binding of a first subset of the plurality of synthetic compounds to a first subset of the plurality of biological targets such that the first subset of the plurality of biological targets are in a different orientation than the first orientation and that inhibits interactions between the first subset of the plurality of biological targets and the plurality of biological counter targets. In some embodiments, additional subsets of synthetic compounds can be identified, as previously described.

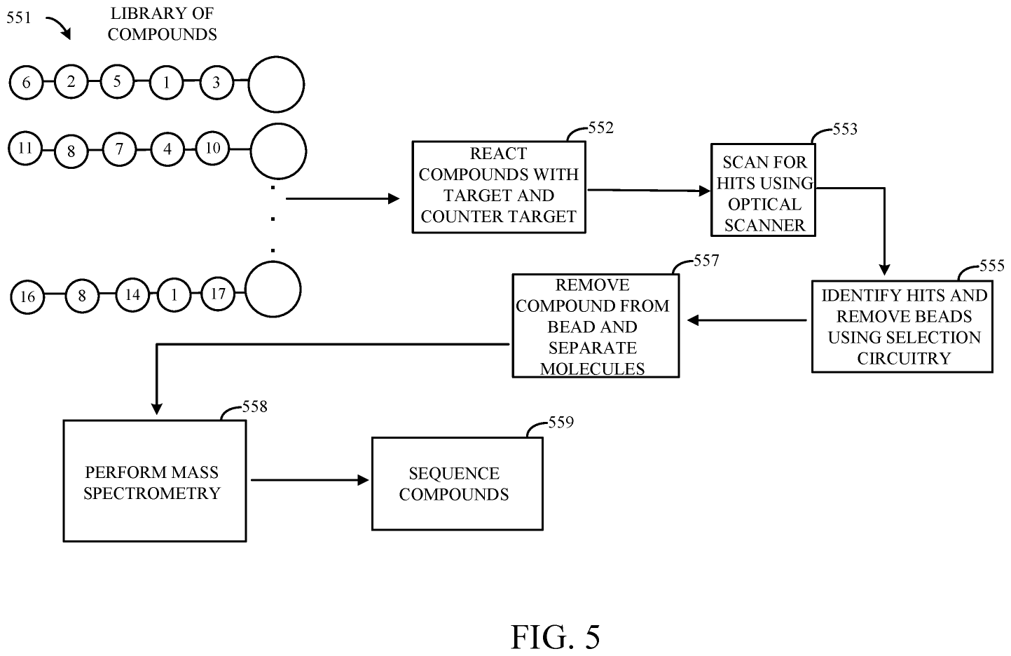

FIG. 5 illustrates an example process for screening a library of a plurality of synthetic compounds for binding to a biological target using a binding assay, in accordance with various embodiments. The library 551 can be formed according to at least some of substantially the same features and/or aspects as described by US Patent Application, Pub. No. US2021/0065848, as identified above.

The library of compounds, at 552, is reacted with a plurality of biological targets and biological counter targets, which are respectively labeled with different detectable labels, such as fluorescent labels. The synthetic compounds can be reacted on a screening plate, such as an assay, and non-reacted material is washed away. The screening plate is screened, at 553, for hits using an optical scanner. For example, the screening plate can be scanned to identify synthetic compounds that bind to a respective one of the labeled biological targets and prevent or mitigate binding between the biological target and a respective one of the labeled biological counter targets. At 555, hits are identified and the synthetic compounds are removed and plated using selection circuitry. Example selection circuitry includes a glass capillary (e.g., for manual picking) and/or a capillary extractor for automated picking such as the Automated Lab Solutions (ALS) CellCelector or flow sorting using a fluorescence-activated cell sorting (FACs) instrument. The synthetic compounds that are isolated are cleaved, at 557, to remove the synthetic compound from the bead and/or to separate the molecules from one another. The cleavage can be performed via chemical, physical (e.g., temperate), light induced and/or biological/enzymatic methods. The cleavage results in a mixture of the molecules, separated from one another, in solution. The mixture can be used to identify the sequence of the synthetic compound (e.g., what order the molecules are in the compound) by performing mass spectrometry, at 558, to identify the mass spectrometry characteristics of the molecules that form the synthetic compound. At 559, the mass spectrometry characteristics are used to sequence the synthetic compound including identifying the molecules in the synthetic compound, the position of each molecule in the sequence of the synthetic compound, and the sequence order of subgroups forming each molecule. For example, the mass spectrometry characteristics map to identification of the molecule, the respective position in the sequence of the synthetic compound, and the respective sequence of subgroups.

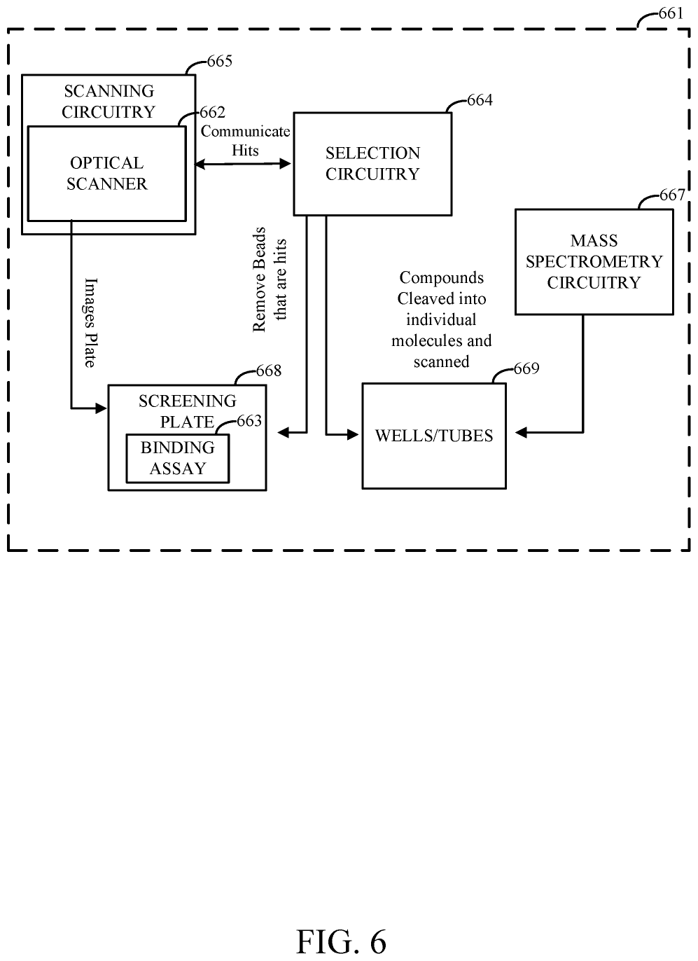

FIG. 6 illustrates an example apparatus used to screen a library of a plurality of synthetic compounds, in accordance with the present disclosure. As illustrated, the apparatus 661 includes a binding assay 663 and scanning circuitry 665. As previously described, the binding assay 663 includes a substrate, such as a screening plate 668, a plurality of synthetic compounds on beads that are distinguishable by mass spectrometry, a biological target labeled with a first detectable label, and a biological counter target labeled with a second detectable label, with the biological counter target configured to bind to the biological target when the biological target is in a first orientation.

The scanning circuitry 665 can identify a first subset of the first subset of the plurality of synthetic compounds that bind to the biological target that effects interactions between the biological target and the biological counter target. In some embodiments, the effects on interactions between the biological target and the biological counter target can include inhibiting, mitigating, or reducing binding between the biological target and the biological counter target. In other embodiments, the effects on interactions between the biological target and the biological counter target can include allowing or permitting for binding between the biological target and the biological counter target.

As previously described, as each sub-region includes a biological target and biological counter target, the binding assay includes a plurality of each. In some embodiments, the scanning circuitry 665 is to identify a first subset of the plurality of synthetic compounds that bind to a first subset of the biological targets such that the first subset of the plurality of biological targets are in a different orientation than the first orientation and that inhibit interactions between the first subset of the biological targets and the plurality of biological counter targets. For example, the scanning circuitry 665 can identify the first subset of the plurality of synthetic compounds based on a first fluorescent signal associated with the first detectable label, and a second fluorescent signal associated with the second detectable label.

In some embodiments, the scanning circuitry 665 is to identify a first subset of the plurality of synthetic compounds that bind to the first subset of the biological targets such that the first subset of the plurality of biological targets are in the first orientation and that allow or permit for interactions between the first subset of the biological targets and the plurality of biological counter targets. For example, the scanning circuitry 665 can identify the first subset of the plurality of synthetic compounds based on a first fluorescent signal associated with the first detectable label, and a second fluorescent signal associated with the second detectable label.

In some embodiments, the scanning circuitry 665 can provide a qualitative measure of binding affinity based a detected level (e.g., intensity) of a fluorescent signal associated with the first fluorescent label. In some embodiments, scanning circuitry is to provide a ratio of the biological target binding to the biological counter target binding based on detection of a first detected level of a fluorescent signal associated with the first detectable label and a second detected level of a fluorescent signal associated with the second detectable label.

In some embodiments, the apparatus 661 further includes selection circuitry 664 to isolate the first subset of the plurality of synthetic compounds. In some embodiments, the apparatus 661 includes mass spectrometry circuitry 667 to sequence the first subset of the plurality of synthetic compounds by identifying the mass spectrometry characteristics of the respective subsets of molecules of the first subset of the plurality of synthetic compounds. For example, the apparatus 661 and/or scanning circuitry 665 can include an optical scanner 662, a screening plate 668, selection circuitry 664, wells and/or tubes 669, and mass spectrometry circuitry 667. The above-described methods and/or polymer beads can be used to generate a library of 10\hat{ }8 to 10\hat{ }9 (or more) compounds. The library can be screened for hits to a biological target and which do or do not include the biological counter target using an optical scanner 662. An example of an optical scanner 662 is a fiber optic scanner, which includes a fiber optic bundle array, a laser, and imaging circuitry (e.g., camera), such as FAST, as previously described.

In some embodiments, a competitive binding assay can be performed with the beads to identify synthetic compounds which bind to the biological target and which prevent or mitigate binding between the biological target and a biological counter target. The detected activity is assessed via a fluorescent readout of the assay using the optical scanner 662. Identified beads that are suspected of exhibiting the competitive binding activity are identified based on the scan and removed from the screening plate 668 and placed in wells and/or tubes 669 using selection circuitry 664 (e.g., a robot). The removed beads are further processed to cleave the compounds from the beads and, optionally, into individual molecules. The molecules in solution are then read out using mass spectrometry circuitry 667 to identify the molecules, the respective position of each molecule in the sequence of the compound, and the respective sequence of subgroups (e.g., amino acids and/or other monomers or functional groups) forming each molecule.

Circuitry, such as logic circuitry, can be in communication with the mass spectrometry circuitry 667 and can receive the results from the mass spectrometry process. The circuitry can receive or otherwise have the map that identifies the possible molecules at each position in compounds of the library and the respective mass spectrometry characteristics. The map, and/or other data, can also identify the sequence of subgroups forming each of the possible molecules. In response to receiving the results from the mass spectrometry circuitry 667, the circuitry compares the mass spectrometry characteristics in the map to the received results to identify molecules in the synthetic compound, the sequence order of the molecules, and the sequence order of subgroups forming the synthetic compound.

Various embodiments are directed to specific synthetic compounds identified using the above described apparatuses and/or assays, which include competitive binding assays and other types of binding assays. The synthetic compounds comprise N plurality of molecules, each of the N plurality of molecules being formed of M amino acids, wherein N is between six and nine and M is between three and five; and cleavable groups linking at least some of the N plurality of molecules. The synthetic compound can be configured to bind to a biological target selected from a group consisting of: K-Ras, IL-6. TNFa, IL-6R, and ASGPR.

Libraries of synthetic compounds are not limited the synthetic compounds comprising N plurality of molecules, each of the N plurality of molecules being formed of M amino acids, wherein N is between six and nine and M is between three and five. In some embodiments, the synthetic compounds of a library can include N of between two and fifty, and M of between two and ten.

In some embodiments, the monomers each include an L-amino acid or a D-amino acid, and the cleavable groups include PAM.

In some embodiments, N is six and M is five, and the biological target includes K-Ras or ASGPR. In some embodiments, the synthetic compound has a binding affinity for K-Ras of between 18 and 180 nM. In some embodiments, the synthetic compound has a binding affinity for ASGPR of between 0.22 and 330 nM.

In some embodiments, the synthetic compound binds to the biological target of K-Ras and inhibits PPI between K-Ras and a biological counter target of Raf.

In some embodiments, N is nine and M is three, and the biological target includes IL-6, TNFa, or IL-6R. In some embodiments, the synthetic compound has a binding affinity for IL-6 of between 25 and 500 nM. In some embodiments, the synthetic compound has a binding affinity for IL-6R of between 0.6 and 330 nM. In some embodiments, the synthetic compound has a binding affinity for TNFα of between 0.3 and 270 nM.

In some embodiments, the synthetic compound binds to the biological target of IL-6 and inhibits PPI between IL-6 and a biological counter target of IL-6R. In other embodiments, the synthetic compound binds to the biological target of IL-6R and inhibits PPI between IL-6R and a biological counter target of IL-6.

In some embodiments, the synthetic compound is at least 70% similar to a compound selected from Table 13. In some embodiments, the synthetic compound is at least 75%, at least 80%, at least 85%, at least 95%, at least 96%, at least 97%, at least 98%, at least 99%, or 100% similar to a compound selected from Table 13. In some examples, the synthetic compound is at least 75%, at least 80%, at least 85%, at least 95%, at least 96%, at least 97%, at least 98%, at least 99%, or 100% similar to a compound selected from Table 13 with the fluorescein removed.

In some embodiments, the synthetic compound is at least 70%, at least 75%, at least 80%, at least 85%, at least 90% at least 95%, 96%, 97%, 98%, 99%, or 100% similar to a compound selected from:
(D)Trp-(D)Pro-(D)His-(D)Tyr-(L)Val-PAM-Gly-(D)Val-(D)Pro-(D)Arg-(L)Ala-PAM-(D)Pro-(D)Ser-(D)Glu-(D)Arg-Gly-PAM-(D)Pro-(D)Lys-(D)Tyr-(D)Arg-(L)Phe-PAM-(D)Ser-(D)Ser-Gly-(D)Asn-(L)Phe-PAM-Gly-(D)Trp-(D)His-(D)Arg-(L)Ala-PAM;
(D)Trp-(D)Pro-(D)His-(D)Tyr-(L)Val-PAM-Gly-(D)Val-(D)Pro-(D)Arg-(L)Ala-PAM-(D)Pro-(D)Ser-(D)Glu-(D)Arg-Gly-PAM-(D)Pro-(D)Lys-(D)Tyr-(D)Arg-(L)Phe-PAM-(D)Pro-Gly-(D)Arg-(D)Trp-Gly-PAM-(D)Tyr-(D)Pro-(D)Arg-(D)Glu-Gly-PAM;
(D)Leu-(D)Pro-(D)Glu-(D)Ser-(L)Phe-PAM-Gly-(D)Val-(D)Pro-(D)Arg-(L)Ala-PAM-(D)Pro-(D)Ser-(D)Glu-(D)Arg-Gly-PAM-(D)Pro-(D)Lys-(D)Tyr-(D)Arg-(L)Phe-PAM-(D)Arg-(D)Tyr-(D)Thr-(D)Glu-(L)Ala-PAM-Gly-(D)Trp-(D)His-(D)Arg-(L)Ala-PAM;
(D)Leu-(D)Pro-(D)Glu-(D)Ser-(L)Phe-PAM-Gly-(D)Val-(D)Pro-(D)Arg-(L)Ala-PAM-(D)Pro-(D)Ser-(D)Glu-(D)Arg-Gly-PAM-(D)Pro-(D)Lys-(D)Tyr-(D)Arg-(L)Phe-PAM-(D)Arg-(D)Tyr-(D)Thr-(D)Glu-(L)Ala-PAM-(D)Tyr-(D)Pro-(D)Arg-(D)Glu-Gly-PAM;
(D)Trp-(D)Pro-(D)His-(D)Tyr-(L)Val-PAM-(D)Pro-(D)Ser-Gly-(D)Ser-(L)Ala-PAM-(D)Pro-(D)Ser-(D)Glu-(D)Arg-Gly-PAM-(D)Pro-(D)Lys-(D)Tyr-(D)Arg-(L)Phe-PAM-(D)Pro-Gly-(D)Arg-(D)Trp-Gly-PAM-(D)Tyr-(D)Pro-(D)Arg-(D)Glu-Gly-PAM;
(D)Ser-(D)Pro-(D)His-(D)Glu-(L)Phe-PAM-(D)Pro-(D)Ser-Gly-(D)Ser-(L)Ala-PAM-(D)Thr-(D)Ser-(D)Arg-(D)Glu-(L)Leu-PAM-(D)Pro-(D)Lys-(D)Tyr-(D)Arg-(L)Phe-PAM-(D)Arg-(D)Tyr-(D)Thr-(D)Glu-(L)Ala-PAM-Gly-(D)Trp-(D)His-(D)Arg-(L)Ala- PAM;
(D)Trp-(D)Pro-(D)His-(D)Tyr-(L)Val-PAM-Gly-(D)Val-(D)Pro-(D)Arg-(L)Ala-PAM-Gly-(D)Pro-(D)Arg-(D)Ser-(L)Phe-PAM-(D)Tyr-(D)Thr-(D)Pro-(D)Arg-(L)Ala-PAM-(D)Pro-Gly-(D)Arg-(D)Trp-Gly-PAM-(D)Ser-(D)Pro-(D)Tyr-(D)Leu-(L)Ala-PAM;
(D)Ser-(D)Phe-(D)Pro-(D)Glu-(L)Leu-PAM-Gly-(D)Val-(D)Pro-(D)Arg-(L)Ala-PAM-(D)Pro-(D)Ser-(D)Glu-(D)Arg-Gly-PAM-(D)Pro-(D)Lys-(D)Tyr-(D)Arg-(L)Phe-PAM-(D)Arg-(D)Phe-(D)Pro-(D)Val-Gly-PAM-Gly-(D)Trp-(D)His-(D)Arg-(L)Ala-PAM;
(D)Glu-(D)Pro-(D)Thr-(D)Glu-(L)Leu-PAM-Gly-(D)Val-(D)Pro-(D)Arg-(L)Ala-PAM-(D)Pro-(D)Ser-(D)Glu-(D)Arg-Gly-PAM-(D)Pro-(D)Lys-(D)Tyr-(D)Arg-(L)Phe-PAM-(D)Arg-(D)Tyr-(D)Thr-(D)Glu-(L)Ala-PAM-Gly-(D)Trp-(D)His-(D)Arg-(L)Ala-PAM;
(D)Tyr-(D)His-(D)Pro-(D)Trp-(L)Val-PAM-Gly-(D)Lys-(D)His-(D)Asn-(L)Phe-PAM-(D)Pro-(D)Ser-(D)Glu-(D)Arg-Gly-PAM-(D)Pro-(D)Lys-(D)Tyr-(D)Arg-(L)Phe-PAM-(D)Pro-Gly-(D)Arg-(D)Trp-Gly-PAM-(D)Tyr-(D)Pro-(D)Arg-(D)Glu-Gly-PAM;
(D)Leu-(D)Pro-(D)Glu-(D)Ser-(L)Phe-PAM-(D)Phe-(D)Glu-(D)Lys-(D)Arg-(L)Leu-PAM-(D)Pro-(D)Ser-(D)Glu-(D)Arg-Gly-PAM-(D)Pro-(D)Lys-(D)Tyr-(D)Arg-(L)Phe-PAM-(D)Pro-Gly-(D)Arg-(D)Trp-Gly-PAM-Gly-(D)Trp-(D)His-(D)Arg-(L)Ala-PAM;
(D)Leu-(D)Pro-(D)Glu-(D)Ser-(L)Phe-PAM-Gly-(D)Val-(D)Pro-(D)Arg-(L)Ala-PAM-(D)Lys-(D)Thr-(D)Glu-(D)Ala-(L)Leu-PAM-(D)Tyr-(D)Thr-(D)Pro-(D)Arg-(L)Ala-PAM-(D)Pro-Gly-(D)Arg-(D)Trp-Gly-PAM-Gly-(D)Trp-(D)His-(D)Arg-(L)Ala-PAM;
(D)Ser-(D)Pro-(D)His-(D)Glu-(L)Phe-PAM-Gly-(D)Val-(D)Pro-(D)Arg-(L)Ala-PAM-(D)Pro-(D)Ser-(D)Glu-(D)Arg-Gly-PAM-(D)Pro-(D)Lys-(D)Tyr-(D)Arg-(L)Phe-PAM-(D)Ala-(D)Glu-(D)Leu-(D)Lys-(L)Ala-PAM-Gly-(D)Trp-(D)His-(D)Arg-(L)Ala-PAM;
(D)Trp-(D)Pro-(D)His-(D)Tyr-(L)Val-PAM-(D)Phe-(D)Glu-(D)Lys-(D)Arg-(L)Leu-PAM-(D)Pro-(D)Ser-(D)Glu-(D)Arg-Gly-PAM-(D)Pro-(D)Lys-(D)Tyr-(D)Arg-(L)Phe-PAM-(D)Pro-Gly-(D)Arg-(D)Trp-Gly-PAM-(D)His-(D)Tyr-(D)Ser-(D)Glu-(L)Phe-PAM; fluorescein-(D)Leu-(D)Pro-(D)Glu-(D)Ser-(L)Phe-phenyl-acetamido-methylene (PAM)-(D)Phe-(D)Glu-(D)Lys-(D)Arg-(L)Leu-PAM-Gly-(D)Pro-(D)Arg-(D)Ser-(L)Phe-PAM-(D)Pro-(D)Lys-(D)Tyr-(D)Arg-(L)Phe-PAM-(D)Arg-(D)Phe-(D)Pro-(D)Val-Gly-PAM-(D)Leu-(D)Pro-(D)Arg-(D)Thr-Gly-PAM;
(D)Leu-(D)-Pro-(D)Glu-(D)Ser-(L)Phe-PAM-(D)Phe-(D)Glu-(D)Lys-(D)Arg-(L)Leu-PAM-Gly-(D)Pro-(D)Arg-(D)Ser-(L)Phe-PAM-(D)Pro-(D)Arg-(D)His-Gly-(L)Leu-PAM-(D)Arg-(D)Phe-(D)Pro-(D)Val-Gly-PAM-(D)Leu-(D)Pro-(D)Arg-(D)Thr-Gly-PAM;
(D)Leu-(D)-Pro-(D)Glu-(D)Ser-(L)Phe-PAM-(D)Phe-(D)Glu-(D)Lys-(D)Arg-(L)Leu-PAM-Gly-(D)Pro-(D)Arg-(D)Ser-(L)Phe-PAM-(D)Tyr-(D)Thr-(D)Pro-(D)Arg-(L)Ala-PAM-(D)Arg-(D)Phe-(D)Pro-(D)Val-Gly-PAM-(D)Leu-(D)Pro-(D)Arg-(D)Thr-Gly- PAM;
(D)Leu-(D)Pro-(D)Glu-(D)Ser-(L)Phe-PAM-(D)Phe-(D)Glu-(D)Lys-(D)Arg-(L)Leu-PAM-(D)His-(D)Ser-(D)Pro-(D)Arg-(L)Val-PAM-(D)Pro-(D)Lys-(D)Tyr-(D)Arg-(L)Phe-PAM-(D)Arg-(D)Phe-(D)Pro-(D)Val-Gly-PAM-(D)Leu-(D)Pro-(D)Arg-(D)Thr-Gly- PAM;
(D)Leu-(D)Pro-(D)Glu-(D)Ser-(L)Phe-PAM-(D)Phe-(D)Glu-(D)Lys-(D)Arg-(L)Leu-PAM-(D)Tyr-(D)Asn-(D)Arg-(D)His-(L)Phe-PAM-(D)Asn-(D)Pro-(D)Leu-(D)His-(L)Leu-PAM-(D)Arg-(D)Phe-(D)Pro-(D)Val-Gly-PAM-(D)Leu-(D)Pro-(D)Arg-(D)Thr-Gly- PAM;
(D)Leu-(D)Pro-(D)Glu-(D)Ser-(L)Phe-PAM-(D)Phe-(D)Glu-(D)Lys-(D)Arg-(L)Leu-PAM-(D)Tyr-(D)Asn-(D)Arg-(D)His-(L)Phe-PAM-(D)Arg-(D)Ser-(D)Glu-(D)Trp-(L)Leu-PAM-(D)Arg-(D)Phe-(D)Pro-(D)Val-Gly-PAM-(D)Leu-(D)Pro-(D)Arg-(D)Thr-Gly- PAM;
(D)Leu-(D)Pro-(D)Glu-(D)Ser-(L)Phe-PAM-(D)Phe-(D)Glu-(D)Lys-(D)Arg-(L)Leu-PAM-(D)Tyr-(D)Asn-(D)Arg-(D)His-(L)Phe-PAM-(D)Tyr-(D)Thr-(D)Pro-(D)Arg-(L)Ala-PAM-(D)Arg-(D)Phe-(D)Pro-(D)Val-Gly-PAM-(D)Leu-(D)Pro-(D)Arg-(D)Thr-Gly- PAM;
(D)Leu-(D)Pro-(D)Glu-(D)Ser-(L)Phe-PAM-Gly-(D)Val-(D)Pro-(D)Arg-(L)Ala-PAM-Gly-(D)Pro-(D)Arg-(D)Ser-(L)Phe-PAM-(D)Pro-(D)Arg-(D)His-Gly-(L)Leu-PAM-(D)Arg-(D)Phe-(D)Pro-(D)Val-Gly-PAM-(D)Leu-(D)Pro-(D)Arg-(D)Thr-Gly-PAM;
(D)Tyr-(D)Leu-(L)Leu-phenyl-acetamido-methylene (PAM)-(D)Tyr-(D)Ser-(L)Val-PAM-(D)Arg-(D)Ser-(L)Phe-PAM-(D)Trp-(D)Arg-(L)Leu-PAM-(D)Trp-(D)Arg-(L)Val-PAM-(D)Arg-(D)Tyr-(L)Leu-PAM-(D)Glu-(D)Tyr-(L)Val-PAM-(D)Lys-(D)Phe-(L)Phe- PAM-(D)Tyr-(D)Arg-(L)Phe-PAM;
(D)His-(D)Leu-(L)Phe-PAM-(D)Ala-(D)Ala-(L)Phe-PAM-(D)Pro-(D)Lys-(L)Ala-PAM-(D)Phe-(D)Arg-(L)Ala-PAM-(D)Ala-(D)Lys-(L)Phe-PAM-(D)Phe-(D)Thr-(L)Ala-PAM-(D)Lys-(D)Asn-(L)Leu-PAM-(D)Thr-(D)His-(L)Leu-PAM-(D)Phe-(D)Lys-Gly-PAM;
(D)Arg-(D)Trp-(L)Phe-PAM-(D)Glu-(D)Ser-(L)Phe-PAM-(D)Arg-(D)Ser-(L)Phe-PAM-(D)Trp-(D)Arg-(L)Leu-PAM-Gly-(D)Ala-(L)Val-PAM-(D)Asn-(D)Arg-(L)Phe-PAM-(D)Ala-(D)Ser-(L)Phe-PAM-(D)Pro-(D)Arg-(L)Ala-PAM-(D)Phe-(D)Lys-Gly-PAM;
(D)Arg-(D)Trp-(L)Phe-PAM-(D)Leu-(D)Arg-(L)Leu-PAM-(D)His-(D)Tyr-(L)Phe-PAM-(D)Trp-(D)Arg-(L)Leu-PAM-(D)His-(D)Lys-(L)Leu-PAM-(D)Ala-(D)Tyr-Gly-PAM-(D)His-(D)Glu-(L)Leu-PAM-(D)Pro-(D)Arg-(L)Ala-PAM-(D)Phe-(D)Lys-Gly-PAM;
(D)Ser-(D)Phe-(L)Phe-PAM-Gly-(D)Arg-(L)Leu-PAM-(D)Leu-(D)Lys-(L)Leu-PAM-(D)Trp-(D)Arg-(L)Leu-PAM-(D)Ala-(D)Lys-(L)Phe-PAM-(D)His-(D)Lys-(L)Phe-PAM-(D)Ala-(D)Arg-(L)Leu-PAM-(D)Thr-(D)His-(L)Leu-PAM-(D)Phe-(D)Arg-Gly-PAM;
(D)Tyr-(D)Leu-(L)Leu-PAM-Gly-(D)Arg-(L)Leu-PAM-(D)Arg-(D)Ser-(L)Phe-PAM-(D)Phe-(D)Arg-(L)Ala-PAM-(D)Glu-(D)Lys-(L)Phe-PAM-(D)Glu-(D)Arg-(L)Leu-PAM-(D)Ala-(D)Arg-(L)Leu-PAM-(D)Pro-(D)Arg-(L)Ala-PAM-(D)Tyr-(D)Arg-(L)Phe-PAM;
(D)Tyr-(D)Leu-(L)Leu-PAM-Gly-(D)Arg-(L)Leu-PAM-(D)Arg-(D)Ser-(L)Phe-PAM-(D)Pro-(D)Arg-(L)Val-PAM-(D)Trp-(D)Arg-(L)Val-PAM-(D)Asn-(D)Arg-(L)Phe-PAM-(D)Glu-(D)Tyr-(L)Val-PAM-(D)Pro-(D)Arg-(L)Ala-PAM-(D)Tyr-(D)Arg-(L)Phe-PAM;
(D)Thr-(D)Leu-(L)Phe-PAM-Gly-(D)Arg-(L)Leu-PAM-(D)Arg-(D)Ser-(L)Phe-PAM-(D)Pro-(D)Arg-(L)Val-PAM-(D)Trp-(D)Arg-(L)Val-PAM-(D)Asn-(D)Arg-(L)Phe-PAM-(D)Glu-(D)Tyr-(L)Val-PAM-(D)Pro-(D)Arg-Ala-PAM-Tyr-Arg-Phe-PAM;
(D)Arg-(D)Trp-(L)Phe-phenyl-acetamido-methylene (PAM)-(D)Pro-(D)Asn-(L)Val-PAM-(D)Asn-(D)Thr-(L)Leu-PAM-(D)Lys-(D)Glu-(L)Leu-PAM-(D)Glu-(D)Lys-(L)Phe-PAM-(D)Glu-(D)Trp-(L)Ala-PAM-(D)Ala-(D)Arg-(L)Leu-PAM-(D)Glu-(D)Arg-(L)Val- PAM-(D)Phe-(D)Lys-Gly-PAM;
(D)Tyr-(D)Leu-(L)Leu-PAM-(D)Glu-(D)Ser-(L)Phe-PAM-(D)Asn-(D)Thr-(L)Leu-PAM-(D)Lys-(D)Glu-(L)Leu-PAM-(D)Glu-(D)Lys-(L)Phe-PAM-(D)Glu-(D)Arg-(L)Leu-PAM-(D)Ala-(D)Arg-(L)Leu-PAM-(D)Trp-(D)Lys-(L)Leu-PAM-(D)Phe-(D)Lys-Gly-PAM;
(D)Arg-(D)Trp-(L)Phe-PAM-(D)Glu-(D)Ser-(L)Phe-PAM-(D)Asn-(D)Thr-(L)Leu-PAM-(D)Tyr-(D)Asn-(L)Phe-PAM-(D)Tyr-(D)Lys-(L)Ala-PAM-(D)Glu-(D)Arg-(L)Leu-PAM-(D)Ala-(D)Arg-(L)Leu-PAM-(D)Glu-(D)Arg-(L)Val-PAM-(D)Phe-(D)Lys-Gly-PAM;
(D)Asn-(D)Val-(L)Phe-PAM-(D)Glu-(D)Ser-(L)Phe-PAM-(D)Arg-(D)Ser-(L)Phe-PAM-(D)Lys-(D)Glu-(L)Leu-PAM-(D)Trp-(D)Arg-(L)Val-PAM-(D)Pro-(D)Glu-(L)Phe-PAM-(D)Ala-(D)Arg-(L)Leu-PAM-(D)Pro-(D)Arg-(L)Ala-PAM-(D)Phe-(D)Arg-Gly-PAM;
(D)Arg-(D)Trp-(L)Phe-PAM-(D)Leu-(D)Arg-(L)Leu-PAM-(D)Asn-(D)Thr-(L)Leu-PAM-(D)Pro-(D)Arg-(L)Val-PAM-Gly-(D)Ala-(L)Val-PAM-(D)Glu-(D)Arg-(L)Leu-PAM-(D)Ala-(D)Arg-(L)Leu-PAM-(D)Pro-(D)Arg-(L)Ala-PAM-(D)Phe-(D)Arg-Gly-PAM;
(D)Arg-(D)Trp-(L)Phe-PAM-(D)Pro-(D)Asn-(L)Val-PAM-(D)Pro-(D)Lys-(L)Ala-PAM-(D)Trp-(D)Arg-(L)Leu-PAM-(D)Ala-(D)Lys-(L)Phe-PAM-(D)Pro-(D)Glu-(L)Phe-PAM-(D)Ala-(D)Arg-(L)Leu-PAM-(D)Trp-(D)Lys-(L)Leu-PAM-(D)Phe-(D)Lys-Gly-PAM;
(D)Asn-(D)Val-(L)Phe-PAM-(D)Tyr-(D)Ser-(L)Val-PAM-(D)Arg-(D)Ser-(L)Phe-PAM-(D)Phe-(D)Arg-(L)Ala-PAM-(D)Tyr-(D)Lys-(L)Ala-PAM-(D)Glu-(D)Arg-(L)Leu-PAM-(D)Ala-(D)Arg-(L)Leu-PAM-(D)Pro-(D)Arg-(L)Ala-PAM-(D)Phe-(D)Lys-Gly-PAM;
(D)Arg-(D)Trp-(L)Phe-PAM-(D)Leu-(D)Arg-(L)Leu-PAM-(D)Phe-(D)Glu-(L)Val-PAM-(D)Lys-(D)Glu-(L)Leu-PAM-(D)Ala-(D)Lys-(L)Phe-PAM-(D)Asn-(D)Arg-(L)Phe-PAM-(D)Ala-(D)Arg-(L)Leu-PAM-(D)Trp-(D)Lys-(L)Leu-PAM-(D)Ala-(D)Val-(L)Leu-PAM;
(D)Lys-(D)Val-(L)Leu-phenyl-acetamido-methylene (PAM)-(D)Ala-(D)Ala-(L)Phe-PAM-(D)Leu-(D)Lys-(L)Leu-PAM-(D)Phe-(D)Lys-(L)Leu-PAM-(D)Trp-(D)Arg-(L)Val-PAM-(D)Ser-(D)Ser-(L)Phe-PAM-(D)His-(D)Glu-(L)Leu-PAM-(D)Phe-(D)Lys-(L)Phe- PAM-(D)Thr-(D)Glu-(L)Leu-PAM;
(D)Thr-(D)Leu-(L)Phe-PAM-(D)Leu-(D)Arg-(L)Leu-PAM-(D)Leu-(D)Lys-(L)Leu-PAM-(D)Trp-(D)Arg-(L)Leu-PAM-(D)Ala-(D)Lys-(L)Phe-PAM-(D)Glu-(D)Ser-(L)Val-PAM-(D)Pro-(D)Val-Gly-PAM-(D)Glu-(D)Tyr-(L)Phe-PAM-(D)Tyr-(D)Arg-(L)Phe-PAM;
(D)Lys-(D)Val-(L)Leu-PAM-Gly-(D)Arg-(L)Leu-PAM-(D)Phe-(D)Glu-(L)Val-PAM-(D)Pro-(D)Arg-(L)Val-PAM-(D)Ala-(D)Lys-(L)Phe-PAM-(D)Glu-(D)Ser-(L)Val-PAM-(D)Pro-(D)Val-Gly-PAM-(D)Thr-(D)His-(L)Leu-PAM-(D)Tyr-(D)Arg-(L)Phe-PAM;
(D)Lys-(D)Val-(L)Leu-PAM-(D)Leu-(D)Arg-(L)Leu-PAM-(D)Phe-(D)Glu-(L)Val-PAM-(D)Phe-(D)Arg-(L)Ala-PAM-(D)Ala-(D)Lys-(L)Phe-PAM-(D)Phe-(D)Thr-(L)Ala-PAM-(D)Glu-(D)Tyr-(L)Val-PAM-Gly-(D)Glu-(L)Leu-PAM-(D)Trp-(D)Glu-(L)Val-PAM;
(D)Lys-(D)Val-(L)Leu-PAM-(D)Leu-(D)Arg-(L)Leu-PAM-(D)Phe-(D)Glu-(L)Val-PAM-(D)Trp-(D)Arg-(L)Leu-PAM-(D)Ala-(D)Lys-(L)Phe-PAM-(D)Arg-(D)Tyr-(L)Leu-PAM-(D)Pro-(D)Val-Gly-PAM-(D)Lys-(D)Phe-(L)Phe-PAM-(D)His-(D)Tyr-(L)Val-PAM;
(D)Tyr-(D)Leu-(L)Leu-PAM-(D)Leu-(D)Arg-(L)Leu-PAM-(D)Leu-(D)Lys-(L)Leu-PAM-(D)Phe-(D)Arg-(L)Ala-PAM-(D)Ala-(D)Lys-(L)Phe-PAM-(D)Asn-(D)Arg-(L)Phe-PAM-(D)Ala-(D)Arg-(L)Leu-PAM-(D)Trp-(D)Lys-(L)Leu-PAM-(D)Pro-(D)Ala-(L)Ala-PAM;
(D)Lys-(D)Val-(L)Leu-PAM-(D)Leu-(D)Arg-(L)Leu-PAM-(D)Arg-(D)Val-(L)Leu-PAM-(D)Phe-(D)Arg-(L)Ala-PAM-(D)Tyr-(D)Lys-(L)Ala-PAM-(D)Glu-(D)Arg-(L)Leu-PAM-(D)Pro-(D)Val-Gly-PAM-(D)Lys-(D)Phe-(L)Phe-PAM-(D)His-(D)Glu-(L)Val-PAM;
(D)Lys-(D)Val-(L)Leu-PAM-(D)Leu-(D)Arg-(L)Leu-PAM-(D)Phe-(D)Glu-(L)Val-PAM-(D)Phe-(D)Lys-(L)Leu-PAM-(D)Pro-(D)Glu-(L)Leu-PAM-(D)Phe-(D)Thr-(L)Ala-PAM-(D)His-(D)Glu-(L)Leu-PAM-(D)Glu-(D)Tyr-(L)Phe-PAM-(D)Thr-(D)Ser-(L)Val-PAM;
(D)Lys-(D)Val-(L)Leu-PAM-(D)Leu-(D)Arg-(L)Leu-PAM-(D)Arg-(D)Val-(L)Leu-PAM-(D)Phe-(D)Arg-(L)Ala-PAM-(D)Ala-(D)Lys-(L)Phe-PAM-(D)Asn-(D)Arg-(L)Phe-PAM-(D)Pro-(D)Val-Gly-PAM-(D)Glu-(D)Arg-(L)Val-PAM-(D)Tyr-(D)Arg-(L)Phe-PAM;
(D)Tyr-(D)Leu-(L)Leu-PAM-Gly-(D)Arg-(L)Leu-PAM-(D)Phe-(D)Glu-(L)Val-PAM-(D)Trp-(D)Arg-(L)Leu-PAM-(D)Trp-(D)Arg-(L)Val-PAM-(D)Arg-(D)Tyr-(L)Leu-PAM-(D)Pro-Gly-(L)Val-PAM-(D)His-(D)Ser-(L)Leu-PAM-(D)Phe-(D)Lys-Gly-PAM;
(D)Thr-(D)Leu-(L)Phe-PAM-(D)Leu-(D)Arg-(L)Leu-PAM-(D)Phe-(D)Glu-(L)Val-PAM-(D)Phe-(D)Arg-(L)Ala-PAM-(D)Ala-(D)Lys-(L)Phe-PAM-(D)Arg-(D)Tyr-(L)Leu-PAM-(D)Pro-Gly-(L)Val-PAM-(D)Trp-(D)Lys-(L)Leu-PAM-(D)Tyr-(D)Arg-(L)Phe-PAM;
(D)Thr-(D)Leu-(L)Phe-PAM-(D)Leu-(D)Arg-(L)Leu-PAM-(D)Phe-(D)Glu-(L)Val-PAM-(D)Phe-(D)Lys-(L)Leu-PAM-(D)Pro-(D)Glu-(L)Leu-PAM-(D)Pro-(D)Glu-(L)Phe-PAM-(D)His-(D)Glu-(L)Leu-PAM-(D)Pro-(D)Arg-(L)Ala-PAM-(D)His-(D)Tyr-(L)Val-PAM;
(D)Lys-(D)Val-(L)Leu-PAM-(D)Leu-(D)Arg-(L)Leu-PAM-(D)Phe-(D)Glu-(L)Val-PAM-(D)Phe-(D)Arg-(L)Ala-PAM-(D)Tyr-(D)Lys-(L)Ala-PAM-(D)Glu-(D)Ser-(L)Val-PAM-(D)Pro-(D)Val-Gly-PAM-(D)Pro-(D)Arg-(L)Ala-PAM-(D)Phe-(D)Lys-Gly-PAM;
(D)Tyr-(D)Leu-(L)Leu-PAM-(D)Leu-(D)Arg-(L)Leu-PAM-(D)Phe-(D)Glu-(L)Val-PAM-(D)Pro-(D)Arg-(L)Val-PAM-(D)Trp-(D)Arg-(L)Val-PAM-(D)Arg-(D)Tyr-(L)Leu-PAM-(D)Glu-(D)Val-(L)Val-PAM-(D)Thr-(D)His-(L)Leu-PAM-(D)Tyr-(D)Arg-(L)Phe-PAM;
(D)Lys-(D)Val-(L)Leu-PAM-(D)Leu-(D)Arg-(L)Leu-PAM-(D)Arg-(D)Val-(L)Leu-PAM-(D)Phe-(D)Lys-(L)Leu-PAM-(D)Ala-(D)Lys-(L)Phe-PAM-(D)Phe-(D)Thr-(L)Ala-PAM-(D)Pro-Gly-(L)Val-PAM-(D)Lys-(D)Phe-(L)Phe-PAM-(D)Ala-(D)Val-(L)Leu-PAM;
(D)Lys-(D)Val-(L)Leu-PAM-(D)Trp-(D)His-(L)Ala-PAM-(D)Phe-(D)Glu-(L)Val-PAM-(D)Trp-(D)Arg-(L)Leu-PAM-(D)Tyr-(D)Lys-(L)Ala-PAM-(D)Asn-(D)Arg-(L)Phe-PAM-(D)Pro-(D)Val-Gly-PAM-(D)Pro-(D)Arg-(L)Ala-PAM-(D)Ala-(D)Lys-Gly-PAM;
(D)Lys-(D)Val-(L)Leu-PAM-Gly-(D)Arg-(L)Leu-PAM-(D)Arg-(D)Val-(L)Leu-PAM-(D)Trp-(D)Arg-(L)Leu-PAM-(D)Trp-(D)Arg-(L)Val-PAM-(D)Ser-(D)Ser-(L)Phe-PAM-(D)Ala-(D)Ser-(L)Phe-PAM-(D)Trp-(D)Lys-(L)Leu-PAM-(D)Tyr-(D)Arg-(L)Phe-PAM;
(D)Lys-(D)Val-(L)Leu-PAM-(D)Ala-(D)Ala-(L)Phe-PAM-(D)Phe-(D)Glu-(L)Val-PAM-(D)Pro-(D)Arg-(L)Val-PAM-(D)Ala-Gly-(L)Phe-PAM-(D)Glu-(D)Arg-(L)Leu-PAM-(D)Pro-(D)Val-Gly-PAM-(D)Trp-(D)Lys-(L)Leu-PAM-(D)Pro-(D)Ala-(L)Ala-PAM;
(D)Lys-(D)Val-(L)Leu-PAM-(D)Leu-(D)Arg-(L)Leu-PAM-(D)Phe-(D)Glu-(L)Val-PAM-(D)Phe-(D)Arg-(L)Ala-PAM-(D)Ala-(D)Lys-(L)Phe-PAM-(D)Asn-(D)Arg-(L)Phe-PAM-(D)Pro-(D)Val-Gly-PAM-(D)Trp-(D)Lys-(L)Leu-PAM-(D)Tyr-(D)Arg-(L)Phe-PAM;
(D)Lys-(D)Val-(L)Leu-PAM-Gly-(D)Arg-(L)Leu-PAM-(D)Leu-(D)Lys-(L)Leu-PAM-(D)Pro-(D)Arg-(L)Val-PAM-(D)Ala-(D)Lys-(L)Phe-PAM-(D)Glu-(D)Trp-(L)Ala-PAM-(D)His-(D)Glu-(L)Leu-PAM-(D)Glu-(D)Arg-(L)Val-PAM-(D)Thr-(D)Ser-(L)Val-PAM;
(D)Ser-(D)Val-(L)Leu-PAM-Gly-(D)Arg-(L)Leu-PAM-(D)Phe-(D)Glu-(L)Val-PAM-(D)Phe-(D)Lys-(L)Leu-PAM-(D)Tyr-(D)Lys-(L)Ala-PAM-(D)Glu-(D)Trp-(L)Ala-PAM-(D)Ala-(D)Arg-(L)Leu-PAM-(D)Glu-(D)Tyr-(L)Phe-PAM-(D)Tyr-(D)Arg-(L)Phe-PAM;
(D)Tyr-(D)Leu-(L)Leu-PAM-(D)Leu-(D)Arg-(L)Leu-PAM-(D)Leu-(D)Lys-(L)Leu-PAM-(D)Phe-(D)Arg-(L)Ala-PAM-(D)Ala-(D)Lys-(L)Phe-PAM-(D)Phe-(D)Thr-(L)Ala-PAM-(D)Pro-(D)Val-Gly-PAM-(D)Thr-(D)His-(L)Leu-PAM-(D)Thr-(D)Ser-(L)Val-PAM; and
(D)Lys-(D)Val-(L)Leu-PAM-Gly-(D)Arg-(L)Leu-PAM-(D)Phe-(D)Glu-(L)Val-PAM-(D)Phe-(D)Arg-(L)Ala-PAM-(D)Trp-(D)Arg-(L)Val-PAM-(D)Glu-(D)Arg-(L)Leu-PAM-(D)His-(D)Glu-(L)Leu-PAM-(D)Lys-(D)Phe-(L)Phe-PAM-(D)Tyr-(D)Arg-(L)Phe-PAM.

In some embodiments, the biological target is K-Ras and the synthetic compound is at least 70% similar to a compound selected from:
fluorescein-(D)Trp-(D)Pro-(D)His-(D)Tyr-(L)Val-PAM-Gly-(D)Val-(D)Pro-(D)Arg-(L)Ala-PAM-(D)Pro-(D)Ser-(D)Glu-(D)Arg-Gly-PAM-(D)Pro-(D)Lys-(D)Tyr-(D)Arg-(L)Phe-PAM-(D)Ser-(D)Ser-Gly-(D)Asn-(L)Phe-PAM-Gly-(D)Trp-(D)His-(D)Arg- (L)Ala-PAM;
fluorescein-(D)Trp-(D)Pro-(D)His-(D)Tyr-(L)Val-PAM-Gly-(D)Val-(D)Pro-(D)Arg-(L)Ala-PAM-(D)Pro-(D)Ser-(D)Glu-(D)Arg-Gly-PAM-(D)Pro-(D)Lys-(D)Tyr-(D)Arg-(L)Phe-PAM-(D)Pro-Gly-(D)Arg-(D)Trp-Gly-PAM-(D)Tyr-(D)Pro-(D)Arg-(D)Glu- Gly-PAM;
fluorescein-(D)Leu-(D)Pro-(D)Glu-(D)Ser-(L)Phe-PAM-Gly-(D)Val-(D)Pro-(D)Arg-(L)Ala-PAM-(D)Pro-(D)Ser-(D)Glu-(D)Arg-Gly-PAM-(D)Pro-(D)Lys-(D)Tyr-(D)Arg-(L)Phe-PAM-(D)Arg-(D)Tyr-(D)Thr-(D)Glu-(L)Ala-PAM-Gly-(D)Trp-(D)His- (D)Arg-(L)Ala-PAM;
fluorescein-(D)Leu-(D)Pro-(D)Glu-(D)Ser-(L)Phe-PAM-Gly-(D)Val-(D)Pro-(D)Arg-(L)Ala-PAM-(D)Pro-(D)Ser-(D)Glu-(D)Arg-Gly-PAM-(D)Pro-(D)Lys-(D)Tyr-(D)Arg-(L)Phe-PAM-(D)Arg-(D)Tyr-(D)Thr-(D)Glu-(L)Ala-PAM-(D)Tyr-(D)Pro-(D)Arg- (D)Glu-Gly-PAM;
fluorescein-(D)Trp-(D)Pro-(D)His-(D)Tyr-(L)Val-PAM-(D)Pro-(D)Ser-Gly-(D)Ser-(L)Ala-PAM-(D)Pro-(D)Ser-(D)Glu-(D)Arg-Gly-PAM-(D)Pro-(D)Lys-(D)Tyr-(D)Arg-(L)Phe-PAM-(D)Pro-Gly-(D)Arg-(D)Trp-Gly-PAM-(D)Tyr-(D)Pro-(D)Arg-(D)Glu- Gly-PAM;
fluorescein-(D)Ser-(D)Pro-(D)His-(D)Glu-(L)Phe-PAM-(D)Pro-(D)Ser-Gly-(D)Ser-(L)Ala-PAM-(D)Thr-(D)Ser-(D)Arg-(D)Glu-(L)Leu-PAM-(D)Pro-(D)Lys-(D)Tyr-(D)Arg-(L)Phe-PAM-(D)Arg-(D)Tyr-(D)Thr-(D)Glu-(L)Ala-PAM-Gly-(D)Trp-(D)His- (D)Arg-(L)Ala-PAM;
fluorescein-(D)Trp-(D)Pro-(D)His-(D)Tyr-(L)Val-PAM-Gly-(D)Val-(D)Pro-(D)Arg-(L)Ala-PAM-Gly-(D)Pro-(D)Arg-(D)Ser-(L)Phe-PAM-(D)Tyr-(D)Thr-(D)Pro-(D)Arg-(L)Ala-PAM-(D)Pro-Gly-(D)Arg-(D)Trp-Gly-PAM-(D)Ser-(D)Pro-(D)Tyr-(D)Leu- (L)Ala-PAM;
fluorescein-(D)Ser-(D)Phe-(D)Pro-(D)Glu-(L)Leu-PAM-Gly-(D)Val-(D)Pro-(D)Arg-(L)Ala-PAM-(D)Pro-(D)Ser-(D)Glu-(D)Arg-Gly-PAM-(D)Pro-(D)Lys-(D)Tyr-(D)Arg-(L)Phe-PAM-(D)Arg-(D)Phe-(D)Pro-(D)Val-Gly-PAM-Gly-(D)Trp-(D)His-(D)Arg- (L)Ala-PAM;
fluorescein-(D)Glu-(D)Pro-(D)Thr-(D)Glu-(L)Leu-PAM-Gly-(D)Val-(D)Pro-(D)Arg-(L)Ala-PAM-(D)Pro-(D)Ser-(D)Glu-(D)Arg-Gly-PAM-(D)Pro-(D)Lys-(D)Tyr-(D)Arg-(L)Phe-PAM-(D)Arg-(D)Tyr-(D)Thr-(D)Glu-(L)Ala-PAM-Gly-(D)Trp-(D)His- (D)Arg-(L)Ala-PAM;
fluorescein-(D)Tyr-(D)His-(D)Pro-(D)Trp-(L)Val-PAM-Gly-(D)Lys-(D)His-(D)Asn-(L)Phe-PAM-(D)Pro-(D)Ser-(D)Glu-(D)Arg-Gly-PAM-(D)Pro-(D)Lys-(D)Tyr-(D)Arg-(L)Phe-PAM-(D)Pro-Gly-(D)Arg-(D)Trp-Gly-PAM-(D)Tyr-(D)Pro-(D)Arg-(D)Glu- Gly-PAM;
fluorescein-(D)Leu-(D)Pro-(D)Glu-(D)Ser-(L)Phe-PAM-(D)Phe-(D)Glu-(D)Lys-(D)Arg-(L)Leu-PAM-(D)Pro-(D)Ser-(D)Glu-(D)Arg-Gly-PAM-(D)Pro-(D)Lys-(D)Tyr-(D)Arg-(L)Phe-PAM-(D)Pro-Gly-(D)Arg-(D)Trp-Gly-PAM-Gly-(D)Trp-(D)His-(D)Arg- (L)Ala-PAM;
fluorescein-(D)Leu-(D)Pro-(D)Glu-(D)Ser-(L)Phe-PAM-Gly-(D)Val-(D)Pro-(D)Arg-(L)Ala-PAM-(D)Lys-(D)Thr-(D)Glu-(D)Ala-(L)Leu-PAM-(D)Tyr-(D)Thr-(D)Pro-(D)Arg-(L)Ala-PAM-(D)Pro-Gly-(D)Arg-(D)Trp-Gly-PAM-Gly-(D)Trp-(D)His-(D)Arg- (L)Ala-PAM;
fluorescein-(D)Ser-(D)Pro-(D)His-(D)Glu-(L)Phe-PAM-Gly-(D)Val-(D)Pro-(D)Arg-(L)Ala-PAM-(D)Pro-(D)Ser-(D)Glu-(D)Arg-Gly-PAM-(D)Pro-(D)Lys-(D)Tyr-(D)Arg-(L)Phe-PAM-(D)Ala-(D)Glu-(D)Leu-(D)Lys-(L)Ala-PAM-Gly-(D)Trp-(D)His- (D)Arg-(L)Ala-PAM; and fluorescein-(D)Trp-(D)Pro-(D)His-(D)Tyr-(L)Val-PAM-(D)Phe-(D)Glu-(D)Lys-(D)Arg-(L)Leu-PAM-(D)Pro-(D)Ser-(D)Glu-(D)Arg-Gly-PAM-(D)Pro-(D)Lys-(D)Tyr-(D)Arg-(L)Phe-PAM-(D)Pro-Gly-(D)Arg-(D)Trp-Gly-PAM-(D)His-(D)Tyr-(D)Ser-(D)Glu- (L)Phe-PAM.

In some embodiments, the biological target is ASGPR and the synthetic compound is at least 70% similar to a compound selected from:
fluorescein-(D)Leu-(D)Pro-(D)Glu-(D)Ser-(L)Phe-PAM-(D)Phe-(D)Glu-(D)Lys-(D)Arg-(L)Leu-PAM-Gly-(D)Pro-(D)Arg-(D)Ser-(L)Phe-PAM-(D)Pro-(D)Lys-(D)Tyr-(D)Arg-(L)Phe-PAM-(D)Arg-(D)Phe-(D)Pro-(D)Val-Gly-PAM-(D)Leu-(D)Pro-(D)Arg- (D)Thr-Gly-PAM;
fluorescein-(D)Leu-(D)-Pro-(D)Glu-(D)Ser-(L)Phe-PAM-(D)Phe-(D)Glu-(D)Lys-(D)Arg-(L)Leu-PAM-Gly-(D)Pro-(D)Arg-(D)Ser-(L)Phe-PAM-(D)Pro-(D)Arg-(D)His-Gly-(L)Leu-PAM-(D)Arg-(D)Phe-(D)Pro-(D)Val-Gly-PAM-(D)Leu-(D)Pro-(D)Arg- (D)Thr-Gly-PAM;
fluorescein-(D)Leu-(D)-Pro-(D)Glu-(D)Ser-(L)Phe-PAM-(D)Phe-(D)Glu-(D)Lys-(D)Arg-(L)Leu-PAM-Gly-(D)Pro-(D)Arg-(D)Ser-(L)Phe-PAM-(D)Tyr-(D)Thr-(D)Pro-(D)Arg-(L)Ala-PAM-(D)Arg-(D)Phe-(D)Pro-(D)Val-Gly-PAM-(D)Leu-(D)Pro-(D)Arg- (D)Thr-Gly-PAM;
fluorescein-(D)Leu-(D)Pro-(D)Glu-(D)Ser-(L)Phe-PAM-(D)Phe-(D)Glu-(D)Lys-(D)Arg-(L)Leu-PAM-(D)His-(D)Ser-(D)Pro-(D)Arg-(L)Val-PAM-(D)Pro-(D)Lys-(D)Tyr-(D)Arg-(L)Phe-PAM-(D)Arg-(D)Phe-(D)Pro-(D)Val-Gly-PAM-(D)Leu-(D)Pro-(D)Arg- (D)Thr-Gly-PAM;
fluorescein-(D)Leu-(D)Pro-(D)Glu-(D)Ser-(L)Phe-PAM-(D)Phe-(D)Glu-(D)Lys-(D)Arg-(L)Leu-PAM-(D)Tyr-(D)Asn-(D)Arg-(D)His-(L)Phe-PAM-(D)Asn-(D)Pro-(D)Leu-(D)His-(L)Leu-PAM-(D)Arg-(D)Phe-(D)Pro-(D)Val-Gly-PAM-(D)Leu-(D)Pro-(D)Arg- (D)Thr-Gly-PAM;
fluorescein-(D)Leu-(D)Pro-(D)Glu-(D)Ser-(L)Phe-PAM-(D)Phe-(D)Glu-(D)Lys-(D)Arg-(L)Leu-PAM-(D)Tyr-(D)Asn-(D)Arg-(D)His-(L)Phe-PAM-(D)Arg-(D)Ser-(D)Glu-(D)Trp-(L)Leu-PAM-(D)Arg-(D)Phe-(D)Pro-(D)Val-Gly-PAM-(D)Leu-(D)Pro-(D)Arg- (D)Thr-Gly-PAM;
fluorescein-(D)Leu-(D)Pro-(D)Glu-(D)Ser-(L)Phe-PAM-(D)Phe-(D)Glu-(D)Lys-(D)Arg-(L)Leu-PAM-(D)Tyr-(D)Asn-(D)Arg-(D)His-(L)Phe-PAM-(D)Tyr-(D)Thr-(D)Pro-(D)Arg-(L)Ala-PAM-(D)Arg-(D)Phe-(D)Pro-(D)Val-Gly-PAM-(D)Leu-(D)Pro-(D)Arg- (D)Thr-Gly-PAM; and fluorescein-(D)Leu-(D)Pro-(D)Glu-(D)Ser-(L)Phe-PAM-Gly-(D)Val-(D)Pro-(D)Arg-(L)Ala-PAM-Gly-(D)Pro-(D)Arg-(D)Ser-(L)Phe-PAM-(D)Pro-(D)Arg-(D)His-Gly-(L)Leu-PAM-(D)Arg-(D)Phe-(D)Pro-(D)Val-Gly-PAM-(D)Leu-(D)Pro-(D)Arg-(D)Thr-Gly- PAM.

In some embodiments the biological target is IL-6 and the synthetic compound is at least 70% similar to a compound selected from:
fluorescein-(D)Tyr-(D)Leu-(L)Leu-PAM-(D)Tyr-(D)Ser-(L)Val-PAM-(D)Arg-(D)Ser-(L)Phe-PAM-(D)Trp-(D)Arg-(L)Leu-PAM-(D)Trp-(D)Arg-(L)Val-PAM-(D)Arg-(D)Tyr-(L)Leu-PAM-(D)Glu-(D)Tyr-(L)Val-PAM-(D)Lys-(D)Phe-(L)Phe-PAM-(D)Tyr- (D)Arg-(L)Phe-PAM;
fluorescein-(D)His-(D)Leu-(L)Phe-PAM-(D)Ala-(D)Ala-(L)Phe-PAM-(D)Pro-(D)Lys-(L)Ala-PAM-(D)Phe-(D)Arg-(L)Ala-PAM-(D)Ala-(D)Lys-(L)Phe-PAM-(D)Phe-(D)Thr-(L)Ala-PAM-(D)Lys-(D)Asn-(L)Leu-PAM-(D)Thr-(D)His-(L)Leu-PAM-(D)Phe- (D)Lys-Gly-PAM;
fluorescein-(D)Arg-(D)Trp-(L)Phe-PAM-(D)Glu-(D)Ser-(L)Phe-PAM-(D)Arg-(D)Ser-(L)Phe-PAM-(D)Trp-(D)Arg-(L)Leu-PAM-Gly-(D)Ala-(L)Val-PAM-(D)Asn-(D)Arg-(L)Phe-PAM-(D)Ala-(D)Ser-(L)Phe-PAM-(D)Pro-(D)Arg-(L)Ala-PAM-(D)Phe-(D)Lys- Gly-PAM;
fluorescein-(D)Arg-(D)Trp-(L)Phe-PAM-(D)Leu-(D)Arg-(L)Leu-PAM-(D)His-(D)Tyr-(L)Phe-PAM-(D)Trp-(D)Arg-(L)Leu-PAM-(D)His-(D)Lys-(L)Leu-PAM-(D)Ala-(D)Tyr-Gly-PAM-(D)His-(D)Glu-(L)Leu-PAM-(D)Pro-(D)Arg-(L)Ala-PAM-(D)Phe-(D)Lys- Gly-PAM;
fluorescein-(D)Ser-(D)Phe-(L)Phe-PAM-Gly-(D)Arg-(L)Leu-PAM-(D)Leu-(D)Lys-(L)Leu-PAM-(D)Trp-(D)Arg-(L)Leu-PAM-(D)Ala-(D)Lys-(L)Phe-PAM-(D)His-(D)Lys-(L)Phe-PAM-(D)Ala-(D)Arg-(L)Leu-PAM-(D)Thr-(D)His-(L)Leu-PAM-(D)Phe-(D)Arg- Gly-PAM;
fluorescein-(D)Tyr-(D)Leu-(L)Leu-PAM-Gly-(D)Arg-(L)Leu-PAM-(D)Arg-(D)Ser-(L)Phe-PAM-(D)Phe-(D)Arg-(L)Ala-PAM-(D)Glu-(D)Lys-(L)Phe-PAM-(D)Glu-(D)Arg-(L)Leu-PAM-(D)Ala-(D)Arg-(L)Leu-PAM-(D)Pro-(D)Arg-(L)Ala-PAM-(D)Tyr-(D)Arg- (L)Phe-PAM;
fluorescein-(D)Tyr-(D)Leu-(L)Leu-PAM-Gly-(D)Arg-(L)Leu-PAM-(D)Arg-(D)Ser-(L)Phe-PAM-(D)Pro-(D)Arg-(L)Val-PAM-(D)Trp-(D)Arg-(L)Val-PAM-(D)Asn-(D)Arg-(L)Phe-PAM-(D)Glu-(D)Tyr-(L)Val-PAM-(D)Pro-(D)Arg-(L)Ala-PAM-(D)Tyr-(D)Arg- (L)Phe-PAM; and fluorescein-(D)Thr-(D)Leu-(L)Phe-PAM-Gly-(D)Arg-(L)Leu-PAM-(D)Arg-(D)Ser-(L)Phe-PAM-(D)Pro-(D)Arg-(L)Val-PAM-(D)Trp-(D)Arg-(L)Val-PAM-(D)Asn-(D)Arg-(L)Phe-PAM-(D)Glu-(D)Tyr-(L)Val-PAM-(D)Pro-(D)Arg-Ala-PAM-Tyr-Arg-Phe-PAM.

In some embodiments, the biological target is IL-6R and the synthetic compound is at least 70% similar to a compound selected from:
fluorescein-(D)Arg-(D)Trp-(L)Phe-PAM-(D)Pro-(D)Asn-(L)Val-PAM-(D)Asn-(D)Thr-(L)Leu-PAM-(D)Lys-(D)Glu-(L)Leu-PAM-(D)Glu-(D)Lys-(L)Phe-PAM-(D)Glu-(D)Trp-(L)Ala-PAM-(D)Ala-(D)Arg-(L)Leu-PAM-(D)Glu-(D)Arg-(L)Val-PAM-(D)Phe- (D)Lys-Gly-PAM;
fluorescein-(D)Tyr-(D)Leu-(L)Leu-PAM-(D)Glu-(D)Ser-(L)Phe-PAM-(D)Asn-(D)Thr-(L)Leu-PAM-(D)Lys-(D)Glu-(L)Leu-PAM-(D)Glu-(D)Lys-(L)Phe-PAM-(D)Glu-(D)Arg-(L)Leu-PAM-(D)Ala-(D)Arg-(L)Leu-PAM-(D)Trp-(D)Lys-(L)Leu-PAM-(D)Phe- (D)Lys-Gly-PAM;
fluorescein-(D)Arg-(D)Trp-(L)Phe-PAM-(D)Glu-(D)Ser-(L)Phe-PAM-(D)Asn-(D)Thr-(L)Leu-PAM-(D)Tyr-(D)Asn-(L)Phe-PAM-(D)Tyr-(D)Lys-(L)Ala-PAM-(D)Glu-(D)Arg-(L)Leu-PAM-(D)Ala-(D)Arg-(L)Leu-PAM-(D)Glu-(D)Arg-(L)Val-PAM-(D)Phe- (D)Lys-Gly-PAM;
fluorescein-(D)Asn-(D)Val-(L)Phe-PAM-(D)Glu-(D)Ser-(L)Phe-PAM-(D)Arg-(D)Ser-(L)Phe-PAM-(D)Lys-(D)Glu-(L)Leu-PAM-(D)Trp-(D)Arg-(L)Val-PAM-(D)Pro-(D)Glu-(L)Phe-PAM-(D)Ala-(D)Arg-(L)Leu-PAM-(D)Pro-(D)Arg-(L)Ala-PAM-(D)Phe- (D)Arg-Gly-PAM;
fluorescein-(D)Arg-(D)Trp-(L)Phe-PAM-(D)Leu-(D)Arg-(L)Leu-PAM-(D)Asn-(D)Thr-(L)Leu-PAM-(D)Pro-(D)Arg-(L)Val-PAM-Gly-(D)Ala-(L)Val-PAM-(D)Glu-(D)Arg-(L)Leu-PAM-(D)Ala-(D)Arg-(L)Leu-PAM-(D)Pro-(D)Arg-(L)Ala-PAM-(D)Phe-(D)Arg- Gly-PAM;
fluorescein-(D)Arg-(D)Trp-(L)Phe-PAM-(D)Pro-(D)Asn-(L)Val-PAM-(D)Pro-(D)Lys-(L)Ala-PAM-(D)Trp-(D)Arg-(L)Leu-PAM-(D)Ala-(D)Lys-(L)Phe-PAM-(D)Pro-(D)Glu-(L)Phe-PAM-(D)Ala-(D)Arg-(L)Leu-PAM-(D)Trp-(D)Lys-(L)Leu-PAM-(D)Phe- (D)Lys-Gly-PAM;
fluorescein-(D)Asn-(D)Val-(L)Phe-PAM-(D)Tyr-(D)Ser-(L)Val-PAM-(D)Arg-(D)Ser-(L)Phe-PAM-(D)Phe-(D)Arg-(L)Ala-PAM-(D)Tyr-(D)Lys-(L)Ala-PAM-(D)Glu-(D)Arg-(L)Leu-PAM-(D)Ala-(D)Arg-(L)Leu-PAM-(D)Pro-(D)Arg-(L)Ala-PAM-(D)Phe- (D)Lys-Gly-PAM; and fluorescein-(D)Arg-(D)Trp-(L)Phe-PAM-(D)Leu-(D)Arg-(L)Leu-PAM-(D)Phe-(D)Glu-(L)Val-PAM-(D)Lys-(D)Glu-(L)Leu-PAM-(D)Ala-(D)Lys-(L)Phe-PAM-(D)Asn-(D)Arg-(L)Phe-PAM-(D)Ala-(D)Arg-(L)Leu-PAM-(D)Trp-(D)Lys-(L)Leu-PAM-(D)Ala-(D)Val- (L)Leu-PAM.

In some embodiments, the biological target is TNFα and the synthetic compound is at least 70% similar to a compound selected from:
fluorescein-(D)Lys-(D)Val-(L)Leu-PAM-(D)Ala-(D)Ala-(L)Phe-PAM-(D)Leu-(D)Lys-(L)Leu-PAM-(D)Phe-(D)Lys-(L)Leu-PAM-(D)Trp-(D)Arg-(L)Val-PAM-(D)Ser-(D)Ser-(L)Phe-PAM-(D)His-(D)Glu-(L)Leu-PAM-(D)Phe-(D)Lys-(L)Phe-PAM-(D)Thr- (D)Glu-(L)Leu-PAM;
fluorescein-(D)Thr-(D)Leu-(L)Phe-PAM-(D)Leu-(D)Arg-(L)Leu-PAM-(D)Leu-(D)Lys-(L)Leu-PAM-(D)Trp-(D)Arg-(L)Leu-PAM-(D)Ala-(D)Lys-(L)Phe-PAM-(D)Glu-(D)Ser-(L)Val-PAM-(D)Pro-(D)Val-Gly-PAM-(D)Glu-(D)Tyr-(L)Phe-PAM-(D)Tyr-(D)Arg- (L)Phe-PAM;
fluorescein-(D)Lys-(D)Val-(L)Leu-PAM-Gly-(D)Arg-(L)Leu-PAM-(D)Phe-(D)Glu-(L)Val-PAM-(D)Pro-(D)Arg-(L)Val-PAM-(D)Ala-(D)Lys-(L)Phe-PAM-(D)Glu-(D)Ser-(L)Val-PAM-(D)Pro-(D)Val-Gly-PAM-(D)Thr-(D)His-(L)Leu-PAM-(D)Tyr-(D)Arg- (L)Phe-PAM;
fluorescein-(D)Lys-(D)Val-(L)Leu-PAM-(D)Leu-(D)Arg-(L)Leu-PAM-(D)Phe-(D)Glu-(L)Val-PAM-(D)Phe-(D)Arg-(L)Ala-PAM-(D)Ala-(D)Lys-(L)Phe-PAM-(D)Phe-(D)Thr-(L)Ala-PAM-(D)Glu-(D)Tyr-(L)Val-PAM-Gly-(D)Glu-(L)Leu-PAM-(D)Trp-(D)Glu- (L)Val-PAM;
fluorescein-(D)Lys-(D)Val-(L)Leu-PAM-(D)Leu-(D)Arg-(L)Leu-PAM-(D)Phe-(D)Glu-(L)Val-PAM-(D)Trp-(D)Arg-(L)Leu-PAM-(D)Ala-(D)Lys-(L)Phe-PAM-(D)Arg-(D)Tyr-(L)Leu-PAM-(D)Pro-(D)Val-Gly-PAM-(D)Lys-(D)Phe-(L)Phe-PAM-(D)His-(D)Tyr- (L)Val-PAM;
fluorescein-(D)Tyr-(D)Leu-(L)Leu-PAM-(D)Leu-(D)Arg-(L)Leu-PAM-(D)Leu-(D)Lys-(L)Leu-PAM-(D)Phe-(D)Arg-(L)Ala-PAM-(D)Ala-(D)Lys-(L)Phe-PAM-(D)Asn-(D)Arg-(L)Phe-PAM-(D)Ala-(D)Arg-(L)Leu-PAM-(D)Trp-(D)Lys-(L)Leu-PAM-(D)Pro- (D)Ala-(L)Ala-PAM;
fluorescein-(D)Lys-(D)Val-(L)Leu-PAM-(D)Leu-(D)Arg-(L)Leu-PAM-(D)Arg-(D)Val-(L)Leu-PAM-(D)Phe-(D)Arg-(L)Ala-PAM-(D)Tyr-(D)Lys-(L)Ala-PAM-(D)Glu-(D)Arg-(L)Leu-PAM-(D)Pro-(D)Val-Gly-PAM-(D)Lys-(D)Phe-(L)Phe-PAM-(D)His-(D)Glu- (L)Val-PAM;
fluorescein-(D)Lys-(D)Val-(L)Leu-PAM-(D)Leu-(D)Arg-(L)Leu-PAM-(D)Phe-(D)Glu-(L)Val-PAM-(D)Phe-(D)Lys-(L)Leu-PAM-(D)Pro-(D)Glu-(L)Leu-PAM-(D)Phe-(D)Thr-(L)Ala-PAM-(D)His-(D)Glu-(L)Leu-PAM-(D)Glu-(D)Tyr-(L)Phe-PAM-(D)Thr- (D)Ser-(L)Val-PAM;
fluorescein-(D)Lys-(D)Val-(L)Leu-PAM-(D)Leu-(D)Arg-(L)Leu-PAM-(D)Arg-(D)Val-(L)Leu-PAM-(D)Phe-(D)Arg-(L)Ala-PAM-(D)Ala-(D)Lys-(L)Phe-PAM-(D)Asn-(D)Arg-(L)Phe-PAM-(D)Pro-(D)Val-Gly-PAM-(D)Glu-(D)Arg-(L)Val-PAM-(D)Tyr-(D)Arg- (L)Phe-PAM;
fluorescein-(D)Tyr-(D)Leu-(L)Leu-PAM-Gly-(D)Arg-(L)Leu-PAM-(D)Phe-(D)Glu-(L)Val-PAM-(D)Trp-(D)Arg-(L)Leu-PAM-(D)Trp-(D)Arg-(L)Val-PAM-(D)Arg-(D)Tyr-(L)Leu-PAM-(D)Pro-Gly-(L)Val-PAM-(D)His-(D)Ser-(L)Leu-PAM-(D)Phe-(D)Lys-Gly- PAM;
fluorescein-(D)Thr-(D)Leu-(L)Phe-PAM-(D)Leu-(D)Arg-(L)Leu-PAM-(D)Phe-(D)Glu-(L)Val-PAM-(D)Phe-(D)Arg-(L)Ala-PAM-(D)Ala-(D)Lys-(L)Phe-PAM-(D)Arg-(D)Tyr-(L)Leu-PAM-(D)Pro-Gly-(L)Val-PAM-(D)Trp-(D)Lys-(L)Leu-PAM-(D)Tyr-(D)Arg- (L)Phe-PAM;
fluorescein-(D)Thr-(D)Leu-(L)Phe-PAM-(D)Leu-(D)Arg-(L)Leu-PAM-(D)Phe-(D)Glu-(L)Val-PAM-(D)Phe-(D)Lys-(L)Leu-PAM-(D)Pro-(D)Glu-(L)Leu-PAM-(D)Pro-(D)Glu-(L)Phe-PAM-(D)His-(D)Glu-(L)Leu-PAM-(D)Pro-(D)Arg-(L)Ala-PAM-(D)His- (D)Tyr-(L)Val-PAM;
fluorescein-(D)Lys-(D)Val-(L)Leu-PAM-(D)Leu-(D)Arg-(L)Leu-PAM-(D)Phe-(D)Glu-(L)Val-PAM-(D)Phe-(D)Arg-(L)Ala-PAM-(D)Tyr-(D)Lys-(L)Ala-PAM-(D)Glu-(D)Ser-(L)Val-PAM-(D)Pro-(D)Val-Gly-PAM-(D)Pro-(D)Arg-(L)Ala-PAM-(D)Phe-(D)Lys- Gly-PAM;
fluorescein-(D)Tyr-(D)Leu-(L)Leu-PAM-(D)Leu-(D)Arg-(L)Leu-PAM-(D)Phe-(D)Glu-(L)Val-PAM-(D)Pro-(D)Arg-(L)Val-PAM-(D)Trp-(D)Arg-(L)Val-PAM-(D)Arg-(D)Tyr-(L)Leu-PAM-(D)Glu-(D)Val-(L)Val-PAM-(D)Thr-(D)His-(L)Leu-PAM-(D)Tyr- (D)Arg-(L)Phe-PAM;
fluorescein-(D)Lys-(D)Val-(L)Leu-PAM-(D)Leu-(D)Arg-(L)Leu-PAM-(D)Arg-(D)Val-(L)Leu-PAM-(D)Phe-(D)Lys-(L)Leu-PAM-(D)Ala-(D)Lys-(L)Phe-PAM-(D)Phe-(D)Thr-(L)Ala-PAM-(D)Pro-Gly-(L)Val-PAM-(D)Lys-(D)Phe-(L)Phe-PAM-(D)Ala-(D)Val- (L)Leu-PAM;
fluorescein-(D)Lys-(D)Val-(L)Leu-PAM-(D)Trp-(D)His-(L)Ala-PAM-(D)Phe-(D)Glu-(L)Val-PAM-(D)Trp-(D)Arg-(L)Leu-PAM-(D)Tyr-(D)Lys-(L)Ala-PAM-(D)Asn-(D)Arg-(L)Phe-PAM-(D)Pro-(D)Val-Gly-PAM-(D)Pro-(D)Arg-(L)Ala-PAM-(D)Ala-(D)Lys- Gly-PAM;
fluorescein-(D)Lys-(D)Val-(L)Leu-PAM-Gly-(D)Arg-(L)Leu-PAM-(D)Arg-(D)Val-(L)Leu-PAM-(D)Trp-(D)Arg-(L)Leu-PAM-(D)Trp-(D)Arg-(L)Val-PAM-(D)Ser-(D)Ser-(L)Phe-PAM-(D)Ala-(D)Ser-(L)Phe-PAM-(D)Trp-(D)Lys-(L)Leu-PAM-(D)Tyr-(D)Arg- (L)Phe-PAM;
fluorescein-(D)Lys-(D)Val-(L)Leu-PAM-(D)Ala-(D)Ala-(L)Phe-PAM-(D)Phe-(D)Glu-(L)Val-PAM-(D)Pro-(D)Arg-(L)Val-PAM-(D)Ala-Gly-(L)Phe-PAM-(D)Glu-(D)Arg-(L)Leu-PAM-(D)Pro-(D)Val-Gly-PAM-(D)Trp-(D)Lys-(L)Leu-PAM-(D)Pro-(D)Ala- (L)Ala-PAM;
fluorescein-(D)Lys-(D)Val-(L)Leu-PAM-(D)Leu-(D)Arg-(L)Leu-PAM-(D)Phe-(D)Glu-(L)Val-PAM-(D)Phe-(D)Arg-(L)Ala-PAM-(D)Ala-(D)Lys-(L)Phe-PAM-(D)Asn-(D)Arg-(L)Phe-PAM-(D)Pro-(D)Val-Gly-PAM-(D)Trp-(D)Lys-(L)Leu-PAM-(D)Tyr-(D)Arg- (L)Phe-PAM;
fluorescein-(D)Lys-(D)Val-(L)Leu-PAM-Gly-(D)Arg-(L)Leu-PAM-(D)Leu-(D)Lys-(L)Leu-PAM-(D)Pro-(D)Arg-(L)Val-PAM-(D)Ala-(D)Lys-(L)Phe-PAM-(D)Glu-(D)Trp-(L)Ala-PAM-(D)His-(D)Glu-(L)Leu-PAM-(D)Glu-(D)Arg-(L)Val-PAM-(D)Thr-(D)Ser- (L)Val-PAM;
fluorescein-(D)Ser-(D)Val-(L)Leu-PAM-Gly-(D)Arg-(L)Leu-PAM-(D)Phe-(D)Glu-(L)Val-PAM-(D)Phe-(D)Lys-(L)Leu-PAM-(D)Tyr-(D)Lys-(L)Ala-PAM-(D)Glu-(D)Trp-(L)Ala-PAM-(D)Ala-(D)Arg-(L)Leu-PAM-(D)Glu-(D)Tyr-(L)Phe-PAM-(D)Tyr-(D)Arg- (L)Phe-PAM;
fluorescein-(D)Tyr-(D)Leu-(L)Leu-PAM-(D)Leu-(D)Arg-(L)Leu-PAM-(D)Leu-(D)Lys-(L)Leu-PAM-(D)Phe-(D)Arg-(L)Ala-PAM-(D)Ala-(D)Lys-(L)Phe-PAM-(D)Phe-(D)Thr-(L)Ala-PAM-(D)Pro-(D)Val-Gly-PAM-(D)Thr-(D)His-(L)Leu-PAM-(D)Thr-(D)Ser- (L)Val-PAM; and fluorescein-(D)Lys-(D)Val-(L)Leu-PAM-Gly-(D)Arg-(L)Leu-PAM-(D)Phe-(D)Glu-(L)Val-PAM-(D)Phe-(D)Arg-(L)Ala-PAM-(D)Trp-(D)Arg-(L)Val-PAM-(D)Glu-(D)Arg-(L)Leu-PAM-(D)His-(D)Glu-(L)Leu-PAM-(D)Lys-(D)Phe-(L)Phe-PAM-(D)Tyr-(D)Arg- (L)Phe-PAM.

Although the above describes various example synthetic compounds with a fluorescein in the sequence of the compound, embodiments are not so limited and may include the above-described synthetic compounds specific to the different targets without the fluorescein in the sequence.

### EXPERIMENTAL EMBODIMENTS

A number of experimental embodiments were conducted to generate assays including binding assays, and screening the assays to identify different synthetic compounds that are specific to biological targets of K-Ras, IL-6, TNFα, IL-6R, and ASGPR. A number of experimental embodiments were conducted to assess the structures of the different synthetic compounds and to characterize the compounds.

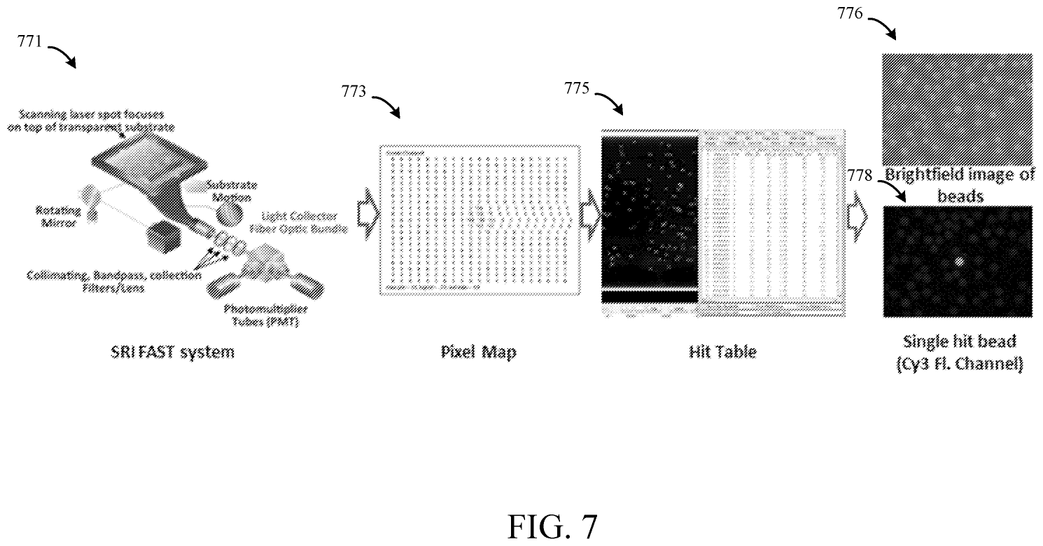

FIG. 7 illustrates an example system used to screen a library of a plurality of synthetic compounds, in accordance with the present disclosure. More particularly, FIG. 7 illustrates an example FAST system 771 that can screen around five million synthetic compound per minute. The FAST system 771 includes a fiber optic scanner, which includes a fiber optic bundle array, a laser, and imaging circuitry (e.g., camera). The FAST system 771 uses rapid laser scanning with sensitive photomultiplier tube (PMT) fluorescence emission detection to rapidly generate a pixel map, at 773, indicating the position of fluorescently labeled beads. Analysis of the pixel map generates a hit table, at 775, with cartesian coordinates and multiple calculated fluorescence metrics to detect bead hits with high sensitivity and specificity. The coordinates of the hits are transferred to other microscopy systems for additional multi-wavelength imaging analysis or bead extraction, at 776 and 778.

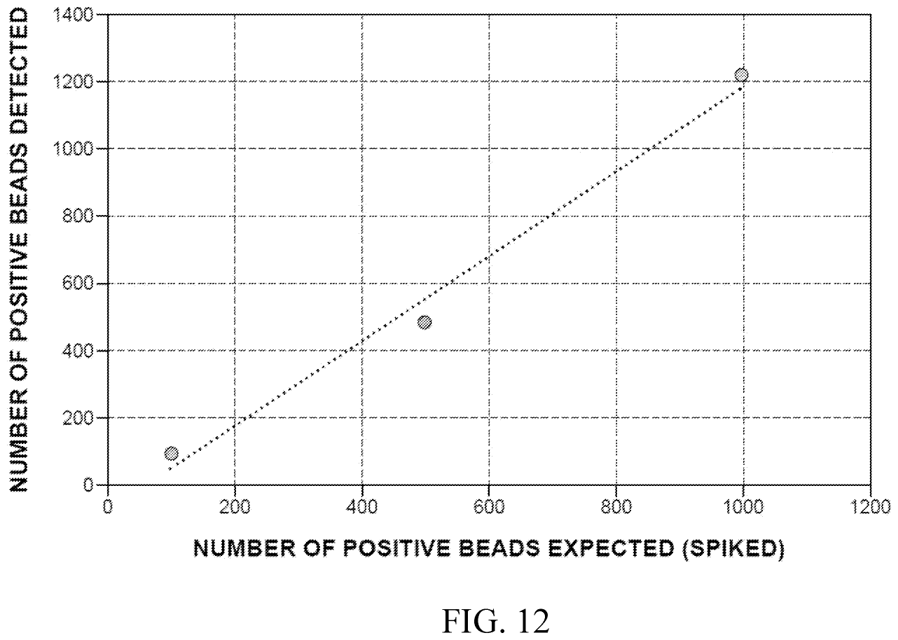

In some experiments, cells preincubated with fluorescently labeled cell surface markers were plated as a monolayer on 108×76-mm glass slides, which were scanned by excitation with a 488 nm laser by the FAST system 771. Emitted fluorescence was collected through a fiber-optic bundle, and the collected light was passed through a bandpass filter and analyzed by a photomultiplier tube to measure emission at 520 nm (e.g., green) and 580 nm (e.g., red/orange) to eliminate true negatives due to auto-fluorescence (see below). Cartesian coordinates of fluorescently labeled objects were located on a pixel map 773, along with fluorescent intensity measurements at the two emission wavelengths. In this well-free assay format, FAST can routinely identify the location of single rare cells in a milieu of 25 million white cells in a 1-minute scan with an around 8-μm resolution. In optimizing the FAST system 771 for bead screening, the scanning process of FAST was modified by plating the beads at a lower density than cells due to the propensity of the beads to aggregate and the need to extract the beads post-analysis for sequencing. Empirical optimization of bead plating density revealed that 10-μm diameter TentaGel™ beads plated with a density of 5 million beads per plate gave a relatively well-dispersed monolayer enabling automated analysis and bead picking for down-stream processing. Similarly, 20-μm beads were optimally plated at a density of 2.5 million beads per plate. Detection sensitivity was assessed by spiking biotin-labeled beads into a pool of underivatized beads and incubating with Alexa Fluor 555-labeled streptavidin for 1 hour before plating. The FAST-screening process gave a detection sensitivity of over 99.99% (see, e.g., Table 2 and FIG. 12).

In considering the application of FAST to bead-based screening, the following considerations were made to obtain efficient fluorescence-based screening of TentaGel™ OBOC libraries. The first is that the auto-fluorescence of TentaGel™ beads leads to low signal-to-noise ratios and complicates the identification of hits. The FAST-screening approach uses several strategies to overcome the low signal-to-noise ratios due to bead auto-fluorescence based on optical properties of the TentaGel™ resin. As the TentaGel™ auto-fluorescence is highly significant in the FITC (fluorescein isothiocyanate) channel and the fluorescence intensity diminishes as its wavelength shift increases and auto-fluorescence intensifies with increasing bead size, the functionalized beads with different chemistries have various levels of auto-fluorescence and are slightly higher than the auto-fluorescence of unfunctionalized beads. As noted above, the size of beads used for the library construction in various experimental embodiments was 10-20 um in diameter (e.g., comparable to a mammalian-cell size) and the auto-fluorescence was significantly lower than the beads commonly used in other OBOC libraries (e.g., 90-300 um). Secondly, a particular fluorophore (Alexa-fluor 555 or CF555, yellow/orange) for target probes was used in conjunction with a wavelength comparison technique engineered in the FAST system to eliminate the effects of fluorescence from auto-fluorescing particles. The technique involves measuring emissions at two different wavelengths, one at the target emission wavelength (580 nm) and the other at 520 nm, a wavelength intermediate between the target emission wavelength and the laser excitation (488 nm). Because auto-fluorescence is typically more intense at wavelengths closer to the excitation, the ratio of the intermediate wavelength intensity to the target wavelength intensity is greater than one for unlabeled beads, while for labeled beads the ratio is less than one. A software filter used this ratio to eliminate the auto-fluorescing beads. The software filter also screened for and eliminated fluorescence-positive objects originating from dye aggregates and bead fragments by filtering for object size and relative brightness. The negative controls were set in parallel for each screen. Negative controls include fluorescence cutoffs determined from unfunctionalized naked TentaGel™ beads that have gone through the assay staining process with labeled probe, and a portion of the library beads taken through the assay staining protocol without the probe. As part of the comprehensive filter settings, the fluorescence intensity cutoff threshold is set to eliminate selection of auto-fluorescent beads due to the TentaGel™ or library background (e.g., filter threshold setting details are described in Tables 6-11). With this filter, 99.8% of the located objects on the glass slide were eliminated as true negatives and are not selected for further analysis. With these filters, the typical hit number from a FAST scan is around 100-400 identified as corresponding coordinate locations on the glass slide from a sample containing 2.5 million 20-um beads or 5 million 10-um beads.

The hits identified by the FAST primary scan, such as at 775, were then automatically imaged and analyzed by high resolution Automated Digital Microscopy (ADM) on a CellCelector (ALS Automated Lab Solution GmbH) using bright-field, target Alexa Fluor 555 (AF555) or CF555 and counter target AF647/Cy5 channels. Hit beads were quality control/quality assurance (QC/QA) reviewed based on morphology and fluorescence staining data. Damaged beads, beads with irregular shape, size or staining pattern, and hit beads located within a large aggregate and impossible to exact were excluded. The Mean Fluorescence Intensity (MFI) was then measured for all hits that pass initial QC/QA. All “true positive” (TP) hits were ranked based on MFI intensity and/or ratio of selected channels and generally the top around 50 beads from the initially 10-400 FAST hits were selected and isolated for sequencing and hit confirmation by resynthesis and K_{D} characterization.

Another consideration with OBOC libraries is that during on-bead screening the signal strengths (e.g., fluorescence intensities) do not always correlate with the potency of the ligands on these beads. One of the contributing factors to this is that commercial resins typically used for library synthesis have high ligand loading (e.g., 90-um TentaGel resin with a loading capacity of 0.3 mmol/g has a ligand density of around 100 mM) which is used to provide a sufficient amount of material for subsequent hit identification, but can cause false positives and screening biases due to the unintended multidentate interaction with high ligand density on the beads. In various experimental embodiments, the avidity effects were minimized by the use of smaller beads with less ligand loading. This also allowed for the use lower probe concentrations in screening. For each screen, the probes are pre-titrated to identify the minimum probe concentrations that achieve optimal signal-to-noise ratio and hit numbers (e.g., Titration and optimization details are described in Tables 6-11). These strategies increase the probability of identifying the most active hit(s) while minimizing false positives.

In some experiments, the 10-μm diameter beads at around 0.2 mmol/g loading typically carry around 100 fmols of a synthetic compound that is readily detectable by high-resolution LC-MS (e.g., Table 1). Using ptych design sequencing, polymer libraries can be synthesized on much smaller diameter beads to create much larger libraries. Combined with the FAST system 771, this allows for screening and hit identification of much larger synthetic bead-based polymer libraries than previously possible.

In a validation experiment, to determine the reliability of sequences from individual beads, a set of 90 individual 10-μm beads, each containing one of four possible unique sequences, were mixed and then picked from a plate and cleaved and sequenced. The full correct sequence were obtained for 82 of the picked beads (91%) (see Tables 3A-3D). There were no incorrect sequence assignments in any of the validation samples in which ptychs were detected. The samples that did not yield an identifiable sequence also did not yield any ptych assignments, indicating the beads were likely not deposited correctly in the vial or were otherwise lost during automated sample processing, which is a factor in microscale handling efficiency and hit confirmation rates.

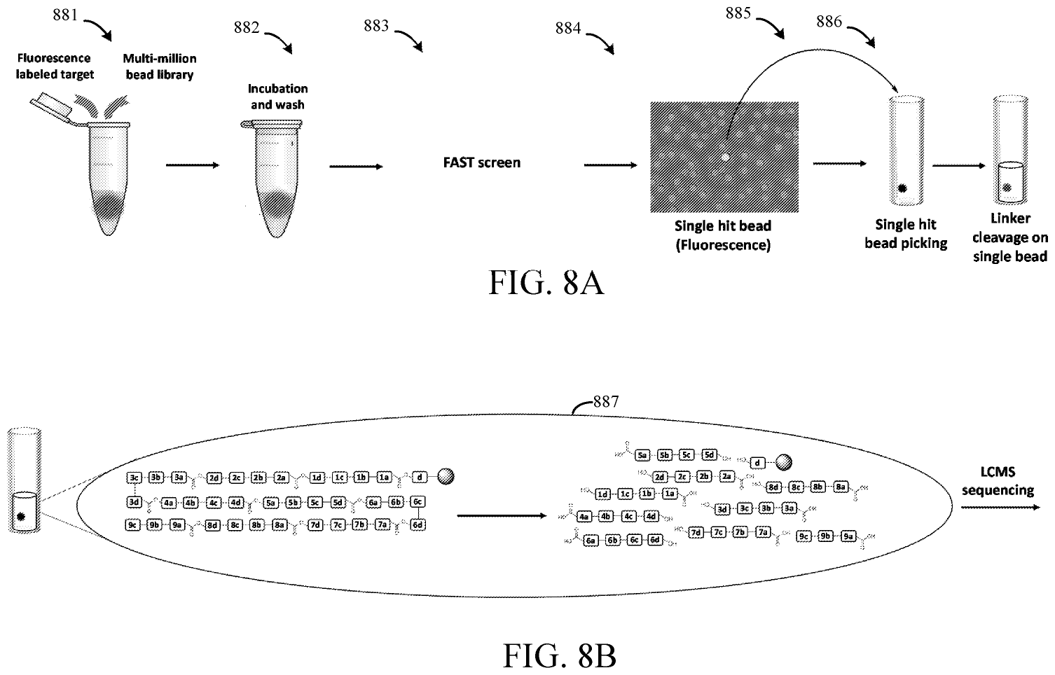

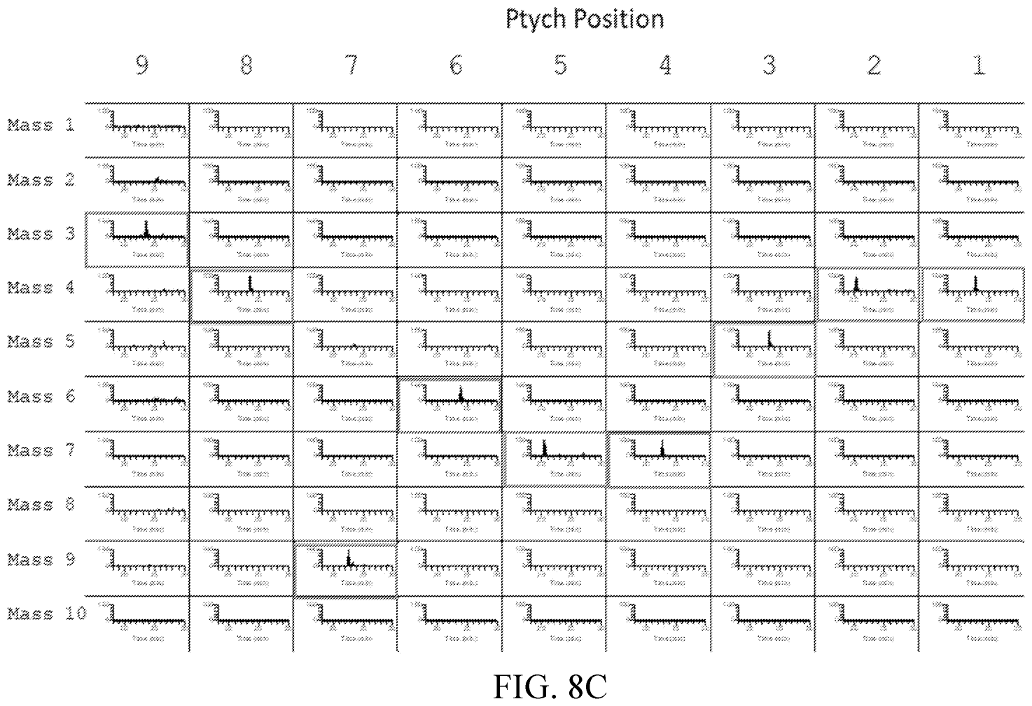

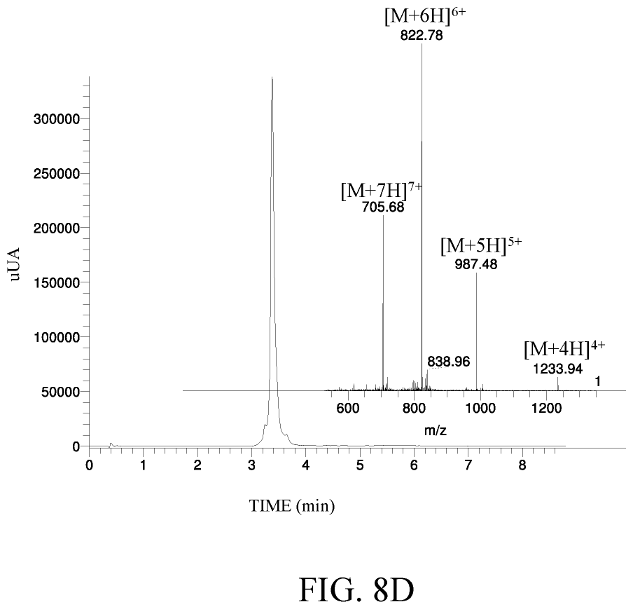

FIGS. 8A-8D illustrate an example screening process of library of a plurality of synthetic compounds, in accordance with the present disclosure. For example, the library of synthetic compounds was screened using the FAST system and designed using a ptych library design. Assay development involved a preliminary titration screen using varying concentrations of targets against the library and naked control beads (see Tables 6-10) to minimize the effects of auto-fluorescence, as described above, while maximizing signal-to-noise. Based on these results, target screening concentrations and the background MFI threshold were selected for optimum hit fluorescent signals relative to background (see Table 11).

As shown by FIG. 8A, the library was incubated with a fluorescently labeled target in 50% Odyssey buffer and 0.5% CHAPS blocking buffer to screen out non-specific binding and was followed by a sequence of washes (see Table 11), at 881 and 882. The beads were then plated as a monolayer on glass slides and FAST screened to identify positive hits defined as fluorescently labeled beads that indicates binding to the target, at 883 in FIG. 8A. The plate and the hit location data from the FAST system were transferred to an automated fluorescence microscope and picking robot (ALS CellCelector) for preliminary hit quality control, at 884. Confirmed hit beads were individually transferred into vials, at 885, and treated with cleavage solution to hydrolyze the backbone esters yielding a mixture of, in this case, tetraptychs which were sequenced by LCMS, at 886 (see also FIG. 8B-8C).

As shown at 887 in FIG. 8B, during sequencing each synthetic compound (e.g., polymer) was subjected to a chemical cleavage reaction in which the linkers are cleaved to generate a mixture of the ptych fragments. More particularly, upon treatment with ammonium hydroxide, all the esters were hydrolyzed to yield a mixture of the different tetraptychs of a single sequence in each vial.

The hit sequences were re-synthesized and purified by preparative high-performance LC (HPLC) for hit confirmation and further testing. FIG. 8C illustrates analysis of ptych fragment masses allows for reconstruction of the ptychs into a specific sequence based on the library design. FIG. 8D illustrates an example MS chromatogram of a purified full-length 36-mer (9 tetraptych) polymer of which the sequence analysis is shown in FIG. 8C. The compound hit was characterized by LC-MS (ESI); observed: 4931.9±0.9 Da; calculated: 4932.5 Da.

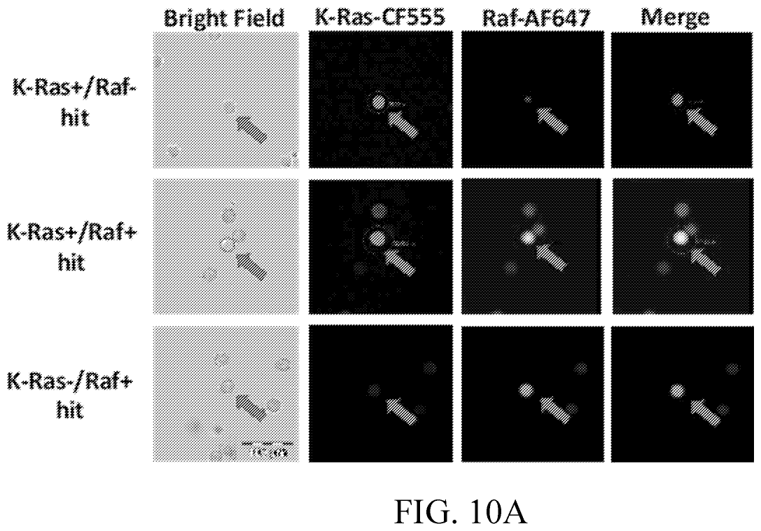

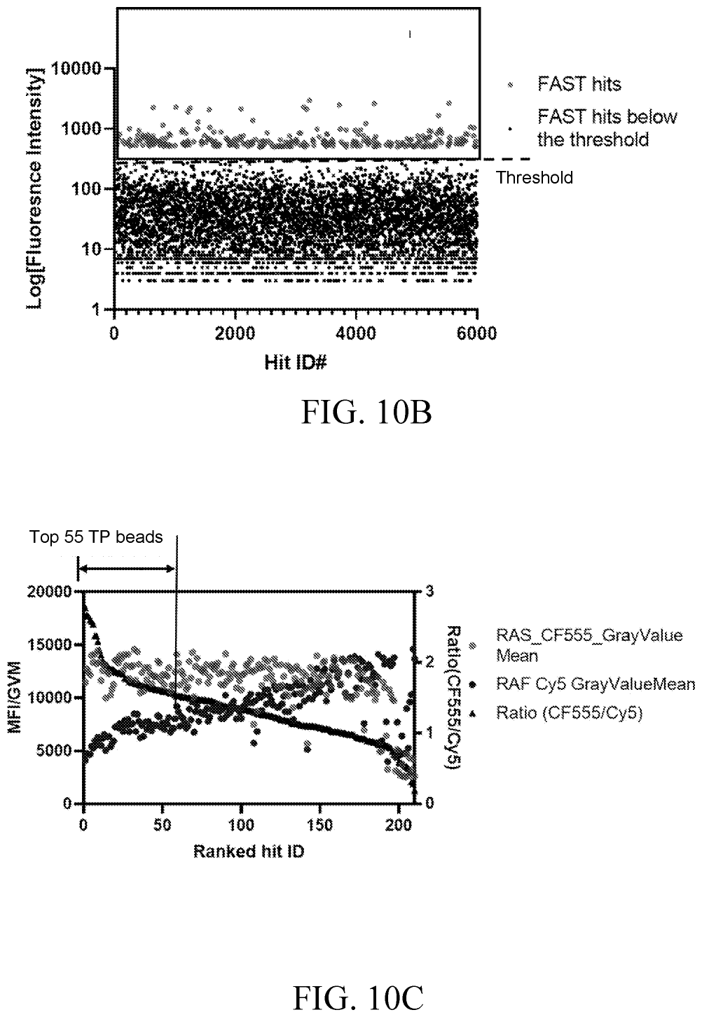

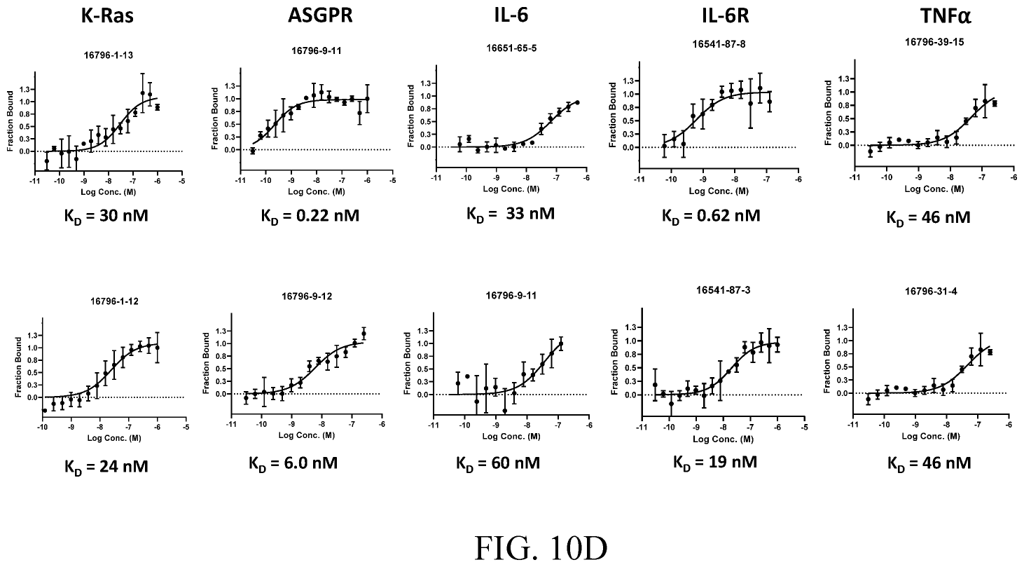

A preliminary study of hits showed switching the backbone ester bonds to amides had only minor effects on measured binding affinities (see Table 12), and as this greatly improves compound stability and simplifies hit resynthesis, all hits were prepared as the full backbone amide analogs. The resynthesized hit binding affinities for their respective targets was measured using microscale thermophoresis (MST) (see FIG. 10D). Binding was confirmed in the majority of all backbone amide resynthesized hits with binding affinities in the nanomolar to sub-nanomolar range which indicated that switching out backbone esters for amide bonds generally has minimal effects on hit confirmation for these NNP designs.

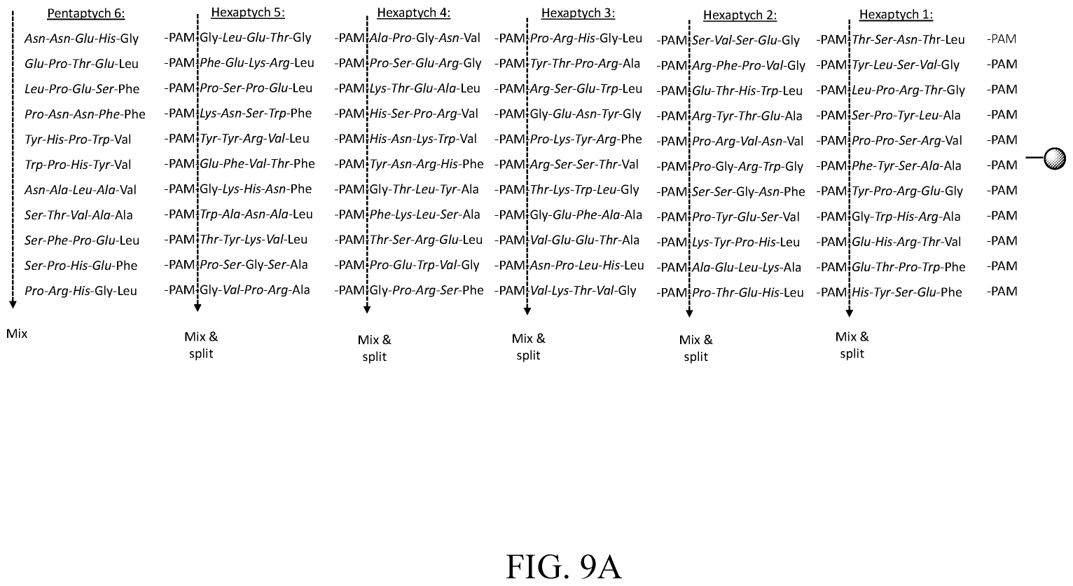

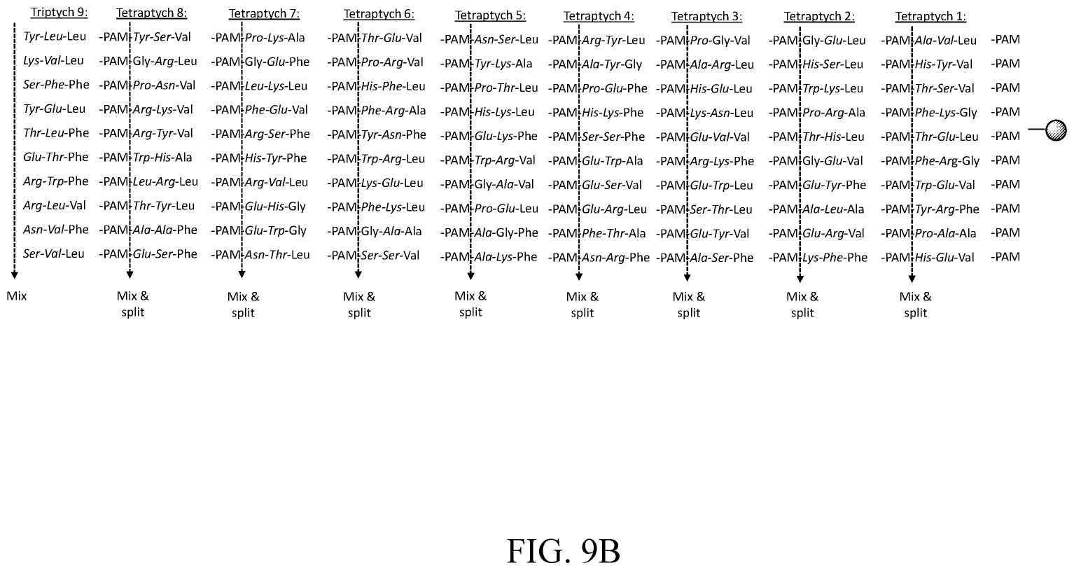

FIGS. 9A-9B illustrate examples of designed and validated libraries of synthetic compounds, in accordance with the present disclosure. Two large non-natural polymer libraries were synthesized, which are labeled as non-natural polymer (NNP)1 and NNP2.

FIG. 9A illustrates NNP1, which consists of six hexaptychs as the diversity elements in which each ptych were composed of four D-amino acids or glycine and an L-amino acid or glycine ester linked to a PAM linker. This produced polymers of 36 monomers in length with an average molecular weight of around 5 kDa. Each ptych was designed to have one of eleven possible hexaptychs per diversity position (listed under Hexaptych 1, Hexaptych 2, etc.), making a 11⁶ or an around 1.77 million compound library. This corresponds to 66 hexaptychs, each of which was designed through a selection of monomers to give a range of physicochemical properties in each sequence position and a unique molecular weight for each hexaptych. Before synthesizing the library, the synthetic feasibility of each individual hexaptych was confirmed as a reference to determine the retention time by LC-MS and facilitate the sequencing of hits (Table 4). Seventy-five copies of the library were made on 20-μm diameter monosized amino TentaGel™ microsphere resin beads with a loading of 0.27 mmol/g, which required around 550 mg of bead resin to produce.

FIG. 9B illustrates NNP2, which consists of nine tetraptychs constituting a total polymer length of 36 monomers. For each ptych in the sequence, there were 10 possible tetraptychs, constituting a total of 90 tetraptychs and creating a library of 109 or one billion compounds. This library was constructed on 10-μm beads and required 1.5 g of resin for production of three copies (total of three billion beads). The individual ptychs in this library were also synthesized as controls and validated for sequencing (SEE Table 5). After constructing the libraries, all side-chain protecting groups were removed and the libraries were screened against multiple biological targets.

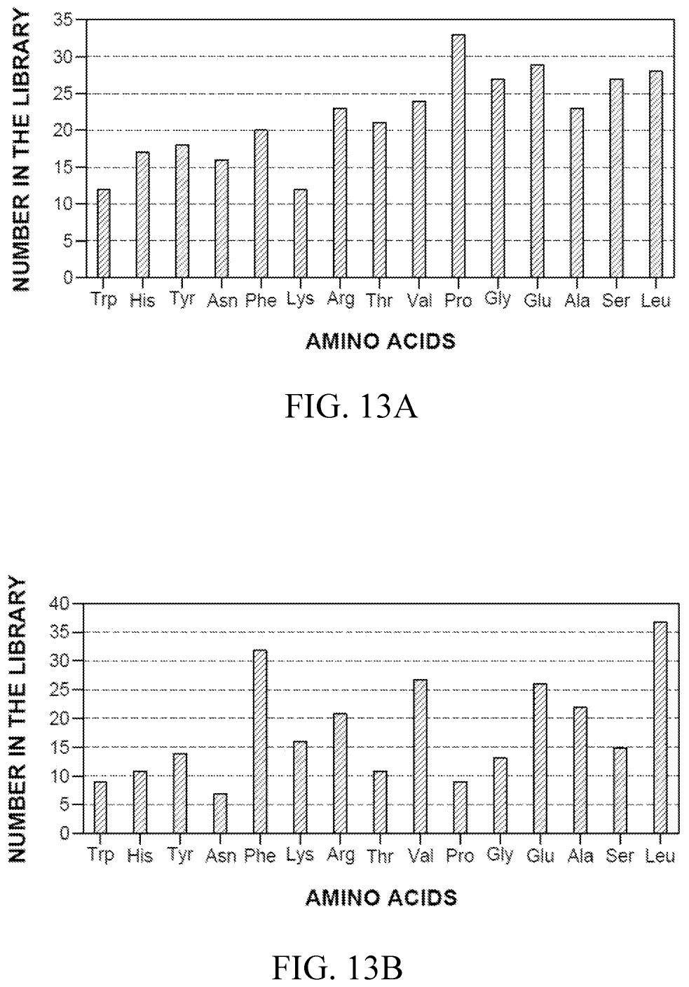

The two NNP libraries were constructed from two groups of building blocks. The first group included five pre-made fmoc-L-amino acid- (or Gly-) PAM esters: fmoc-L-Phe-PAM ester, fmoc-L-Ala-PAM ester, fmoc-L-Val-PAM ester, fmoc-L-Leu-PAM ester and fmoc-Gly-PAM ester. All the five amino acid-PAM-esters where commercially available with the boc-protecting group, which was converted to the fmoc form and was used in the library synthesis (see Supporting Information for synthesis). The second group of building blocks used in the library included 15 fmoc-protected D-amino acids (or Gly): Ala, Glu, Phe, Gly, His, Lys, Leu, Asn, Pro, Arg, Ser, Thr, Val, Trp and Tyr. NNP1 library was designed to have amino acid distribution closed to their average occurrence genome-wide (see FIG. 13A) (based on data from the UCSC Proteome Browser). The NNP1 library was designed in such a way that the amino acids were distributed among the six hexaptychs as shown in Table 15. The design of NNP2 library had less resemblance to the amino acid distribution in the genome (see FIG. 13B), and also here the amino acids were distributed among the nine tetraptychs in the library (see Table 16). Italics indicates D-amino acid. In the design, there were D-amino acids and PAM linker, which are both non-natural building blocks. L-amino acids were used at the position next to PAM as the corresponding fmoc-L-amino acid-PAMs were commercially available, whereas the D form was not.

To demonstrate the speed and efficiency of the screening and sequencing process the following five target protein were screened: K-Ras, ASGPR, IL-6, IL-6R, and TNFα. All are challenging targets for traditional small-molecule approaches and therefore represent interesting test cases for chemical NNP ligands.

FIGS. 10A-10D illustrate example results from a competitive binding assay, in accordance with the present disclosure. More particularly, FIGS. 10A-10D illustrate competitive binding for K-Ras, with the counter target being Raf. In the case of the K-Ras, IL6 and IL6R, a competitive binding assay was used to screen for PPI.

As shown by FIG. 10A-10B, for K-Ras, experimental embodiments screened for the blocking of binding of the Ras binding domain to the downstream signaling partner Raf. For IL-6, experimental embodiment screened for the blocking of binding to the receptor IL-6R and conversely in a separate screen to find binders of IL-6R that block IL-6 binding. The primary screening targets (e.g., Ras, IL-6, IL-6R) were labeled with dyes maximally excited at around 555 nm (e.g., AF555 or CF555) to identify binders in the FAST screen, and counter targets (e.g., Raf, IL-6R, IL-6) were labeled with dyes maximally excited at around 647 nm wavelength (e.g., AF647 or CF647) which can be detected by ADM on the CellCelector. After FAST screening hit detection the MFI for each dye on each bead was measured to prioritize the hits using both the overall brightness of the bead as a qualitative measure of binding affinity and the MFI ratio of the target to the counter target, as shown by FIGS. 10B-C. Only the single positive beads with fluorescence at 555 nm were picked and sequenced. This strategy was performed to enrich for inhibitors that would bind to K-Ras while blocking interaction with the downstream signaling partner Raf.

More particularly, FIG. 10A illustrates automated digital microscopic images demonstrating three types of hits. The top row includes single positive beads that bind the primary target K-Ras but not the counter target Raf. The middle row includes double single positive beads that bind both the primary target K-Ras and the counter target Raf. The bottom row includes single positive beads that bind the counter target Raf but not the primary target K-Ras. FIGS. 10B-10C illustrates hit deification by rapid FAST screen for K-Ras binding NNPs. FIG. 10B shows after FAST scan, the software filters out false positive hits including auto-fluorescence particles using the dual-wavelength comparison technology. Around 300 (e.g., 297) top hits with bright K-Ras-CF555 that are above the threshold were identified for further ADM/CellCelector (e.g., QA/QC) imaging and analysis from a 2.5 million 20-um bead sample plate. FIG. 10C shows the FAST hits identified in FIG. 10A were imaged, reviewed, and analyzed on CellCelector at high resolution. The MFI were measured for each true positive (TP) bead and the top ranked 55 TP hits based on high MFI of K-Ras-CF555 and ratio (CF555/Cy5) were isolated for sequencing, resynthesis and characterization. FIG. 10D are images representative of MST data showing the binding of resynthesized purified NNPs to the targets and the calculated K_{D} values.

In various experimental embodiments, K-Ras and ASGPR were screened against library NNP1. The library size of NNP1 is 1.77M members on 20-μm beads, and was screened at a 2.8-fold redundancy with 5M beads on two plates (2.5M beads per plate). Given the compound redundancy, hits were cross-validated via hit redundancy in the screen and used to find several hits with the same sequence (e.g., or 4-5 out of 6 hexaptychs in common, i.e., 67%-83% similarity within sequences). After FAST screening, a preliminary hit list of 381 K-Ras selective binding beads was identified. Hit sequences in the K-Ras screen were pooled into 14 clusters, and the most prevalent sequences in each case were selected from each cluster. Similarly, 190 hit sequences from the ASGPR screen were grouped into 19 clusters and individual hits from each cluster were selected for hit confirmation by resynthesis and measurement of K_{D} by MST (see Table 13). Equilibrium K_{D} binding affinities for K-Ras hits ranged from 18-180 nM, and 0.22-330 nM for ASGPR, as shown by FIG. 10D and Table 13.

With a library size of 1 billion members on 10-μm beads, library NNP2 requires 200 plates to screen the entire library at 5 M beads per plate. With a custom industrial robotic high-throughput screening (HTS) suite, this would be fairly straightforward—the entire library could be FAST screened in less than 10 hr. In this proof-of-concept study, a 10 million portion of the library was screened, corresponding to 2 screening plates against IL-6, IL-6R and TNFα. As with the K-Ras-Raf screen, the IL-6 screen was performed using counter labeled IL-6R labeled to identify IL-6 binding domain selective inhibitors. Similarly, the screen against IL-6R was performed using counter labeled IL-6. The hits were selected and isolated with the highest target to anti-target MFI ratios. For TNFa, a primarily interest was in finding selective affinity agents and the experiments did not conduct a competition screen. Binding affinities ranged from 25-500 nM for IL-6, 0.6-330 nM for IL-6R, and 0.3-270 nM for TNFα. The most potent hit in the NNP2 screen was a K_{D} of 620 pM against IL-6R.

Table 14 shows the hit rate broken down by screening and sequencing steps across the five targets. The average hit rate for beads identified by the FAST screen and passing QC/QA in ADM is 0.003% and ranged from 19 hit beads for IL-6 to 381 hits for K-Ras. The bead hit rate was determined by the threshold cut identified in assay development to eliminate the effects of auto-fluorescence producing false negatives. This is also a reasonable hit rate in terms of the downstream processing effort for hit confirmation. Hit bead selection for automated picking from the screening plates depends primarily on how isolated the beads are from neighboring beads. In some instances, hit beads were located in dense aggregates that made picking difficult to impossible without carrying over several other beads that will confound sequencing. In the five assays shown herein, on average allowed 72±44% of the hit beads to be picked. Of these on average 81±25% were successfully sequenced by LCMS. The factors affecting sequencing were successful transfer of the beads to the cleavage vial, where failure results in no observable ptychs, or incomplete sequencing where individual ptychs failed to be identified in the LCMS. Sequencing however did not prove to be a major challenge and the high sequencing rate enabled hit confirmation by resynthesis without major optimization. When a large number of redundant hits were identified, such as for K-Ras, ASGPR and IL-6R, sequences were clustered and the highest represented sequence in each cluster was selected for resynthesis. Over the five targets screened, hit confirmation of resynthesized hits was 71±29% denoting a high true positive rate.

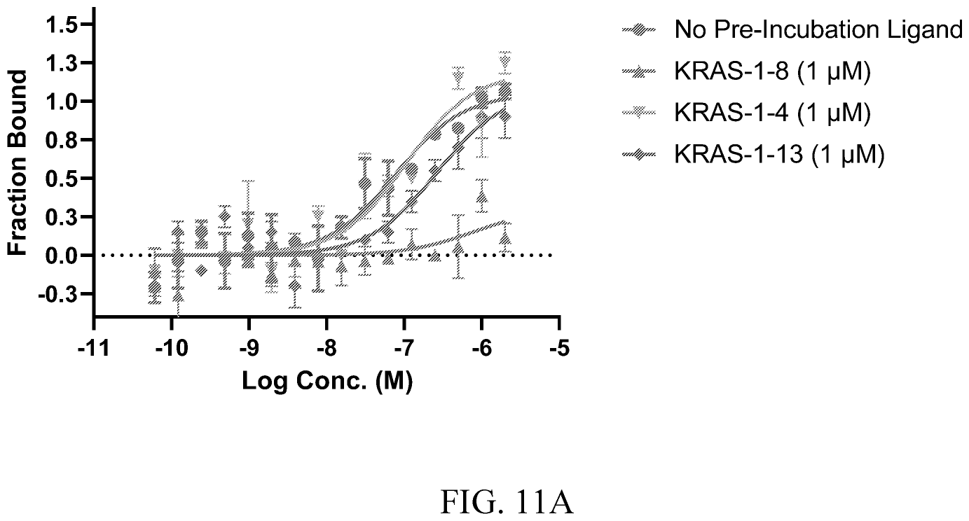

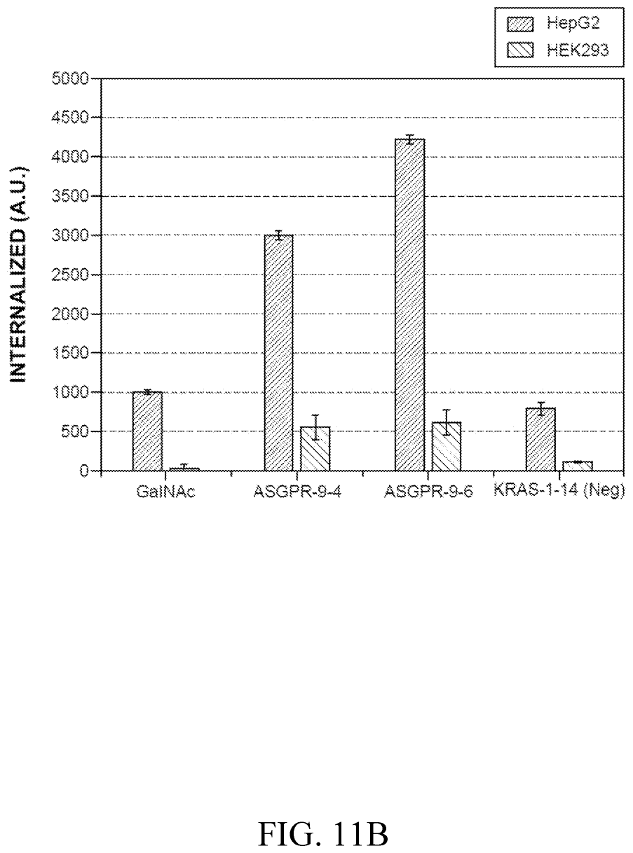

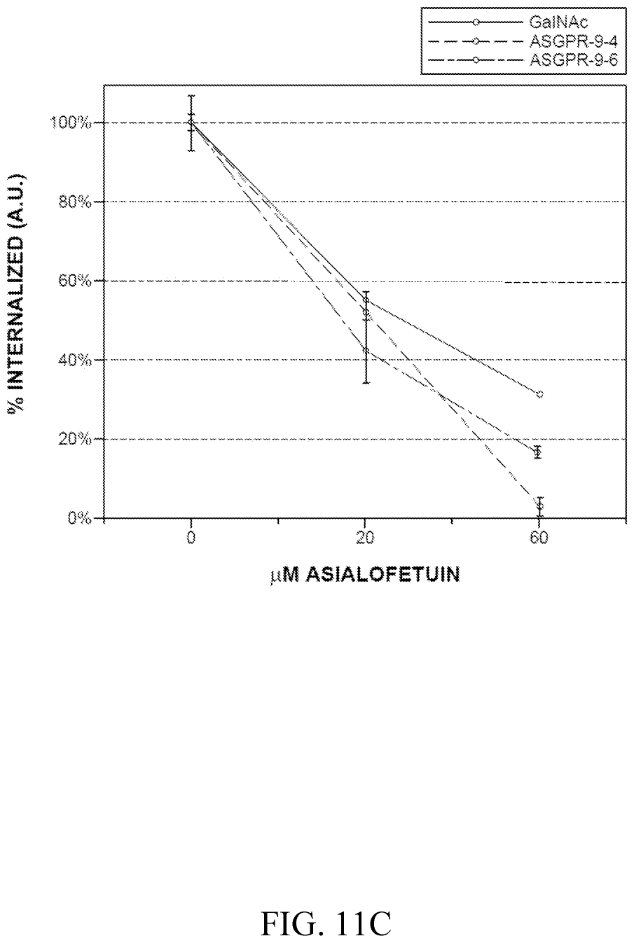

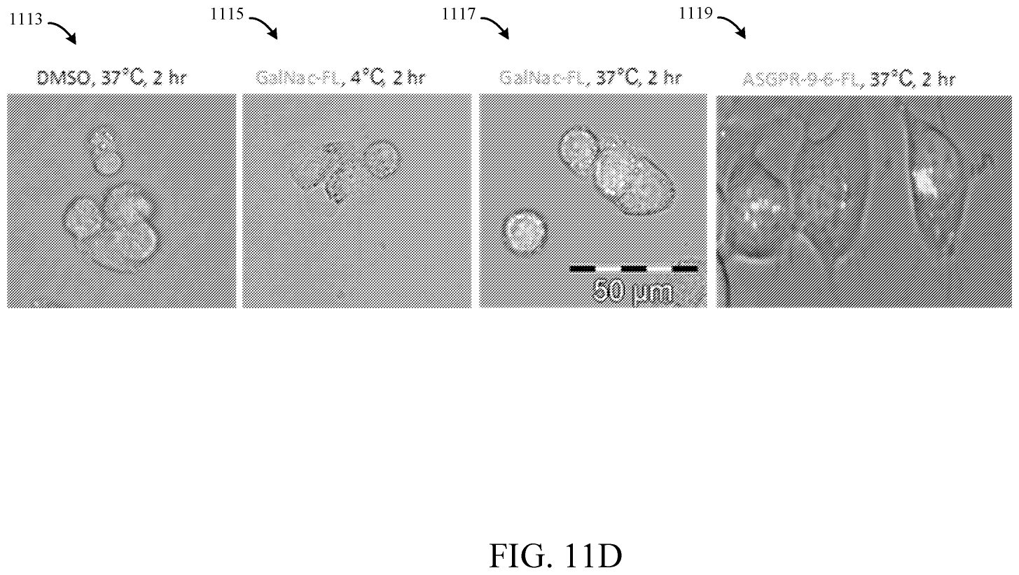

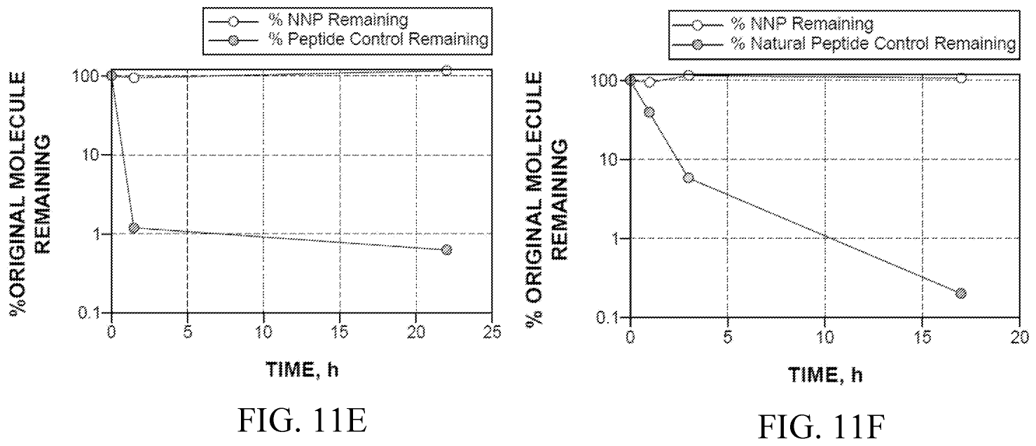

FIGS. 11A-11F illustrate example biological relevance and stability of NNPs in biological matrices, in accordance with the present disclosure.

FIG. 11A illustrates example results from measuring K-Ras-Raf interactions. To demonstrate the biological significance of NNPs and their target selectivity, the K-Ras and ASGPR functional biological activities were analyzed. For K-Ras, the specific inhibition of K-Ras and Raf binding for a range of confirmed K-Ras hits was measured using a fixed concentration of K-Ras (5 nM) and a titration of Raf to give a 78 nM binding affinity. This was tested on an MST competition binding assay (see FIG. 11A). As a control, the K-Ras-Raf interaction was measured alone for which an average K_{D} values of 78 nM was obtained from two technical runs. Then, the same interaction was measured with three different K-Ras lead hits (KRAS-1-4, K_{D}=36 nM; KRAS-1-8, K_{D}=44 nM; KRAS-1-13, K_{D}=30 nM; at 1 μM each), separately pre-incubated with K-Ras at room temperature for 15 minutes. The three NNPs showed a range of inhibition activity from complete inhibition (KRAS-1-8), partial inhibition (KRAS-1-13), and no inhibition (KRAS-1-4). The MFI ratios of the target (K-Ras-CF555) to the counter target (Raf-AF647) for the hit beads corresponding to these hits was higher for KRAS-1-8 than that of the other two hit-beads (MFI ratios: KRAS-1-8=2.55; KRAS-1-4=1.89 and KRAS-1-13=1.53), suggesting that this can be a useful metric for functional inhibitors of PPIs from the primary competition screen. More particularly, competitive inhibition of the K-Ras-Raf PPI was tested with a 15 minute pre-incubation with NNP ligands (1 pM concentration) followed by titration with Raf to measure binding by MST. NNP KRAS-1-8 showed complete inhibition, NNP KRAS-1-13 caused a shift in the K_{D} (K_{D}=260 nM), and NNP KRAS-1-4 showed no inhibition (K_{D}=100 nM).

FIGS. 11B-11D illustrate example results of measuring ASGPR and glycan interactions. ASGPR is a glycoprotein receptor, and previous published ligands are glycans that mimic the native substrates. To determine if NNP hits could specifically internalize into liver cells with high expression of ASGPR but not cells lacking ASGPR expression, the ASGPR-mediated uptake of two lead NNP hits were compared, ASGPR-9-4 (K_{D}=230 nM) and ASGPR-9-6 (K_{D}=34 nM), in the HepG2 human hepatocarcinoma (high expressing) and HEK293 (non-expressing) cell lines (FIG. 11B). As a positive control, the ASGPR trivalent ligand N-acetylgalactosamine (GalNAc) was used and a non-hit NNP from the same NNP1 library (KRAS-1-14) was used as a negative control. All compounds were labeled with fluorescein and analyzed by flow cytometry. Internalization of the two NNP hits was significantly higher in HepG2 cells compared to in HEK293 cells, and significantly higher than uptake of the positive control GalNAc. The non-hit NNP negative control showed minimal uptake in either cell line. To further demonstrate ASGPR mediated cellular uptake, competitive cell uptake assays were performed utilizing the two NNP hits and the positive control GalNAc in HepG2 cells in the presence of asialofetuin, a naturally occurring serum protein ligand for ASGPR (see FIG. 11C). Cells were pre-incubated with two concentrations of asialofetuin, (20 μM and 60 μM) representing 67 and 200-fold excess compared with the test compound (0.3 μM). The NNP hits and positive control's cellular uptake decreased with increasing concentrations of asialofetuin, and is mostly abolished with 60 μM of asialofetuin. These results indicate that these ligands compete for the same receptor, and that uptake is ASGPR-mediated. Importantly these results for K-Ras and ASGPR demonstrate that the NNP hits found in screening are not non-selective binders but are capable of selectively binding to the target proteins in the presence of other selective protein-binding partners, plasma media and cell membranes and are capable of eliciting functional biological responses.

More particularly, FIG. 11B illustrates cell uptake of GalNAc (positive control), two NNP hits (ASGPR-9-4 and ASGPR-9-6) and a non-hit NNP (KRAS-1-14, negative control) in HepG2 (high ASGPR expressing) verses HEK293 (low ASGPR expressing) cells lines. FIG. 11C illustrates competition uptake assay of GalNAc and two NNP hits (ASGPR-9-4 and ASGPR-9-6) in HepG2 cells after preincubation with different concentrations of asialofetuin (a naturally occurring serum protein ASGPR ligand). Decrease in cell uptake with increasing concentrations of asialofetuin indicates blocking of ASGPR-mediated uptake. FIG. 11D illustrates example confocal images 1113, 1115, 1117, 1119 of HepG2 cell uptake (scale: x20 for the images 1113, 1115, 1117 and x40 for the image 1119). FL=fluorescein label; Cell nucleus staining with DAPI (blue). No uptake with the blank sample (DMSO) is seen at 4 degrees C. Also, no uptake of the control GalNAc is seen at 4 degrees C. but is seen at 37 degrees C. Similarly intracellular uptake is observed for the NNP hit (ASGPR-9-6) at 37 degrees C. (but not at 4 degrees C.—not shown).

FIGS. 11E-11F illustrate example results for measuring synthetic compounds targeted to IL-6R. To confirm the superiority in stability of these largely D-amino acid NNPs over peptides, the stability of an IL-6R hit from NNP2 to proteinase K and in human plasma was investigated. For the proteinase K stability assay, the original hit was compared with its fully L-amino acid variant. The L-amino acid variant was completely degraded within less than two hours in the presence of proteinase K (see FIG. 11E), whereas minimal degradation was observed for the NNP hit after an overnight incubation. Similar stability was observed in human plasma, where the stability of the hit was compared to the natural peptide Angiotensin I. Angiotensin I was completely degraded within four hours in human plasma (see FIG. 11F), whereas NNP2 hits stayed largely intact even after overnight incubation. More particularly, FIG. 11E illustrates stability data for NNPs screened against IL6R-87-8 compared to its L variant in the presence of proteinase K and FIG. 11F illustrates data for the same NNP in human plasma compared with Angiotensin I.

Using the FAST screening platform and ptych design, experimental embodiments have demonstrated a mega-throughput screening and sequencing strategy for the discovery of potent and functional NNPs. The ability to screen at the femtomole scale on 10-μm beads enables time and cost-effective screening with much larger chemical diversity than has previously been reported. In some experimental embodiments, commercially available amino acid building blocks and solid phase chemistry was used to construct the NNP libraries to validate the screening and sequencing methodology. Using the same approach, synthetic building blocks and coupling chemistry as well as different cleavable linkers can be used enabling an unlimited access to polymer diversity through library synthesis and empirical screening. Experimental embodiment further demonstrated finding low nanomolar to picomolar hits from primary screening and have used this to validate biological selectivity and activity in a range of biological or molecular targets. Further shown is the identification of biological functionality of the hits of two targets as representative use cases, which are the ability to disrupt PPI by inhibition of the K-Ras/Raf interaction, and protein-glycan interaction (PGI) in ASGPR-mediated cellular uptake and internalization. Utilizing a similar approach, experimental embodiments have additionally shown NNPs that can used as be intracellular delivery agents by targeting ASGPR to identify receptor selective NPPs that not only bind ASGPR but are actively transported across cell membranes in a selective receptor mediated manner. The NNP hits identified, without optimization, are more efficiently intracellularly transported than the previously reported molecular transport ligand trivalent GalNAc which is being used commercially for the delivery of nucleic acid drugs. This is particularly noteworthy as GalNAc and other reported ligands for ASGPR are glycans and experiments have demonstrated that ASGPR can also bind and transport non-natural peptide-like ligands. Lastly, the primarily D-amino acid NNPs show unique stability against biological degradation.

Transition melt temperatures across all hits ranged from 39-65 degrees C. (data not shown), which is within the range of folded proteins of similar lengths and indicates that tertiary structure is probably important for the molecular interactions of these hits. As the diversity of synthetic polymers expands, 3D structures of hits by crystallography or nuclear magnetic resonance (NMR) may identify templates for novel secondary and tertiary structural motifs that can be rapidly refined by building focused libraries for secondary screening. As structural motifs become better understood, individual ptychs can be engineered to promote intra- and inter-molecular recognition to stabilize structure and maximize affinity. In various experiments, regular repeating ptych designed for libraries described herein were used, but more elaborate designs that use different numbers and types of monomers in ptych positions are possible. These will provide further chemical diversity for primary screening and strategies that allow optimization of hits. Such experiments identified designer polymers with completely new structures and functions through empirical screening. The application area of this platform is vastly broad and includes therapeutics for drug discovery, affinity reagents for sensors and diagnostics, and reagents for catalysis.

The following describes various different specific methods conducted for the above described experiments.

Synthesis of Libraries NNP1 and NNP2.

All libraries were synthesized using “one-bead-one-compound” and “mix-and-split” methods of solid-phase synthesis on TentaGel™ amine 10 m or 20 μm resin. Library NNP1 was synthesized on 554 mg 20 μm TentaGel™ M NH₂ (0.27 mmol/g amine loading) with theoretical diversity of 1.77×10⁶ and 75 copies (i.e., 1.33×10⁸ beads). Library NNP2 was synthesized on 1.5 g 10 μm TentaGel™ M NH₂ (0.25 mmol/g amine loading) with theoretical diversity of 1×10¹ and 3 copies (i.e., 3×10¹ beads).

For the synthesis of library NNP1, the beads were swollen in DCM for 1 hour. Then the DCM was drained, and the beads were suspended in DMF and were divided evenly by pipet between 11 plastic fritted syringes placed on a manifold. Then 11 different hexaptychs were constructed on the beads, a different hexaptych in each fritted syringe, by coupling first an L-amino acid-PAM ester followed by the coupling of four more D-amino acids, according to the library design in FIG. 9A. The beads were then mixed and split evenly again between the 11 plastic fritted syringes, and the synthesis was carried on in the same manner with the next hexaptychs, until all the six hexaptychs were constructed.

For the synthesis of library NNP2, after swelling the beads in DCM for 1 hour, the DCM was drained, and the beads were suspended in DMF. Then, the beads were divided evenly by pipet between 10 plastic fritted syringes placed on a manifold. Then 10 different tetraptychs were constructed on the beads, a different tetraptych in each fritted syringe, by coupling first an L-amino acid-PAM ester followed by the coupling of two more D-amino acids, according to the library design in FIG. 9B. The beads were then mixed and split evenly again between the 10 plastic fritted syringes, and the synthesis was carried on in the same manner with the next tetraptychs, until all the nine tetraptychs were constructed.

Coupling Conditions for Fmoc-L-Amino Acid-PAM Esters in the Library Synthesis:

3.5 eq. fmoc-L-amino acid-PAM ester was dissolved in a solution of 0.5 M HATU in NMP (3.18 eq. HATU). Then DIEA (10 eq.) was added to this mixture to activate the amino acid for 30 seconds, and the solution was added to the resin and reacted for 30 minutes. After completion of the coupling reaction (confirmed by ninhydrin test), the resin was drained and washed with DMF (3×5 mL).

Coupling Conditions for Fmoc-D-Amino Acids:

5.5 eq. fmoc-D-amino acid was dissolved in a solution of 0.5 M HATU in NMP (5 eq. HATU). Then DIEA (10 eq.) was added to this mixture to activate the amino acid for 30 seconds, and the solution was added to the resin and reacted for 30 minutes. After completion of the coupling reaction (confirmed by ninhydrin test), the resin was drained and washed with DMF (3×5 mL).

Fmoc Deprotection:

Fmoc deprotection was performed by the addition of 25% 4-methyl-piperidine in DMF (5 mL) to the resin (1×5 min+1×10 min), followed by draining and washing the resin with DMF (5×5 mL).

Side-Chain Deprotection:

At the end of the construction of the library, after the last fmoc deprotection, all the library beads were mixed into one fritted syringe and the side-chain protecting groups were removed with a solution of 95% (v/v) TFA, 2.5% (v/v) water and 2.5% (v/v) triisopropylsilane (1 mL of cleavage solution per 10 mg of resin) for 2 hours. Then the TFA cocktail was drained, and the resin was thoroughly washed with DCM, DMF, DCM and MeOH (3×10 mL of each solvent) and was ready for the screening process.

Activation of K-Ras by GTP Loading for the Screen and Binding Assays.

To activate K-Ras for binding NNP or Raf, the K-Ras protein had to be loaded with GTP. Loading was performed according to the following protocol: The 200 μM stock solution of the target protein was diluted to 10 μM in 20 mM HEPES pH 8.0, 150 mM NaCl, 10 mM MgCl₂, 1 mM TCEP, 0.05% Tween-20 (total volume 110 μL). 10 μL were set aside for later labeling quality control. EDTA pH 8.0 (stock concentration 10 mM) was added to the protein solution to a final concentration of 80 μM. GTP (stock concentration 50 mM) was added to the protein solution to a final concentration of 750 μM. The solution was incubated at 30° C. for 2 hours (PCR tube) and then placed on ice for 2 minutes. MgCl₂ was added to the protein solution to a final concentration of 100 mM. The resulting protein solution was buffer exchanged into the buffer required in the labeling kit for labeling. This procedure was used before the screen, the MST analysis and the K-Ras/Raf inhibition assay. All the other target molecules (TNFα, IL-6, IL-6R and ASGPR) were used as received without any additional treatment.

Screening of OBOC Libraries on FAST.

FAST screening assay were specifically optimized for each target in terms of probe concentration, blocking and washing stringency etc. The probe binding to the NNP1 and NNP2 library beads was performed in tubes. Typically, the library or control beads were hydrated in the buffer (1% PEG, 50 mM Tris, pH 7.5, 25% Odyssey blocking buffer PBS) for 30 min at RT with vortex followed by 1 min of sonication to break apart the large bead clumps. Beads were then centrifuged down, and the bead pallets were washed 2× with Odyssey/PBS buffer, the bead suspension was further filtered through a 30-um size cell strainer to remove bead aggregates. The concentration of the hydrated beads was determined based on bead counting using a hemocytometer. Aliquots of the bead suspension with the required number of beads then were centrifuged down and resuspended in blocking buffer (100% Odyssey, 0.5% Chaps, 200 mM NaCl in PBS) and incubated overnight at RT with gentle rotating. After blocking, the beads were pelleted and resuspended in 100% Odyssey buffer and then mixed at a 1:1 volume ratio) with the CF555 or AF555 conjugated probe that was diluted in the pre-binding buffer (1% Chaps, 400 mM NaCl, 2 mM TCEP, in PBS) to 2× final working concentration. The probe/library bead mixture were incubated for 1 hour at room temperature with gentle rotation to allow probe bind to the library beads. After incubation, the beads were palleted and the unbound probes were aspirated, followed by 3 washes with 10 ml of wash buffer (0.5% Chaps, 200 mM NaCl, 1 mM TCEP in PBS), 5 minutes/time and additional 2 washes with 10 ml of 0.5% Chaps/PBS. After the last centrifugation, the buffer was aspirated but left final around 500 μl to resuspend the beads in this residual buffer and sonicate them for 30 second to dissociated newly formed bead clumps. Then 1.5 ml prepared 0.3% low melting agarose (LMT) that were kept in 37 degree C. water-bath before use were added to into the re-sonicated beads to make the bead/soft agar suspension.

Beads in the LMT suspension were then transferred and evenly plated onto the FAST slide (the screening plates) and then the slides were placed on cold tray to accelerate the curing and immobilization of the beads. Following the gel formation, a layer of mounting medium (e.g., 500 μl Live-Cell medium) was gently placed on top of the gel to keep the beads from rapid drying or photo-quenching of the fluorescence. The sample slides (plates) were scanned and analyzed using the FAST system. The FAST analysis generates a bead hit list, where each bead is quantified by a MFI measurement.

Bead Analysis and Picking Using ALS CellCelector.

The beads with MFI values above a threshold determined by the “no probe” control condition were identified and then coordinate list of the hits were transferred to the CellCelector for ADM. This imaging analyzes the hits with multiple channels at higher resolution. Images of the hit beads were then QC/QA reviewed based on the morphology and fluorescence staining, and fluorescence of selected channels were quantified to rank the top hits for isolation, then each selected single hit-beads was isolated with CellCelector individually into the HPLC vials in ddH₂O for MS based sequencing.

Processing and Sequence Analysis of Picked Beads.

Beads were deposited directly into glass autosampler vials containing deionized water. The vials were inserted into deep-well 96-well plates and dried in a vacuum centrifugal concentrator (GeneVac II Plus) at 40 degrees C. To hydrolyze the inter-ptych ester linkages 50 μL of 7% aqueous ammonium hydroxide or 150 mM NaOH was added, and the samples incubated at 37 degrees C. for 6 hours and then evaporated under vacuum in the centrifugal concentrator. The samples were then prepared for analysis by adding 50 μL of 5% acetonitrile in water with 0.1% formic acid and analyzed by capillary reversed-phase gradient LC-MS/MS using an Agilent capillary HPLC pump and CTC Analytics autosampler coupled to an LTQ-Orbitrap mass spectrometry system. Expected masses of hydrolysis products were loaded into an inclusion list for targeted MS/MS when detected above threshold in a high resolution Orbitrap scan. Data analysis used both MS and MS/MS data to assign high confidence hits for assembling sequences for the hits.

Hit Re-Synthesis.

Solid Phase NNP Synthesis:

Hits were synthesized on ChemMatrix Rink amide resin (loading 0.5 mmol/g, typical scale: 30 mg, 0.015 mmol) by an automated peptide synthesizer (Biotage Syro I) using standard fmoc-based amide coupling conditions with DIC/Oxyma as the coupling reagents. Fmoc protected L-amino acid-PAM esters used in the library synthesis were replaced here by two separate residues: fmoc-L-amino acid and fmoc-PAM. This was in order to avoid having ester linkage (but rather a standard amide linkage) in the synthesized hits, for stability purposes. Synthesis was performed using the following protocol: ChemMatrix Rink amide resin was swollen in DCM for 1 hour, drained, washed with DMF and placed on the peptide synthesizer for constructing the full sequence. Fmoc deprotection was performed by the addition of 25% 4-methyl-piperidine in DMF (1.2 mL) to the resin (1×5 minutes+1×10 minutes), followed by draining and washing the resin with DMF (5×1.2 mL). Couplings were performed by adding 250 μL of NMP to the resin followed by 90 μL of 0.5 M fmoc-protected amino acids (or fmoc-PAM) in DMF (3 eq., 0.045 mmol), 90 μL of 0.5 M Oxyma in DMF (3 eq., 0.045 mmol) and 90 μL of 0.5 M DIC in DMF (3 eq., 0.045 mmol). The resin-mixture was allowed to react for 15 minutes at 60 degrees C. and was then drained, washed with DMF (3×1.2 mL) and treated again with the same coupling conditions for double coupling. At the end of the double coupling, the fmoc was deprotected and these synthesis cycles were repeated on the peptide synthesizer until all the residues were constructed onto the resin. After the last fmoc-deprotection, the resin-NNP was taken out of the peptide synthesizer for manual fluorescein incorporation.

Incorporation of Fluorescein:

fluorescein was incorporated on the N-terminus of all the re-synthesized hits. 21.3 mg NHS-fluorescein (3 eq., 0.045 mmol) was dissolved in 300 μL DMF and was added to the resin-NNP. The resin-mixture was allowed to react for 3 hours and was monitored by ninhydrin test. Upon completion, the resin was drained and washed thoroughly with DMF (3×5 mL) and DCM (3×5 mL) and was dried before cleavage.

NNP Cleavage:

Cleavage from solid support and side-chain deprotection were performed by treatment of resin-NNP with a solution of 95% (v/v) TFA, 2.5% (v/v) water and 2.5% (v/v) triisopropylsilane (3 mL of cleavage solution per 30 mg of resin) for 2 hours. TFA was then evaporated on the SpeedVac (Thermo Scientific Savant SpeedVac Concentrator) until the solution volume reached to around 1 mL. The crude NNP was then precipitated and triturated with chilled diethyl ether (×3) and was then purified by preparative LC-MS as described above.

Hit Characterization.

Hit binding affinity to various targets were determined using Microscale Thermophoresis (MST). MST experiments were performed on a Monolith NT.115pico (NanoTemper Technologies GmbH, Munich, Germany). Measurements were performed at room temperature, in triplicate with incubation periods of 15, 30 and 45 minutes. Binding affinities were obtained from a 16 point, two fold dilutions series with ligand starting concentration at 1 μM and target concentration at 5 nM. Targets were labeled using Nanotemper Monolith 2^{nd} Generation Protein Labeling Kits. RED-MALEIMIDE (Maleimide-647-dye) labeling kit was used for K-Ras and RED-NHS (NHS-647-dye) labeling kit was used for IL-6, IL-6R, TNFα and ASGPR. The buffer for the ASGPR contained 20 mM HEPES pH 7.4, 150 mM NaCl, 10 mM MgCl₂, 2 mM CaCl₂, 0.05% Pluronic F-127, and 1 mM DTT; for IL-6 contained 20 mM HEPES pH 7.4, 150 mM KCl, 10 mM MgCl₂, and 0.1% Pluronic F-127; for the soluble IL-6 receptor 20 mM HEPES pH 7.4, 150 mM NaCl, 10 mM MgCl₂, and 0.05% Tween-20; for K-Ras 20 mM HEPES pH 7.4, 150 mM NaCl, 10 mM MgCl₂, 0.05% Tween-20 and 1 mM DTT; for TNFα 10 mM HEPES pH 7.4, 150 mM NaCl, 10 mM MgCl₂, 0.05% Polysorbate-20. Triplicate data was analyzed using MO.AffinityAnalysis software (NanoTemper Technologies GmbH).

K-Ras/Raf Inhibition Assay.

Interaction between the target protein K-Ras and the ligand protein Raf was investigated using a MicroScale Thermophoresis (HTS-MST) assay in the absence and presence of three NNP hits: KRAS-1-4, KRAS-1-8 and KRAS-1-13. Loading of K-Ras with the GTP was performed according to the protocol detailed above (Activation of K-Ras by GTP loading). After the GTP loading the resulting protein solution was buffer exchanged into 100 mM HEPES pH 6.5, 5 mM MgCl2, 50 mM NaCl, 1 mM TCEP. The resulting concentration of the target protein was 8.9 μM, which was used for Maleimide-647-dye labeling. For the interaction between K-Ras and Raf with no NNP present two technical runs with the same samples were performed between GTP-loaded K-Ras and Raf in the same buffer conditions that were used to test the interaction between K-Ras and the NNP hits: 20 mM HEPES pH 7.4, 150 mM NaCl, 10 mM MgCl2, 1 mM DTT, 0.05% Tween-20. For the interaction between K-Ras and Raf in the presence of NNPs the labeled target protein K-Ras was diluted to 10 nM in assay buffer containing 2 μM NNP and incubated at room temperature for 15 minutes. This solution was then mixed with the ligand protein Raf serial dilution 1:1 to yield the final assay samples with 5 nM target protein and 1 μM NNP.

Cell Culture for ASGPR Uptake Assay.

The HEK293T (human embryonic kidney cells) and human hepatoma HepG2 cells were grown according to the protocols provided by the American Type Culture Collection (ATCC). When cells reached cell were seeded at ˜1.5×10⁵ cells/well in 24-well culture plate for the uptake assay. After at least 16 hours of culture to allow cell attach and equilibrate, the compound treatment was set up for the uptake assay.

ASGRPR Uptake Assay.

The fluorescein-labeled NNPs or fluorescein-labeled N-acetylgalactosamine (GalNAc) recognizing ASGRPR were added to wells at indicated concentrations and incubated for 2 hours. Two plates were prepared for each treatment condition, one serving as 4 degrees C. no internalization control that was kept on ice during incubation, while the second plate was incubated at 37 degrees C. to allow for energy dependent internalization. Following the incubation period, all plates were placed on ice and washed three times with ice-cold PBS/3% BSA/2 mM EDTA and then lifted with trypsin. Cells were transferred to a 96-well round bottom plates in FACS buffer. Cells were then analyzed by flow cytometry using LSR-HJ with HTS sampler (BD Biosciences, San Jose, CA, USA). Data (mean fluorescence intensities) and further analyzed using Flowjo software (BD Biosciences, San Jose, CA, USA). The internalized fraction was expressed as the difference between the corresponding 4 degrees C. and 37 degrees C. MFIs as previously described.

For the competitive uptake assay with the natural ligand of ASGPR (asialofetuin), cells were pre-incubated with 20 μM and 60 μM asialofetuin on ice or at 37 degrees C. for 1.5 hours, followed by the treatment with compounds for additional 2 hours before flow cytometric analysis as described above. The reduction of the uptake under asialofetuin competition was expressed by the percentage against the same treatment condition without asialofetuin.

Stability Assays.

For proteinase K stability, solutions of each tested compound (200 μM) in 10% DMSO and 20 mM Tris HCl at pH 8 were prepared. Proteinase K was added to a final concentration of 100 pg/mL and 100 μM of the tested compound in 5% DMSO and 10 mM Tris HCl at pH 8. The solutions were incubated at 37 degrees C. and aliquots after 0 hours, 1.5 hours and 16 hours were analyzed by LC-MS.

For human plasma stability, lyophilized human plasma was reconstituted in sterile water for injection, aliquoted into 200 uL aliquots and stored frozen at −80 degrees C. prior to the stability studies. For the stability studies three aliquots per NNP were thawed at room temperature. An additional set of three aliquots for a positive control peptide (Angiotensin I) were also thawed. The incubations for the plasma stability were initiated by mixing 2 uL of a 2 mM stock solution of NNP IL6R-87-8 or positive control in DMSO with the 200 uL thawed plasma aliquot. After briefly vortex-mixing, 50 uL zero-time-point samples were removed, mixed with 50 uL of water and 400 uL of acetonitrile and frozen on dry ice until all time point samples had been collected. The samples were incubated at 37 degrees C. with samples removed and water/acetonitrile added at 1 hour, 3 hours, and 17 hours. Proteins were precipitated by centrifuging the samples at 17,000 G, 4 degrees C. for 1 hour. The supernatants were removed and concentrated in a centrifugal vacuum concentrator (GeneVac Genie II) at 45 degrees C. until the volume had been reduced to around 60 uL. The samples were then diluted with 95% water 5% Acetonitrile and 0.1% Formic Acid to volume of 200 uL, 2 uL of an internal standard peptide (Val5-Angiotensin I, Sigma) were added prior to samples analysis by LC-MS on the LTQ-Orbitrap XL system described above.

Table 1 below illustrates the effects of bead size on total amount of material, resin, and monomer requirements for one-bead one-compound (OBOC) library synthesis.

TABLE 1

| | Loading per bead | | Total library resin needed | |
|---|---|---|---|---|
| TentaGel ™ | (amine loading of 0.2 | | for 1B-sized library with | |
| bead size | mmol/g) | | one copy | |
| 10 μm | 100 | fmol | 513 | mg |
| 30 μm | 4 | pmol | 22 | g |
| 90 μm | 0.1 | nmol | 350 | g |

Table 2 illustrates the detection/recovery rate of FASTact platform by “spiking” studies (a small number of positive beads were diluted in the background “naked” TentaGel beads). Abbreviation: Streptavidin-AlexaFluor 555 (SA-AF555). Detection rates above 100% were observed at the lowest dilutions indicating a small amount of false positives.

TABLE 2

| | Positive | Background | | | Detection |
|---|---|---|---|---|---|
| | beads | beads | Probe | Dilution ratio | rate |
| 1 | 20-μm | 20-μm naked | 50-nM | 100, 500, or | 95-121% |
| | biotin- | TentaGel | SA-AF555 | 1000:10 | |
| | beads | beads | | million | |
| | | | | (1:100,000 | |
| | | | | 1:20,000 | |
| | | | | 1:10,000) | |

FIG. 12 illustrates an example detection rate, in accordance with the present disclosure. More particularly, FIG. 12 is a graph showing the example detection rate of FAST positive beads spiked in the background beads. A total of 100, 500, or 1000 biotin conjugated beads were spiked into 10×10⁶ unmodified (“naked”) beads at concentrations of 1:10,000, 1:2,000, and 1:1,000 respectively and stained with SA-AF555. The samples were scanned by FAST and the hits were identified; then, positive beads were confirmed by ADM imaging as described in the methods. The average detection rate from three experiments was above 95%.

Tables 3A-3D illustrate different repeated sequencing of single beads of a known sequence. The sequences are presented using single-letter amino acid codes; only those on the N-terminal side of the PAM-O-Ester (O) are the natural L-configuration, and the remaining sites contain the corresponding unnatural D-stereoisomers.

Table 3A shows sequences obtained from 24 individual beads synthesized with the same hit sequence from the NNP1 library. No false positive hits were observed for any of the ptychs, but five of the bead samples generated no sequence.

TABLE 3A

| Sample-Vial | Sequence Read |
|---|---|
| 16665-94-1-A1 | LPESF_O_GVPRA_O_GPRSF_O_YTPRA_O_RFPVG_O_LPRTG |
| 16665-94-1-A2 | LPESF_O_GVPRA_O_GPRSF_O_YTPRA_O_RFPVG_O_LPRTG |
| 16665-94-1-A3 | LPESF_O_GVPRA_O_GPRSF_O_YTPRA_O_RFPVG_O_LPRTG |
| 16665-94-1-A4 | LPESF_O_GVPRA_O_GPRSF_O_YTPRA_O_RFPVG_O_LPRTG |
| 16665-94-1-A5 | LPESF_O_GVPRA_O_GPRSF_O_YTPRA_O_RFPVG_O_LPRTG |
| 16665-94-1-A6 | LPESF_O_GVPRA_O_GPRSF_O_YTPRA_O_RFPVG_O_LPRTG |
| 16665-94-1-A7 | LPESF_O_GVPRA_O_GPRSF_O_YTPRA_O_RFPVG_O_LPRTG |
| 16665-94-1-B1 | LPESF_O_GVPRA_O_GPRSF_O_YTPRA_O_RFPVG_O_LPRTG |
| 16665-94-1-B2 | LPESF_O_GVPRA_O_GPRSF_O_YTPRA_O_RFPVG_O_LPRTG |
| 16665-94-1-B3 | LPESF_O_GVPRA_O_GPRSF_O_YTPRA_O_RFPVG_O_LPRTG |
| 16665-94-1-B4 | LPESF_O_GVPRA_O_GPRSF_O_YTPRA_O_RFPVG_O_LPRTG |
| 16665-94-1-B5 | LPESF_O_GVPRA_O_GPRSF_O_YTPRA_O_RFPVG_O_LPRTG |
| 16665-94-1-B6 | LPESF_O_GVPRA_O_GPRSF_O_YTPRA_O_RFPVG_O_LPRTG |
| 16665-94-1-B7 | LPESF_O_GVPRA_O_GPRSF_O_YTPRA_O_RFPVG_O_LPRTG |
| 16665-94-1-B8 | LPESF_O_GVPRA_O_GPRSF_O_YTPRA_O_RFPVG_O_LPRTG |
| 16665-94-1-B9 | LPESF_O_GVPRA_O_GPRSF_O_YTPRA_O_RFPVG_O_LPRTG |
| 16665-94-1-B10 | LPESF_O_GVPRA_O_GPRSF_O_YTPRA_O_RFPVG_O_LPRTG |
| 16665-94-1-B11 | LPESF_O_GVPRA_O_GPRSF_O_YTPRA_O_RFPVG_O_LPRTG |
| 16665-94-1-B12 | LPESF_O_GVPRA_O_GPRSF_O_YTPRA_O_RFPVG_O_LPRTG |

Table 3B shows sequences obtained from twenty-two individual beads synthesized with the same hit sequence from the NNP1 library. No false positive hits were observed for any of the ptychs, but two of the bead samples generated no sequence.

TABLE 3B

| Sample-Vial | Sequence Read |
|---|---|
| 16665-94-2-A1 | LPESF_O_FEKRL_O_YNRHF_O_RSEWL_O_RFPVG_O_LPRTG |
| 16665-94-2-A2 | LPESF_O_FEKRL_O_YNRHF_O_RSEWL_O_RFPVG_O_LPRTG |
| 16665-94-2-A3 | LPESF_O_FEKRL_O_YNRHF_O_RSEWL_O_RFPVG_O_LPRTG |
| 16665-94-2-A4 | LPESF_O_FEKRL_O_YNRHF_O_RSEWL_O_RFPVG_O_LPRTG |
| 16665-94-2-A5 | LPESF_O_FEKRL_O_YNRHF_O_RSEWL_O_RFPVG_O_LPRTG |
| 16665-94-2-A6 | LPESF_O_FEKRL_O_YNRHF_O_RSEWL_O_RFPVG_O_LPRTG |
| 16665-94-2-A8 | LPESF_O_FEKRL_O_YNRHF_O_RSEWL_O_RFPVG_O_LPRTG |
| 16665-94-2-A9 | LPESF_O_FEKRL_O_YNRHF_O_RSEWL_O_RFPVG_O_LPRTG |
| 16665-94-2-A11 | LPESF_O_FEKRL_O_YNRHF_O_RSEWL_O_RFPVG_O_LPRTG |
| 16665-94-2-A12 | LPESF_O_FEKRL_O_YNRHF_O_RSEWL_O_RFPVG_O_LPRTG |
| 16665-94-2-B1 | LPESF_O_FEKRL_O_YNRHF_O_RSEWL_O_RFPVG_O_LPRTG |
| 16665-94-2-B2 | LPESF_O_FEKRL_O_YNRHF_O_RSEWL_O_RFPVG_O_LPRTG |
| 16665-94-2-B3 | LPESF_O_FEKRL_O_YNRHF_O_RSEWL_O_RFPVG_O_LPRTG |
| 16665-94-2-B4 | LPESF_O_FEKRL_O_YNRHF_O_RSEWL_O_RFPVG_O_LPRTG |
| 16665-94-2-B5 | LPESF_O_FEKRL_O_YNRHF_O_RSEWL_O_RFPVG_O_LPRTG |
| 16665-94-2-B6 | LPESF_O_FEKRL_O_YNRHF_O_RSEWL_O_RFPVG_O_LPRTG |
| 16665-94-2-B7 | LPESF_O_FEKRL_O_YNRHF_O_RSEWL_O_RFPVG_O_LPRTG |
| 16665-94-2-B8 | LPESF_O_FEKRL_O_YNRHF_O_RSEWL_O_RFPVG_O_LPRTG |
| 16665-94-2-B9 | LPESF_O_FEKRL_O_YNRHF_O_RSEWL_O_RFPVG_O_LPRTG |
| 16665-94-2-B10 | LPESF_O_FEKRL_O_YNRHF_O_RSEWL_O_RFPVG_O_LPRTG |

Table 3C shows sequences obtained from twenty-two individual beads synthesized with the same hit sequence from the NNP2 library. No false positive hits were observed for any of the ptychs, and the full sequence was obtained on every bead sample.

TABLE 3C

| Sample-Vial | Sequence Read |
|---|---|
| 16665-94-3-D1 | NVF_O_YSV_O_RSV_O_FRA_O_YKA_O_ERL_O_ARL_O_PRA_O_FKG |
| 16665-94-3-D2 | NVF_O_YSV_O_RSV_O_FRA_O_YKA_O_ERL_O_ARL_O_PRA_O_FKG |
| 16665-94-3-D3 | NVF_O_YSV_O_RSV_O_FRA_O_YKA_O_ERL_O_ARL_O_PRA_O_FKG |
| 16665-94-3-D4 | NVF_O_YSV_O_RSV_O_FRA_O_YKA_O_ERL_O_ARL_O_PRA_O_FKG |
| 16665-94-3-D5 | NVF_O_YSV_O_RSV_O_FRA_O_YKA_O_ERL_O_ARL_O_PRA_O_FKG |
| 16665-94-3-D6 | NVF_O_YSV_O_RSV_O_FRA_O_YKA_O_ERL_O_ARL_O_PRA_O_FKG |
| 16665-94-3-D7 | NVF_O_YSV_O_RSV_O_FRA_O_YKA_O_ERL_O_ARL_O_PRA_O_FKG |
| 16665-94-3-D8 | NVF_O_YSV_O_RSV_O_FRA_O_YKA_O_ERL_O_ARL_O_PRA_O_FKG |
| 16665-94-3-D9 | NVF_O_YSV_O_RSV_O_FRA_O_YKA_O_ERL_O_ARL_O_PRA_O_FKG |
| 16665-94-3-D10 | NVF_O_YSV_O_RSV_O_FRA_O_YKA_O_ERL_O_ARL_O_PRA_O_FKG |
| 16665-94-3-D11 | NVF_O_YSV_O_RSV_O_FRA_O_YKA_O_ERL_O_ARL_O_PRA_O_FKG |
| 16665-94-3-D12 | NVF_O_YSV_O_RSV_O_FRA_O_YKA_O_ERL_O_ARL_O_PRA_O_FKG |
| 16665-94-3-E1 | NVF_O_YSV_O_RSV_O_FRA_O_YKA_O_ERL_O_ARL_O_PRA_O_FKG |
| 16665-94-3-E2 | NVF_O_YSV_O_RSV_O_FRA_O_YKA_O_ERL_O_ARL_O_PRA_O_FKG |
| 16665-94-3-E3 | NVF_O_YSV_O_RSV_O_FRA_O_YKA_O_ERL_O_ARL_O_PRA_O_FKG |
| 16665-94-3-E4 | NVF_O_YSV_O_RSV_O_FRA_O_YKA_O_ERL_O_ARL_O_PRA_O_FKG |
| 16665-94-3-E5 | NVF_O_YSV_O_RSV_O_FRA_O_YKA_O_ERL_O_ARL_O_PRA_O_FKG |
| 16665-94-3-E6 | NVF_O_YSV_O_RSV_O_FRA_O_YKA_O_ERL_O_ARL_O_PRA_O_FKG |
| 16665-94-3-E7 | NVF_O_YSV_O_RSV_O_FRA_O_YKA_O_ERL_O_ARL_O_PRA_O_FKG |
| 16665-94-3-E8 | NVF_O_YSV_O_RSV_O_FRA_O_YKA_O_ERL_O_ARL_O_PRA_O_FKG |
| 16665-94-3-E9 | NVF_O_YSV_O_RSV_O_FRA_O_YKA_O_ERL_O_ARL_O_PRA_O_FKG |
| 16665-94-3-E10 | NVF_O_YSV_O_RSV_O_FRA_O_YKA_O_ERL_O_ARL_O_PRA_O_FKG |

Table 3D shows sequences obtained from twenty-two individual beads synthesized with the same hit sequence from the NNP2 library. No false positive hits were observed for any of the ptychs, but one of the bead samples generated no sequence. For the ptych ONRF, only the D-asparagine deamidated form was detected and this is indicated by the lowercase “d” in the sequence.

TABLE 3D

| Sample-Vial | Sequence Read |
|---|---|
| 16665-94-4-G1 | RWF_O_LRL_O_FEV_O_KEL_O_AKF_O_NRFd_O_ARL_O_WKL_O_AVL |
| 16665-94-4-G2 | RWF_O_LRL_O_FEV_O_KEL_O_AKF_O_NRFd_O_ARL_O_WKL_O_AVL |
| 16665-94-4-G3 | RWF_O_LRL_O_FEV_O_KEL_O_AKF_O_NRFd_O_ARL_O_WKL_O_AVL |
| 16665-94-4-G4 | RWF_O_LRL_O_FEV_O_KEL_O_AKF_O_NRFd_O_ARL_O_WKL_O_AVL |
| 16665-94-4-G5 | RWF_O_LRL_O_FEV_O_KEL_O_AKF_O_NRFd_O_ARL_O_WKL_O_AVL |
| 16665-94-4-G7 | RWF_O_LRL_O_FEV_O_KEL_O_AKF_O_NRFd_O_ARL_O_WKL_O_AVL |
| 16665-94-4-G8 | RWF_O_LRL_O_FEV_O_KEL_O_AKF_O_NRFd_O_ARL_O_WKL_O_AVL |
| 16665-94-4-G9 | RWF_O_LRL_O_FEV_O_KEL_O_AKF_O_NRFd_O_ARL_O_WKL_O_AVL |
| 16665-94-4-G10 | RWF_O_LRL_O_FEV_O_KEL_O_AKF_O_NRFd_O_ARL_O_WKL_O_AVL |
| 16665-94-4-G11 | RWF_O_LRL_O_FEV_O_KEL_O_AKF_O_NRFd_O_ARL_O_WKL_O_AVL |
| 16665-94-4-G12 | RWF_O_LRL_O_FEV_O_KEL_O_AKF_O_NRFd_O_ARL_O_WKL_O_AVL |
| 16665-94-4-H1 | RWF_O_LRL_O_FEV_O_KEL_O_AKF_O_NRFd_O_ARL_O_WKL_O_AVL |
| 16665-94-4-H2 | RWF_O_LRL_O_FEV_O_KEL_O_AKF_O_NRFd_O_ARL_O_WKL_O_AVL |
| 16665-94-4-H3 | RWF_O_LRL_O_FEV_O_KEL_O_AKF_O_NRFd_O_ARL_O_WKL_O_AVL |
| 16665-94-4-H4 | RWF_O_LRL_O_FEV_O_KEL_O_AKF_O_NRFd_O_ARL_O_WKL_O_AVL |
| 16665-94-4-H5 | RWF_O_LRL_O_FEV_O_KEL_O_AKF_O_NRFd_O_ARL_O_WKL_O_AVL |
| 16665-94-4-H6 | RWF_O_LRL_O_FEV_O_KEL_O_AKF_O_NRFd_O_ARL_O_WKL_O_AVL |
| 16665-94-4-H7 | RWF_O_LRL_O_FEV_O_KEL_O_AKF_O_NRFd_O_ARL_O_WKL_O_AVL |
| 16665-94-4-H8 | RWF_O_LRL_O_FEV_O_KEL_O_AKF_O_NRFd_O_ARL_O_WKL_O_AVL |
| 16665-94-4-H9 | RWF_O_LRL_O_FEV_O_KEL_O_AKF_O_NRFd_O_ARL_O_WKL_O_AVL |
| 16665-94-4-H10 | RWF_O_LRL_O_FEV_O_KEL_O_AKF_O_NRFd_O_ARL_O_WKL_O_AVL |

Table 4 shows mass spectrometry validation of each hexaptych fragment from the NNP1 library. The ptych sequences are presented as single-letter amino acid codes; only those on the N-terminal side of the PAM-O-ester (M) are the natural L-configuration, and the remaining sites contain the corresponding unnatural D-stereoisomers.

TABLE 4

| | | Prominent | |
|---|---|---|---|
| Ptych | Monoisotopic | Neutral | |
| Position | m/z | Loss | Ptych Sequence Code |
| 1 | 683.3246 | 131.1 | _M_T_S_N_T_L |
| 1 | 686.3396 | 75.2 | _M_Y_L_S_V_G |
| 1 | 691.3773 | 278.2 | _M_L_P_R_T_G |
| 1 | 698.3396 | 89.2 | _M_S_P_Y_L_A |
| 1 | 703.3773 | 99 | _M_P_P_S_R_V |
| 1 | 706.3083 | 107.2 | _M_F_Y_S_A_A |
| 1 | 769.3515 | 328.1 | _M_Y_P_R_E_G |
| 1 | 387.6877 | 71 | _M_G_W_H_R_A |
| 1 | 774.3682 | 71 | _M_G_W_H_R_A |
| 1 | 395.1981 | 277.1 | _M_E_H_R_T_V |
| 1 | 789.389 | 277.1 | _M_E_H_R_T_V |
| 1 | 827.361 | 369.2 | _M_E_T_P_W_F |
| 1 | 830.3355 | 183.2 | _M_H_Y_S_E_F |
| 2 | 626.2668 | 93.1 | _M_S_V_S_E_G |
| 2 | 659.2671 | 165.2 | _M_S_S_G_N_F |
| 2 | 679.3661 | 348.3 | _M_A_E_L_K_A |
| 2 | 720.3464 | 324.1 | _M_P_G_R_W_G |
| 2 | 723.3824 | 418.2 | _M_R_F_P_V_G |
| 2 | 732.4039 | 99 | _M_P_R_V_N_V |
| 2 | 742.3294 | 204.3 | _M_P_Y_E_S_V |
| 2 | 744.3563 | 131.1 | _M_P_T_E_H_L |
| 2 | 787.3621 | 71 | _M_R_Y_T_E_A |
| 2 | 403.2158 | 113 | _M_K_Y_P_H_L |
| 2 | 805.4243 | 131 | _M_K_Y_P_H_L |
| 2 | 833.3828 | 131.1 | _M_E_T_H_W_L |
| 3 | 642.227 | 89.1 | _M_G_E_F_A_A |
| 3 | 651.3712 | 93.1 | _M_V_K_T_V_G |
| 3 | 687.262 | 75 | _M_G_E_N_Y_G |
| 3 | 696.3087 | 89.2 | _M_V_E_E_T_A |
| 3 | 697.3515 | 99 | _M_R_S_S_T_V |
| 3 | 364.1979 | 113 | _M_P_R_H_G_L |
| 3 | 727.3886 | 131 | _M_P_R_H_G_L |
| 3 | 741.393 | 131.2 | _M_N_P_L_H_L |
| 3 | 752.3977 | 93.1 | _M_T_K_W_L_G |
| 3 | 755.3723 | 71 | _M_Y_T_P_R_A |
| 3 | 838.4094 | 113.1 | _M_R_S_E_W_L |
| 3 | 429.7291 | 147.2 | _M_P_K_Y_R_F |
| 3 | 858.4508 | 147 | _M_P_K_Y_R_F |
| 4 | 605.2929 | 219.1 | _M_A_P_G_N_V |
| 4 | 672.3239 | 89.2 | _M_G_T_L_Y_A |
| 4 | 693.3202 | 75.1 | _M_P_S_E_R_G |
| 4 | 709.3767 | 149.1 | _M_K_T_E_A_L |
| 4 | 711.346 | 147 | _M_G_P_R_S_F |
| 4 | 713.3868 | 176.1 | _M_F_K_L_S_A |
| 4 | 735.3346 | 75.1 | _M_P_E_W_V_G |
| 4 | 372.1954 | 99 | _M_H_S_P_R_V |
| 4 | 743.3835 | 99 | _M_H_S_P_R_V |
| 4 | 753.3777 | 113.1 | _M_T_S_R_E_L |
| 4 | 416.211 | 316.4 | _M_H_N_K_W_V |
| 4 | 831.4184 | 17 | _M_H_N_K_W_V |
| 4 | 442.7061 | 211 | _M_Y_N_R_H_F |
| 4 | 884.4049 | 147 | _M_Y_N_R_H_F |
| 5 | 566.2457 | 89.1 | _M_P_S_G_S_A |
| 5 | 624.2875 | 75.1 | _M_G_L_E_T_G |
| 5 | 647.3511 | 71 | _M_G_V_P_R_A |
| 5 | 690.3345 | 149.1 | _M_P_S_P_E_L |
| 5 | 722.3508 | 131.1 | _M_W_A_N_A_L |
| 5 | 375.6821 | 147 | _M_G_K_H_N_F |
| 5 | 750.3569 | 17 | _M_G_K_H_N_F |
| 5 | 771.4287 | 131.1 | _M_T_Y_K_V_L |
| 5 | 790.3658 | 165.2 | _M_E_F_V_T_F |
| 5 | 829.3879 | 200.1 | _M_K_N_S_W_F |
| 5 | 420.7343 | 113 | _M_F_E_K_R_L |
| 5 | 840.4614 | 131.1 | _M_F_E_K_R_L |
| 5 | 861.4505 | 230.1 | _M_Y_Y_R_V_L |
| 6 | 448.2402 | 89.1 | S_T_V_A_A |
| 6 | 487.2875 | 188.1 | N_A_L_A_V |
| 6 | 570.2267 | 75.1 | N_N_E_H_G |
| 6 | 579.3361 | 325.2 | P_R_H_G_L |
| 6 | 588.2875 | 149.2 | E_P_T_E_L |
| 6 | 592.2977 | 234.1 | S_F_P_E_L |
| 6 | 592.2977 | 113.1 | L_P_E_S_F |
| 6 | 308.6399 | 63.6 | S_P_H_E_F |
| 6 | 616.2725 | 18 | S_P_H_E_F |
| 6 | 638.2933 | 165.11 | P_N_N_F_F |
| 6 | 351.1739 | 17 | W_P_H_Y_V |
| 6 | 701.3406 | 186.1 | W_P_H_Y_V |
| 6 | 701.3406 | 283.1 | Y_H_P_W_V |
| 6 | 351.1739 | 46 | Y_H_P_W_V |

Table 5 shows mass spectrometry validation of each tetraptych fragment from the NNP2 library. The ptych sequences are presented as single-letter amino acid codes; only those on the N-terminal side of the PAM-O-Ester (M) are the natural L-configuration, and the remaining sites contain the corresponding unnatural D-stereoisomers.

TABLE 5

| | | Prominent | |
|---|---|---|---|
| Ptych | Monoisotopic | Neutral | Ptych Sequence |
| Position | m/z | Loss | Code |
| 1 | 406.19732 | 89.1 | _O_P_A_A |
| 1 | 450.25982 | 219.1 | _O_A_V_L |
| 1 | 454.21842 | 249.1 | _O_T_S_V |
| 1 | 499.25512 | 295.1 | _O_F_K_G |
| 1 | 510.24462 | 249.1 | _O_T_E_L |
| 1 | 527.26122 | 138 | _O_F_R_G |
| 1 | 532.24002 | 246.1 | _O_H_E_V |
| 1 | 566.26092 | 117 | _O_H_Y_V |
| 1 | 581.26062 | 117.2 | _O_W_E_V |
| 1 | 633.30312 | 147 | _O_Y_R_F |
| 2 | 422.22862 | 219.1 | _O_A_L_A |
| 2 | 452.20282 | 135.1 | _O_G_E_V |
| 2 | 466.21842 | 149.1 | _O_G_E_L |
| 2 | 491.26132 | 71 | _O_P_R_A |
| 2 | 504.24522 | 218.1 | _O_H_S_L |
| 2 | 518.26092 | 131.1 | _O_T_H_L |
| 2 | 551.28242 | 99 | _O_E_R_V |
| 2 | 589.30206 | 165.1 | _O_K_F_F |
| 2 | 594.32862 | 334.2 | _O_W_K_L |
| 2 | 606.24462 | 277.1 | _O_E_Y_F |
| 3 | 420.21302 | 174.2 | _O_P_G_V |
| 3 | 468.23402 | 235.1 | _O_S_T_L |
| 3 | 472.20782 | 219.1 | _O_A_S_F |
| 3 | 494.24969 | 277.1 | _O_E_V_V |
| 3 | 507.29252 | 113.1 | _O_A_R_L |
| 3 | 522.29222 | 263.2 | _O_K_N_L |
| 3 | 546.25582 | 260.2 | _O_H_E_L |
| 3 | 558.24462 | 277.2 | _O_E_Y_V |
| 3 | 594.27622 | 277.1 | _O_E_W_L |
| 3 | 598.33482 | 165.1 | _O_R_K_F |
| 4 | 458.19212 | 219.1 | _O_A_Y_G |
| 4 | 482.21332 | 131.1 | _O_E_S_B |
| 4 | 486.22352 | 295.2 | _O_F_T_A |
| 4 | 488.20272 | 235 | _O_S_S_F |
| 4 | 540.23412 | 165.2 | _O_P_E_F |
| 4 | 553.22932 | 277.1 | _O_E_W_A |
| 4 | 565.29802 | 113.1 | _O_E_R_L |
| 4 | 579.29262 | 293.2 | _O_H_K_F |
| 4 | 584.28272 | 147 | _O_N_R_F |
| 4 | 585.2267 | 263.1 | _O_F_R_F |
| 4 | 599.31877 | 131.1 | _O_R_Y_L |
| 5 | 394.19732 | 205.1 | _O_G_A_V |
| 5 | 442.19732 | 219.1 | _O_A_G_V |
| 5 | 478.25482 | 131 | _O_P_T_L |
| 5 | 481.22922 | 262.1 | _O_N_S_L |
| 5 | 506.24972 | 131.2 | _O_P_E_L |
| 5 | 513.27082 | 219.1 | _O_A_K_F |
| 5 | 529.26572 | 311.1 | _O_Y_K_A |
| 5 | 545.30822 | 259.2 | _O_H_K_L |
| 5 | 571.27632 | 277.1 | _O_E_K_F |
| 5 | 608.31912 | 99 | _O_W_R_V |
| 6 | 36.16602 | 205.1 | _O_G_A_A |
| 6 | 440.20272 | 235.1 | _O_S_S_V |
| 6 | 496.22902 | 249.1 | _O_T_E_V |
| 6 | 519.29262 | 99 | _O_P_R_L |
| 6 | 537.29192 | 278.2 | _O_K_E_L |
| 6 | 541.27692 | 71 | _O_F_R_A |
| 6 | 555.31772 | 295.2 | _O_F_K_L |
| 6 | 564.28165 | 135.1 | _O_H_F_L |
| 6 | 591.24492 | 311.2 | _O_R_N_F |
| 6 | 622.33472 | 113.1 | _O_W_R_L |
| 7 | 463.25522 | 217.2 | _O_P_K_A |
| 7 | 490.19322 | 277.1 | _O_E_H_G |
| 7 | 495.24492 | 262.1 | _O_N_T_L |
| 7 | 500.20282 | 165.1 | _O_G_E_F |
| 7 | 521.33342 | 261.1 | _O_L_K_L |
| 7 | 535.32385 | 230.2 | _O_R_V_L |
| 7 | 539.21362 | 277.1 | _O_E_W_G |
| 7 | 542.24972 | 117.1 | _O_F_E_V |
| 7 | 557.27182 | 147 | _O_R_S_F |
| 7 | 557.27182 | 328.2 | _O_H_Y_F |
| 8 | 456.21292 | 219.1 | _O_A_A_F |
| 8 | 477.23442 | 117.1 | _O_P_N_V |
| 8 | 493.27692 | 113 | _O_G_R_L |
| 8 | 516.23402 | 311.1 | _O_Y_S_V |
| 8 | 530.21332 | 277.1 | _O_E_S_F |
| 8 | 544.26532 | 249.1 | O_T_Y_L |
| 8 | 549.33952 | 113.1 | _O_L_R_L |
| 8 | 550.33482 | 117 | _O_R_K_V |
| 8 | 561.24562 | 89 | _O_W_H_A |
| 8 | 585.30312 | 99 | _O_R_Y_V |
| 9 | 318.20229 | 131.2 | S_V_L |
| 9 | 359.26528 | 131.2 | K_V_L |
| 9 | 379.19759 | 165.2 | N_V_F |
| 9 | 380.21809 | 165.2 | T_L_F |
| 9 | 387.27149 | 99 | R_L_V |
| 9 | 396.17659 | 165.1 | E_T_F |
| 9 | 400.18669 | 235.1 | S_F_F |
| 9 | 408.24929 | 131.2 | Y_L_L |
| 9 | 424.20779 | 131.25 | Y_E_L |
| 9 | 508.26669 | 165.1 | R_W_F |

Table 6 shows K-Ras binding assay titration against NNP1.

TABLE 6

| Well number | 1 | 2 | 3 | 4 | 5 | 6 | 7 | 8 |
|---|---|---|---|---|---|---|---|---|
| Probe | KRAS4B- | KRAS4B- | KRAS4B- | KRAS4B- | KRAS4B- | KRAS4B- | KRAS4B- | KRAS4B- |
| | CF555 | CF555 | CF555 | CF555 | CF555 | CF555 | CF555 | CF555 |
| Probe | 150 | 75 | 37.5 | 18.8 | 9.4 | 150 | 0 | 0 |
| conc. (nM) | | | | | | | | |
| Anti-probe | RAF- | RAF- | RAF- | RAF- | RAF- | RAF- | RAF- | RAF- |
| | RBD- | RBD- | RBD- | RBD- | RBD- | RBD- | RBD- | RBD- |
| | 647 | 647 | 647 | 647 | 647 | 647 | 647 | 647 |
| Anti-probe | 600 | 300 | 150 | 75 | 37.5 | 600 | 0 | 0 |
| conc. (nM) | | | | | | | | |
| Number of | 100K | 100K | 100K | 100K | 100K | 100K | 90K | 100K |
| beads | | | | | | | | |
| Type of | NNP1 | NNP1 | NNP1 | NNP1 | NNP1 | 20 μm | 20 μm | NNP1 |
| bead | Library | Library | Library | Library | Library | naked | naked | |
| | | | | | | beads | beads | |
| 2nd type of | | | | | | 10K | 10K | |
| bead | | | | | | 30 μm | 30 μm | |
| | | | | | | peptide | peptide | |
| | | | | | | beads | beads | |
| FAST | F1-50/ | F1-50/ | F1-50/ | F1-50/ | F1-50/ | F1-50/ | F1-50/ | F1-50/ |
| filters used | F1-400 | F1-400 | F1-400 | F1-400 | F1-400 | F1-400 | F1-400 | F1-400 |
| Number of | 4400/500 | 1700/50 | 350/3 | 200/0 | 16/0 | many/2 | 14/2 | 40/0 |
| FAST hits | | | | | | | | |
| (by filter) | | | | | | | | |
| Grey value | 15,500 | 15,250 | 14,500 | NA | NA | NA | NA | NA |
| mean of | | | | | | | | |
| TPs (555) | | | | | | | | |
| Grey value | 2600 | 2000 | 1500 | | | | | |
| mean of | | | | | | | | |
| TPs (647) | | | | | | | | |
| Ratio | 6 | 8 | 10 | | | | | |
| (555/647): | | | | | | | | |

Table 7 shows ASGPR binding assay titration against NNP1.

TABLE 7

| Well number | 1 | 2 | 3 | 4 | 5 | 6 | 7 | 8 |
|---|---|---|---|---|---|---|---|---|
| Probe | ASGPR- | ASGPR- | ASGPR- | ASGPR- | ASGPR- | ASGPR- | ASGPR- | ASGPR- |
| | CF555 | CF555 | CF555 | CF555 | CF555 | CF555 | CF555 | CF555 |
| Conc. (nM) | 150 | 75 | 37.5 | 18.75 | 9.3 | 4.7 | 150 | 0 |
| Number of beads | 50k | 50k | 50k | 50k | 50k | 50k | 50k | 50K |
| Type of bead | NNP1 | NNP1 | NNP1 | NNP1 | NNP1 | NNP1 | Naked | NNP1 |
| | | | | | | | (20 μm) | |
| FAST filters | F1-6000 | F1-6000 | F1-6000 | F1-6000/ | F1-3000 | F1-3000 | F1-3000 | F1-3000 |
| used | | | | F1-3000 | | | | |
| Number of FAST | 1100 | 500 | 100 | 25/550 | 50 | 10 | 0 | 0 |
| hits | | | | | | | | |
| True positives | NA | 500 | 100 | 15/350 | 40 | 10 | 0 | 0 |
| (TPs) | | | | | | | | |
| Grey value | 16,000 | 16,000 | 13,000 | 12,000 | NA | NA | NA | NA |
| mean of | | | | | | | | |
| TPs (5 ms) | | | | | | | | |

Table 8 shows IL-6 binding assay titration against NNP2.

TABLE 8

| Well number | 1 | 2 | 3 | 4 | 5 |
|---|---|---|---|---|---|
| Probe | IL6- | IL6- | IL6- | IL6- | IL6- |
| | AF555 | AF555 | AF555 | AF555 | AF555 |
| Probe conditions | 75 | 37.5 | 18.75 | 9.3 | 0 |
| (nM) | | | | | |
| Anti-probe | IL6R- | IL6R- | IL6R- | IL6R- | IL6R- |
| | AF647 | AF647 | AF647 | AF647 | AF647 |
| Anti-probe | 300 | 150 | 75 | 37.5 | 0 |
| conditions (nM) | | | | | |
| Number of beads | 100K | 100K | 100K | 100K | 100K |
| Type of bead | NNP2 | NNP2 | NNP2 | NNP2 | NNP2 |
| FAST filters used | F1-15 | F1-15 | F1-15 | F1-15 | F1-15 |
| Number of FAST hits | 32 | 45 | 55 | 59 | 64 |
| True positives (TPs) | 4 | 1 | 2 | 2 | 0 |
| IL6+/IL6R− (+/−) | 0 | 0 | 2 | 2 | 0 |
| IL6+/IL6R+ (+/+) | 4 | 1 | 0 | 0 | 0 |
| Grey value mean of | 10-13K | 12K | 7-11K | 5-6K | NA |
| TPs (5 ms) | | | | | |
| Ratio (AF555/AF647) | 0.91-1.12 | 0.82 | NA | NA | NA |

Table 9 shows IL-6R binding assay titration against NNP2.

TABLE 9

| Well number | 1 | 2 | 3 | 4 | 5 | 6 | 8 |
|---|---|---|---|---|---|---|---|
| Probe | IL6R- | IL6R- | IL6R- | IL6R- | IL6R- | IL6R- | IL6R- |
| | AF555 | AF555 | AF555 | AF555 | AF555 | AF555 | AF555 |
| Probe conditions | 18.8 | 9.4 | 4.7 | 2.4 | 1.2 | 0 | 18.8 |
| (nM) | | | | | | | |
| Anti-probe | IL6-AF647 | IL6-AF647 | IL6-AF647 | IL6-AF647 | IL6-AF647 | IL6-AF647 | IL6-AF647 |
| Anti-probe | 75 | 37.5 | 18.8 | 9.4 | 4.7 | 0 | 0 |
| conditions (nM) | | | | | | | |
| Number of beads | 100k | 100k | 100k | 100k | 100k | 100k | 100k |
| Type of bead | NNP2 | NNP2 | NNP2 | NNP2 | NNP2 | NNP2 | NNP2 |
| FAST filters used | F1-8 | F1-8 | F1-8 | F1-8 | F1-8 | F1-8 | F1-8 |
| Number of FAST | 111 | 66 | 54 | 29 | 4 | 10 | 25 |
| hits | | | | | | | |
| True positives | 52 | 22 | 7 | 0 | 0 | 0 | 4 |
| (TPs) | | | | | | | |
| Probe+/Anti- | 0 | 0 | 0 | 0 | 0 | 0 | NA |
| probe− (+/−) | | | | | | | |
| Probe+/Anti- | 52 | 22 | 7 | 0 | 0 | 0 | NA |
| probe+ (+/+) | | | | | | | |
| Grey value mean | 3-14K | 5-10K | 3-6K | NA | NA | NA | 4-12K |
| of TPs (red) | | | | | | | |
| Ratio | 0.41-2.89 | 0.4-0.67 | 0.38-0.44 | NA | NA | NA | NA |

Table 10 shows TNFa binding assay titration against NNP2. ARI=average red intensity.

TABLE 10

| Well number | 1 | 2 | 3 | 4 | 5 | 6 | 7 | 8 |
|---|---|---|---|---|---|---|---|---|
| Probe | TNFα- | TNFα- | TNFα- | TNFα- | TNFα- | TNFα- | TNFα- | TNFα- |
| | CF555 | CF555 | CF555 | CF555 | CF555 | CF555 | CF555 | CF555 |
| Probe conditions | 100 | 50 | 25 | 12.5 | 6.3 | 3.1 | 0 | 100.0 |
| (nM) | | | | | | | | |
| Number of beads | 250K | 250K | 250K | 250K | 250K | 250K | 250K | 250K |
| NNP2 | NNP2 | NNP2 | NNP2 | NNP2 | NNP2 | NNP2 | NNP2 | 10 μm |
| | | | | | | | | Naked |
| FAST filters | ARI = 40 | ARI = 40 | ARI = 40 | ARI = 40 | ARI = 40 | ARI = 40 | ARI = 40 | ARI = 40 |
| used | | | | | | | | |
| Number of | 30 | 18 | 42 | 48 | 26 | 7 | 22 | 5/5 |
| FAST hits | | | | | | | | |
| True positives | NA | NA | NA | NA | NA | NA | NA | NA |
| (TPs) | | | | | | | | |
| Grey value | 10-12K | 10-12K | 10-12K | 10-12K | 10-12K | 10-12K | 10-12K | NA |
| mean of TPs | | | | | | | | |
| (555) | | | | | | | | |

Table 11 illustrates optimized conditions of the binding assay for individual targets and library.

TABLE 11

| | | | Binding | | | | | |
|---|---|---|---|---|---|---|---|---|
| | | | steps for | | | | 1^{st} | 2^{nd} |
| | | | dual | Rehyd. | Blocking | Binding | Washing | Washing |
| Target | Libr. | Probe | probe | buffer | buffer | buffer | buffer | buffer |
| Kras | NNP1 | Dual (K- | Sequential | 1% PEG, 50 mM | 100% Odyssey | 50% Odyssey, | 0.5% chaps, | 0.5% chaps, |
| | | Ras4B-CF555 | | Tris, pH 7.5, | buffer/0.5% | 0.5% chaps, | 200 mM NaCl, | 200 mM NaCl, |
| | | 75 nM/RAF- | | 1% BSA | chaps, 200 mM | 200 mM NaCl, | 5 mM MgCl2, | 5 mM MgCl2, |
| | | RBD-AF 647, | | | NaCl in TBS | 5 mM MgCl2, | 1 mM TCEP, | 1 mM TCEP, |
| | | 600 nM) | | | | 1 mM TCEP, | 10 μm GTPyS | 10 μm GTPyS |
| | | | | | | 10 μM GTPyS | in TBS | in TBS |
| | | | | | | in TBS50% | | |
| | | | | | | Odyssey buffer/ | | |
| | | | | | | 0.5% chaps, | | |
| | | | | | | 200 mM NaCl | | |
| | | | | | | in PBS | | |
| ASGPR | NNP1 | Mono-Mono- | N/A | 1% PEG, 50 mM | 100% Odyssey | 1 mM CaCl2, | 1 mM CaCl2, | 0.5% chaps |
| | | (ASGPR-CF555, | | Tris, pH 7.5, | buffer/0.5% | 0.5 mM MgSO4, | 0.5 mM MgSO4, | in PBS |
| | | 10 nM | | 1% BSA | chaps, 200 mM | 0.5% chaps, | 0.5% chaps, | |
| | | | | | NaCl in PBS | and 200 mM | and 200 mM | |
| | | | | | | NaCl in PBS | NaCl in PBS | |
| IL-6 | NNP2 | Dual (IL6- | Complex, | 1% PEG, 50 mM | 50% Odyssey | 50% Odyssey | 0.5% chaps, | 0.5% chaps |
| | | AF555 18.8 nM/ | Simult. | Tris, pH 7.5, | buffer/0.5% | buffer/0.5% | 200 mM NaCl | in PBS |
| | | ILR6R-AF647, | | 1% BSA | chaps, 200 mM | chaps, 200 mM | in PBS | |
| | | 75 nM) | | | NaCl in PBS | NaCl in PBS | | |
| IL-6R | NNP2 | Dual (IL6R- | Sequential | 1% PEG, 50 mM | 100% Odyssey | 50% Odyssey | 0.5% chaps, | 0.5% chaps |
| | | AF555 18.8 nM/ | | Tris, pH 7.5, | buffer/0.5% | buffer/0.5% | 200 mM NaCl | in PBS |
| | | ILR6-AF647, | | 1% BSA | chaps, 200 mM | chaps, 200 mM | in PBS | |
| | | 75 nM) | | | NaCl in PBS | NaCl in PBS | | |
| TNFα | NNP2 | Mono-(TNF- | N/A | 1% PEG, 50 mM | 100% Odyssey | 50% Odyssey | 0.5% chaps, | 0.5% chaps |
| | | alpha CF555, | | Tris, pH 7.5, | buffer/0.5% | buffer/0.5% | 200 mM NaCl | in PBS |
| | | 100 nM) | | 1% BSA | chaps, 200 mM | chaps, 200 mM | in PBS | |
| | | | | | NaCl in PBS | NaCl in PBS | | |

Table 12 shows a comparison of binding affinities of ester versus amide replacement polymers to IL-6R.

TABLE 12

| | Amino | | MST (microscale |
|---|---|---|---|
| | Acid-PAM | | thermophoresis) |
| Identifier | Bond | Library | K_{D} (nM) [95% conf. int] |
| Hit 1 (IL6R-93-12) | Ester | NNP2 | 3.7 [0.82-14.1] |
| Hit 1 (IL6R-87-8) | Amide | NNP2 | 0.62 [0.34-1.1] |
| Hit 2 (IL6R-93-13) | Ester | NNP2 | 130 [57-460] |
| Hit 2 (IL6R-87-10) | Amide | NNP2 | 20 [5.4-67] |

Table 13 shows a summary of NNP hits against various targets and the measured binding affinities by microscale thermophoresis (MST). K_{D} values were all determined by fitting data to a one-site binding model by performing a nonlinear regression fitting with GraphPad Prism 8.3.1.

TABLE 13

| | | | | KD (nM) |
|---|---|---|---|---|
| | | | | [95% conf. |
| Identifier | Sequence | Library | Target | int] |
| KRAS-1-1 | Fluorescein-(D)Trp-(D)Pro-(D)His- | NNP1 | K-Ras | 24 |
| | (D)Tyr-(L)Val-PAM-Gly-(D)Val- | | | [11 - 47] |
| | (D)Pro-(D)Arg-(L)Ala-PAM-(D)Pro- | | | |
| | (D)Ser-(D)Glu-(D)Arg-Gly-PAM- | | | |
| | (D)Pro-(D)Lys-(D)Tyr-(D)Arg-(L)Phe- | | | |
| | PAM-(D)Ser-(D)Ser-Gly-(D)Asn- | | | |
| | (L)Phe-PAM-Gly-(D)Trp-(D)His- | | | |
| | (D)Arg-(L)Ala-PAM | | | |
| KRAS-1-2 | Fluorescein-(D)Trp-(D)Pro-(D)His- | NNP1 | K-Ras | 49 |
| | (D)Tyr-(L)Val-PAM-Gly-(D)Val- | | | [24 - 93] |
| | (D)Pro-(D)Arg-(L)Ala-PAM-(D)Pro- | | | |
| | (D)Ser-(D)Glu-(D)Arg-Gly-PAM- | | | |
| | (D)Pro-(D)Lys-(D)Tyr-(D)Arg-(L)Phe- | | | |
| | PAM-(D)Pro-Gly-(D)Arg-(D)Trp-Gly- | | | |
| | PAM-(D)Tyr-(D)Pro-(D)Arg-(D)Glu- | | | |
| | Gly-PAM | | | |
| KRAS-1-3 | Fluorescein-(D)Leu-(D)Pro-(D)Glu- | NNP1 | K-Ras | 18 |
| | (D)Ser-(L)Phe-PAM-Gly-(D)Val- | | | [5 - 31] |
| | (D)Pro-(D)Arg-(L)Ala-PAM-(D)Pro- | | | |
| | (D)Ser-(D)Glu-(D)Arg-Gly-PAM- | | | |
| | (D)Pro-(D)Lys-(D)Tyr-(D)Arg-(L)Phe- | | | |
| | PAM-(D)Arg-(D)Tyr-(D)Thr-(D)Glu- | | | |
| | (L)Ala-PAM-Gly-(D)Trp-(D)His- | | | |
| | (D)Arg-(L)Ala-PAM | | | |
| KRAS-1-4 | Fluorescein-(D)Leu-(D)Pro-(D)Glu- | NNP1 | K-Ras | 36 |
| | (D)Ser-(L)Phe-PAM-Gly-(D)Val- | | | [10 - 97] |
| | (D)Pro-(D)Arg-(L)Ala-PAM-(D)Pro- | | | |
| | (D)Ser-(D)Glu-(D)Arg-Gly-PAM- | | | |
| | (D)Pro-(D)Lys-(D)Tyr-(D)Arg-(L)Phe- | | | |
| | PAM-(D)Arg-(D)Tyr-(D)Thr-(D)Glu- | | | |
| | (L)Ala-PAM-(D)Tyr-(D)Pro-(D)Arg- | | | |
| | (D)Glu-Gly-PAM | | | |
| KRAS-1-5 | Fluorescein-(D)Trp-(D)Pro-(D)His- | NNP1 | K-Ras | 75 |
| | (D)Tyr-(L)Val-PAM-(D)Pro-(D)Ser- | | | [22 - 230] |
| | Gly-(D)Ser-(L)Ala-PAM-(D)Pro- | | | |
| | (D)Ser-(D)Glu-(D)Arg-Gly-PAM- | | | |
| | (D)Pro-(D)Lys-(D)Tyr-(D)Arg-(L)Phe- | | | |
| | PAM-(D)Pro-Gly-(D)Arg-(D)Trp-Gly- | | | |
| | PAM-(D)Tyr-(D)Pro-(D)Arg-(D)Glu- | | | |
| | Gly-PAM | | | |
| KRAS-1-6 | Fluorescein-(D)Ser-(D)Pro-(D)His- | NNP1 | K-Ras | 21 |
| | (D)Glu-(L)Phe-PAM-(D)Pro-(D)Ser- | | | [6.3 - 57] |
| | Gly-(D)Ser-(L)Ala-PAM-(D)Thr- | | | |
| | (D)Ser-(D)Arg-(D)Glu-(L)Leu-PAM- | | | |
| | (D)Pro-(D)Lys-(D)Tyr-(D)Arg-(L)Phe- | | | |
| | PAM-(D)Arg-(D)Tyr-(D)Thr-(D)Glu- | | | |
| | (L)Ala-PAM-Gly-(D)Trp-(D)His- | | | |
| | (D)Arg-(L)Ala-PAM | | | |
| KRAS-1-8 | Fluorescein-(D)Trp-(D)Pro-(D)His- | NNP1 | K-Ras | 44 |
| | (D)Tyr-(L)Val-PAM-Gly-(D)Val- | | | [19 - 93] |
| | (D)Pro-(D)Arg-(L)Ala-PAM-Gly- | | | |
| | (D)Pro-(D)Arg-(D)Ser-(L)Phe-PAM- | | | |
| | (D)Tyr-(D)Thr-(D)Pro-(D)Arg-(L)Ala- | | | |
| | PAM-(D)Pro-Gly-(D)Arg-(D)Trp-Gly- | | | |
| | PAM-(D)Ser-(D)Pro-(D)Tyr-(D)Leu- | | | |
| | (L)Ala-PAM | | | |
| KRAS-1-9 | Fluorescein-(D)Ser-(D)Phe-(D)Pro- | NNP1 | K-Ras | 32 |
| | (D)Glu-(L)Leu-PAM-Gly-(D)Val- | | | [8.8 - 106] |
| | (D)Pro-(D)Arg-(L)Ala-PAM-(D)Pro- | | | |
| | (D)Ser-(D)Glu-(D)Arg-Gly-PAM- | | | |
| | (D)Pro-(D)Lys-(D)Tyr-(D)Arg-(L)Phe- | | | |
| | PAM-(D)Arg-(D)Phe-(D)Pro-(D)Val- | | | |
| | Gly-PAM-Gly-(D)Trp-(D)His-(D)Arg- | | | |
| | (L)Ala-PAM | | | |
| KRAS-1-10 | Fluorescein-(D)Glu-(D)Pro-(D)Thr- | NNP1 | K-Ras | 60 |
| | (D)Glu-(L)Leu-PAM-Gly-(D)Val- | | | [22 - 98] |
| | (D)Pro-(D)Arg-(L)Ala-PAM-(D)Pro- | | | |
| | (D)Ser-(D)Glu-(D)Arg-Gly-PAM- | | | |
| | (D)Pro-(D)Lys-(D)Tyr-(D)Arg-(L)Phe- | | | |
| | PAM-(D)Arg-(D)Tyr-(D)Thr-(D)Glu- | | | |
| | (L)Ala-PAM-Gly-(D)Trp-(D)His- | | | |
| | (D)Arg-(L)Ala-PAM | | | |
| KRAS-1-11 | Fluorescein-(D)Tyr-(D)His-(D)Pro- | NNP1 | K-Ras | 60 |
| | (D)Trp-(L)Val-PAM-Gly-(D)Lys- | | | [23 - 140] |
| | (D)His-(D)Asn-(L)Phe-PAM-(D)Pro- | | | |
| | (D)Ser-(D)Glu-(D)Arg-Gly-PAM- | | | |
| | (D)Pro-(D)Lys-(D)Tyr-(D)Arg-(L)Phe- | | | |
| | PAM-(D)Pro-Gly-(D)Arg-(D)Trp-Gly- | | | |
| | PAM-(D)Tyr-(D)Pro-(D)Arg-(D)Glu- | | | |
| | Gly-PAM | | | |
| KRAS-1-12 | Fluorescein-(D)Leu-(D)Pro-(D)Glu- | NNP1 | K-Ras | 24 |
| | (D)Ser-(L)Phe-PAM-(D)Phe-(D)Glu- | | | [14 - 40] |
| | (D)Lys-(D)Arg-(L)Leu-PAM-(D)Pro- | | | |
| | (D)Ser-(D)Glu-(D)Arg-Gly-PAM- | | | |
| | (D)Pro-(D)Lys-(D)Tyr-(D)Arg-(L)Phe- | | | |
| | PAM-(D)Pro-Gly-(D)Arg-(D)Trp-Gly- | | | |
| | PAM-Gly-(D)Trp-(D)His-(D)Arg- | | | |
| | (L)Ala-PAM | | | |
| KRAS-1-13 | Fluorescein-(D)Leu-(D)Pro-(D)Glu- | NNP1 | K-Ras | 30 |
| | (D)Ser-(L)Phe-PAM-Gly-(D)Val- | | | [13- 66] |
| | (D)Pro-(D)Arg-(L)Ala-PAM-(D)Lys- | | | |
| | (D)Thr-(D)Glu-(D)Ala-(L)Leu-PAM- | | | |
| | (D)Tyr-(D)Thr-(D)Pro-(D)Arg-(L)Ala- | | | |
| | PAM-(D)Pro-Gly-(D)Arg-(D)Trp-Gly- | | | |
| | PAM-Gly-(D)Trp-(D)His-(D)Arg- | | | |
| | (L)Ala-PAM | | | |
| KRAS-1-14 | Fluorescein-(D)Ser-(D)Pro-(D)His- | NNP1 | K-Ras | 155 |
| | (D)Glu-(L)Phe-PAM-Gly-(D)Val- | | | [44 - 270] |
| | (D)Pro-(D)Arg-(L)Ala-PAM-(D)Pro- | | | |
| | (D)Ser-(D)Glu-(D)Arg-Gly-PAM- | | | |
| | (D)Pro-(D)Lys-(D)Tyr-(D)Arg-(L)Phe- | | | |
| | PAM-(D)Ala-(D)Glu-(D)Leu-(D)Lys- | | | |
| | (L)Ala-PAM-Gly-(D)Trp-(D)His- | | | |
| | (D)Arg-(L)Ala-PAM | | | |
| KRAS-1-15 | Fluorescein-(D)Trp-(D)Pro-(D)His- | NNP1 | K-Ras | 180 |
| | (D)Tyr-(L)Val-PAM-(D)Phe-(D)Glu- | | | [17 - |
| | (D)Lys-(D)Arg-(L)Leu-PAM-(D)Pro- | | | 4,900] |
| | (D)Ser-(D)Glu-(D)Arg-Gly-PAM- | | | |
| | (D)Pro-(D)Lys-(D)Tyr-(D)Arg-(L)Phe- | | | |
| | PAM-(D)Pro-Gly-(D)Arg-(D)Trp-Gly- | | | |
| | PAM-(D)His-(D)Tyr-(D)Ser-(D)Glu- | | | |
| | (L)Phe-PAM | | | |
| ASGPR-9-3 | Fluorescein-(D)Leu-(D)Pro-(D)Glu- | NNP1 | ASGPR | 330 |
| | (D)Ser-(L)Phe-PAM-(D)Phe-(D)Glu- | | | [220 - 440] |
| | (D)Lys-(D)Arg-(L)Leu-PAM-Gly- | | | |
| | (D)Pro-(D)Arg-(D)Ser-(L)Phe-PAM- | | | |
| | (D)Pro-(D)Lys-(D)Tyr-(D)Arg-(L)Phe- | | | |
| | PAM-(D)Arg-(D)Phe-(D)Pro-(D)Val- | | | |
| | Gly-PAM-(D)Leu-(D)Pro-(D)Arg- | | | |
| | (D)Thr-Gly-PAM | | | |
| ASGPR-9-4 | Fluorescein-(D)Leu-(D)-Pro-(D)Glu- | NNP1 | ASGPR | 230 |
| | (D)Ser-(L)Phe-PAM-(D)Phe-(D)Glu- | | | [10 - 450] |
| | (D)Lys-(D)Arg-(L)Leu-PAM-Gly- | | | |
| | (D)Pro-(D)Arg-(D)Ser-(L)Phe-PAM- | | | |
| | (D)Pro-(D)Arg-(D)His-Gly-(L)Leu- | | | |
| | PAM-(D)Arg-(D)Phe-(D)Pro-(D)Val- | | | |
| | Gly-PAM-(D)Leu-(D)Pro-(D)Arg- | | | |
| | (D)Thr-Gly-PAM | | | |
| ASGPR-9-6 | Fluorescein-(D)Leu-(D)-Pro-(D)Glu- | NNP1 | ASGPR | 34 |
| | (D)Ser-(L)Phe-PAM-(D)Phe-(D)Glu- | | | [11 - 94] |
| | (D)Lys-(D)Arg-(L)Leu-PAM-Gly- | | | |
| | (D)Pro-(D)Arg-(D)Ser-(L)Phe-PAM- | | | |
| | (D)Tyr-(D)Thr-(D)Pro-(D)Arg-(L)Ala- | | | |
| | PAM-(D)Arg-(D)Phe-(D)Pro-(D)Val- | | | |
| | Gly-PAM-(D)Leu-(D)Pro-(D)Arg- | | | |
| | (D)Thr-Gly-PAM | | | |
| ASGPR-9-7 | Fluorescein-(D)Leu-(D)Pro-(D)Glu- | NNP1 | ASGPR | 150 |
| | (D)Ser-(L)Phe-PAM-(D)Phe-(D)Glu- | | | [110 - 210] |
| | (D)Lys-(D)Arg-(L)Leu-PAM-(D)His- | | | |
| | (D)Ser-(D)Pro-(D)Arg-(L)Val-PAM- | | | |
| | (D)Pro-(D)Lys-(D)Tyr-(D)Arg-(L)Phe- | | | |
| | PAM-(D)Arg-(D)Phe-(D)Pro-(D)Val- | | | |
| | Gly-PAM-(D)Leu-(D)Pro-(D)Arg- | | | |
| | (D)Thr-Gly-PAM | | | |
| ASGPR-9- | Fluorescein-(D)Leu-(D)Pro-(D)Glu- | NNP1 | ASGPR | 97 |
| 10 | (D)Ser-(L)Phe-PAM-(D)Phe-(D)Glu- | | | [39 - 310] |
| | (D)Lys-(D)Arg-(L)Leu-PAM-(D)Tyr- | | | |
| | (D)Asn-(D)Arg-(D)His-(L)Phe-PAM- | | | |
| | (D)Asn-(D)Pro-(D)Leu-(D)His-(L)Leu- | | | |
| | PAM-(D)Arg-(D)Phe-(D)Pro-(D)Val- | | | |
| | Gly-PAM-(D)Leu-(D)Pro-(D)Arg- | | | |
| | (D)Thr-Gly-PAM | | | |
| ASGPR-9- | Fluorescein-(D)Leu-(D)Pro-(D)Glu- | NNP1 | ASGPR | 0.22 |
| 11 | (D)Ser-(L)Phe-PAM-(D)Phe-(D)Glu- | | | [0.072 - |
| | (D)Lys-(D)Arg-(L)Leu-PAM-(D)Tyr- | | | 0.44] |
| | (D)Asn-(D)Arg-(D)His-(L)Phe-PAM- | | | |
| | (D)Arg-(D)Ser-(D)Glu-(D)Trp-(L)Leu- | | | |
| | PAM-(D)Arg-(D)Phe-(D)Pro-(D)Val- | | | |
| | Gly-PAM-(D)Leu-(D)Pro-(D)Arg- | | | |
| | (D)Thr-Gly-PAM | | | |
| ASGPR-9- | Fluorescein-(D)Leu-(D)Pro-(D)Glu- | NNP1 | ASGPR | 6.0 |
| 12 | (D)Ser-(L)Phe-PAM-(D)Phe-(D)Glu- | | | [3.6 - 10] |
| | (D)Lys-(D)Arg-(L)Leu-PAM-(D)Tyr- | | | |
| | (D)Asn-(D)Arg-(D)His-(L)Phe-PAM- | | | |
| | (D)Tyr-(D)Thr-(D)Pro-(D)Arg-(L)Ala- | | | |
| | PAM-(D)Arg-(D)Phe-(D)Pro-(D)Val- | | | |
| | Gly-PAM-(D)Leu-(D)Pro-(D)Arg- | | | |
| | (D)Thr-Gly-PAM | | | |
| ASGPR-9- | Fluorescein-(D)Leu-(D)Pro-(D)Glu- | NNP1 | ASGPR | 20 |
| 14 | (D)Ser-(L)Phe-PAM-Gly-(D)Val- | | | [12 - 32] |
| | (D)Pro-(D)Arg-(L)Ala-PAM-Gly- | | | |
| | (D)Pro-(D)Arg-(D)Ser-(L)Phe-PAM- | | | |
| | (D)Pro-(D)Arg-(D)His-Gly-(L)Leu- | | | |
| | PAM-(D)Arg-(D)Phe-(D)Pro-(D)Val- | | | |
| | Gly-PAM-(D)Leu-(D)Pro-(D)Arg- | | | |
| | (D)Thr-Gly-PAM | | | |
| IL6-65-1 | Fluorescein-(D)Tyr--(D)Leu-(L)Leu- | NNP2 | IL-6 | 500 |
| | PAM-(D)Tyr-(D)Ser-(L)Val-PAM- | | | [300 - |
| | (D)Arg-(D)Ser-(L)Phe-PAM-(D)Trp- | | | 1,200] |
| | (D)Arg-(L)Leu-PAM-(D)Trp-(D)Arg- | | | |
| | (L)Val-PAM-(D)Arg-(D)Tyr-(L)Leu- | | | |
| | PAM-(D)Glu-(D)Tyr-(L)Val-PAM- | | | |
| | (D)Lys-(D)Phe-(L)Phe-PAM-(D)Tyr- | | | |
| | (D)Arg-(L)Phe-PAM | | | |
| IL6-65-2 | Fluorescein-(D)His-(D)Leu-(L)Phe- | NNP2 | IL-6 | 400 |
| | PAM-(D)Ala-(D)Ala-(L)Phe-PAM- | | | [100 - |
| | (D)Pro-(D)Lys-(L)Ala-PAM-(D)Phe- | | | 1,200] |
| | (D)Arg-(L)Ala-PAM-(D)Ala-(D)Lys- | | | |
| | (L)Phe-PAM-(D)Phe-(D)Thr-(L)Ala- | | | |
| | PAM-(D)Lys-(D)Asn-(L)Leu-PAM- | | | |
| | (D)Thr-(D)His-(L)Leu-PAM-(D)Phe- | | | |
| | (D)Lys-Gly-PAM | | | |
| IL6-65-3 | Fluorescein-(D)Arg-(D)Trp-(L)Phe- | NNP2 | IL-6 | 90 |
| | PAM-(D)Glu-(D)Ser-(L)Phe-PAM- | | | [30 - 450] |
| | (D)Arg-(D)Ser-(L)Phe-PAM-(D)Trp- | | | |
| | (D)Arg-(L)Leu-PAM-Gly-(D)Ala- | | | |
| | (L)Val-PAM-(D)Asn-(D)Arg-(L)Phe- | | | |
| | PAM-(D)Ala-(D)Ser-(L)Phe-PAM- | | | |
| | (D)Pro-(D)Arg-(L)Ala-PAM-(D)Phe- | | | |
| | (D)Lys-Gly-PAM | | | |
| IL6-65-4 | Fluorescein-(D)Arg-(D)Trp-(L)Phe- | NNP2 | IL-6 | 37 |
| | PAM-(D)Leu-(D)Arg-(L)Leu-PAM- | | | [8.3 - 37] |
| | (D)His-(D)Tyr-(L)Phe-PAM-(D)Trp- | | | |
| | (D)Arg-(L)Leu-PAM-(D)His-(D)Lys- | | | |
| | (L)Leu-PAM-(D)Ala-(D)Tyr-Gly- | | | |
| | PAM-(D)His-(D)Glu-(L)Leu-PAM- | | | |
| | (D)Pro-(D)Arg-(L)Ala-PAM-(D)Phe- | | | |
| | (D)Lys-Gly-PAM | | | |
| IL6-65-5 | Fluorescein-(D)Ser-(D)Phe-(L)Phe- | NNP2 | IL-6 | 33 |
| | PAM-Gly-(D)Arg-(L)Leu-PAM- | | | [10- 160] |
| | (D)Leu-(D)Lys-(L)Leu-PAM-(D)Trp- | | | |
| | (D)Arg-(L)Leu-PAM-(D)Ala-(D)Lys- | | | |
| | (L)Phe-PAM-(D)His-(D)Lys-(L)Phe- | | | |
| | PAM-(D)Ala-(D)Arg-(L)Leu-PAM- | | | |
| | (D)Thr-(D)His-(L)Leu-PAM-(D)Phe- | | | |
| | (D)Arg-Gly-PAM | | | |
| IL6-65-7 | Fluorescein-(D)Tyr-(D)Leu-(L)Leu- | NNP2 | IL-6 | 210 |
| | PAM-Gly-(D)Arg-(L)Leu-PAM- | | | [130- |
| | (D)Arg-(D)Ser-(L)Phe-PAM-(D)Phe- | | | 360] |
| | (D)Arg-(L)Ala-PAM-(D)Glu-(D)Lys- | | | |
| | (L)Phe-PAM-(D)Glu-(D)Arg-(L)Leu- | | | |
| | PAM-(D)Ala-(D)Arg-(L)Leu-PAM- | | | |
| | (D)Pro-(D)Arg-(L)Ala-PAM-(D)Tyr- | | | |
| | (D)Arg-(L)Phe-PAM | | | |
| IL6-65-11 | Fluorescein-(D)Tyr-(D)Leu-(L)Leu- | NNP2 | IL-6 | 25 |
| | PAM-Gly-(D)Arg-(L)Leu-PAM- | | | [6- 100] |
| | (D)Arg-(D)Ser-(L)Phe-PAM-(D)Pro- | | | |
| | (D)Arg-(L)Val-PAM-(D)Trp-(D)Arg- | | | |
| | (L)Val-PAM-(D)Asn-(D)Arg-(L)Phe- | | | |
| | PAM-(D)Glu-(D)Tyr-(L)Val-PAM- | | | |
| | (D)Pro-(D)Arg-(L)Ala-PAM-(D)Tyr- | | | |
| | (D)Arg-(L)Phe-PAM | | | |
| IL6-65-12 | Fluorescein-(D)Thr-(D)Leu-(L)Phe- | NNP2 | IL-6 | 80 |
| | PAM-Gly-(D)Arg-(L)Leu-PAM- | | | [40- 150] |
| | (D)Arg-(D)Ser-(L)Phe-PAM-(D)Pro- | | | |
| | (D)Arg-(L)Val-PAM-(D)Trp-(D)Arg- | | | |
| | (L)Val-PAM-(D)Asn-(D)Arg-(L)Phe- | | | |
| | PAM-(D)Glu-(D)Tyr-(L)Val-PAM- | | | |
| | (D)Pro-(D)Arg-Ala-PAM-Tyr-Arg- | | | |
| | Phe-PAM | | | |
| IL6R-87-1 | Fluorescein-(D)Arg-(D)Trp-(L)Phe- | NNP2 | SIL-6R | 1.2 |
| | PAM-(D)Pro-(D)Asn-(L)Val-PAM- | | | [0.40 - |
| | (D)Asn-(D)Thr-(L)Leu-PAM-(D)Lys- | | | 3.0] |
| | (D)Glu-(L)Leu-PAM-(D)Glu-(D)Lys- | | | |
| | (L)Phe-PAM-(D)Glu-(D)Trp-(L)Ala- | | | |
| | PAM-(D)Ala-(D)Arg-(L)Leu-PAM- | | | |
| | (D)Glu-(D)Arg-(L)Val-PAM-(D)Phe- | | | |
| | (D)Lys-Gly-PAM | | | |
| IL6R-87-2 | Fluorescein-(D)Tyr-(D)Leu-(L)Leu- | NNP2 | SIL-6R | 0.80 |
| | PAM-(D)Glu-(D)Ser-(L)Phe-PAM- | | | [0.14 - |
| | (D)Asn-(D)Thr-(L)Leu-PAM-(D)Lys- | | | 1.4] |
| | (D)Glu-(L)Leu-PAM-(D)Glu-(D)Lys- | | | |
| | (L)Phe-PAM-(D)Glu-(D)Arg-(L)Leu- | | | |
| | PAM-(D)Ala-(D)Arg-(L)Leu-PAM- | | | |
| | (D)Trp-(D)Lys-(L)Leu-PAM-(D)Phe- | | | |
| | (D)Lys-Gly-PAM | | | |
| IL6R-87-3 | Fluorescein-(D)Arg-(D)Trp-(L)Phe- | NNP2 | SIL-6R | 19 |
| | PAM-(D)Glu-(D)Ser-(L)Phe-PAM- | | | [11 - 34] |
| | (D)Asn-(D)Thr-(L)Leu-PAM-(D)Tyr- | | | |
| | (D)Asn-(L)Phe-PAM-(D)Tyr-(D)Lys- | | | |
| | (L)Ala-PAM-(D)Glu-(D)Arg-(L)Leu- | | | |
| | PAM-(D)Ala-(D)Arg-(L)Leu-PAM- | | | |
| | (D)Glu-(D)Arg-(L)Val-PAM-(D)Phe- | | | |
| | (D)Lys-Gly-PAM | | | |
| IL6R-87-4 | Fluorescein-(D)Asn-(D)Val-(L)Phe- | NNP2 | SIL-6R | 22 |
| | PAM-(D)Glu-(D)Ser-(L)Phe-PAM- | | | [8.1 - 35] |
| | (D)Arg-(D)Ser-(L)Phe-PAM-(D)Lys- | | | |
| | (D)Glu-(L)Leu-PAM-(D)Trp-(D)Arg- | | | |
| | (L)Val-PAM-(D)Pro-(D)Glu-(L)Phe- | | | |
| | PAM-(D)Ala-(D)Arg-(L)Leu-PAM- | | | |
| | (D)Pro-(D)Arg-(L)Ala-PAM-(D)Phe- | | | |
| | (D)Arg-Gly-PAM | | | |
| IL6R-87-5 | Fluorescein-(D)Arg-(D)Trp-(L)Phe- | NNP2 | SIL-6R | 330 |
| | PAM-(D)Leu-(D)Arg-(L)Leu-PAM- | | | [170 - |
| | (D)Asn-(D)Thr-(L)Leu-PAM-(D)Pro- | | | 770] |
| | (D)Arg-(L)Val-PAM-Gly-(D)Ala- | | | |
| | (L)Val-PAM-(D)Glu-(D)Arg-(L)Leu- | | | |
| | PAM-(D)Ala-(D)Arg-(L)Leu-PAM- | | | |
| | (D)Pro-(D)Arg-(L)Ala-PAM-(D)Phe- | | | |
| | (D)Arg-Gly-PAM | | | |
| IL6R-87-6 | Fluorescein-(D)Arg-(D)Trp-(L)Phe- | NNP2 | SIL-6R | 3.3 |
| | PAM-(D)Pro-(D)Asn-(L)Val-PAM- | | | [1.6- 6.8] |
| | (D)Pro-(D)Lys-(L)Ala-PAM-(D)Trp- | | | |
| | (D)Arg-(L)Leu-PAM-(D)Ala-(D)Lys- | | | |
| | (L)Phe-PAM-(D)Pro-(D)Glu-(L)Phe- | | | |
| | PAM-(D)Ala-(D)Arg-(L)Leu-PAM- | | | |
| | (D)Trp-(D)Lys-(L)Leu-PAM-(D)Phe- | | | |
| | (D)Lys-Gly-PAM | | | |
| IL6R-87-8 | Fluorescein-(D)Asn-(D)Val-(L)Phe- | NNP2 | SIL-6R | 0.62 |
| | PAM-(D)Tyr-(D)Ser-(L)Val-PAM- | | | [0.34 - |
| | (D)Arg-(D)Ser-(L)Phe-PAM-(D)Phe- | | | 1.1] |
| | (D)Arg-(L)Ala-PAM-(D)Tyr-(D)Lys- | | | |
| | (L)Ala-PAM-(D)Glu-(D)Arg-(L)Leu- | | | |
| | PAM-(D)Ala-(D)Arg-(L)Leu-PAM- | | | |
| | (D)Pro-(D)Arg-(L)Ala-PAM-(D)Phe- | | | |
| | (D)Lys-Gly-PAM | | | |
| IL6R-87-10 | Fluorescein-(D)Arg-(D)Trp-(L)Phe- | NNP2 | SIL-6R | 20 |
| | PAM-(D)Leu-(D)Arg-(L)Leu-PAM- | | | [5.4 - 67] |
| | (D)Phe-(D)Glu-(L)Val-PAM-(D)Lys- | | | |
| | (D)Glu-(L)Leu-PAM-(D)Ala-(D)Lys- | | | |
| | (L)Phe-PAM-(D)Asn-(D)Arg-(L)Phe- | | | |
| | PAM-(D)Ala-(D)Arg-(L)Leu-PAM- | | | |
| | (D)Trp-(D)Lys-(L)Leu-PAM-(D)Ala- | | | |
| | (D)Val-(L)Leu-PAM | | | |
| TNF-31-2 | Fluorescein-(D)Lys-(D)Val-(L)Leu- | NNP2 | TNFα | 23 |
| | PAM-(D)Ala-(D)Ala-(L)Phe-PAM- | | | [15-31] |
| | (D)Leu-(D)Lys-(L)Leu-PAM-(D)Phe- | | | |
| | (D)Lys-(L)Leu-PAM-(D)Trp-(D)Arg- | | | |
| | (L)Val-PAM-(D)Ser-(D)Ser-(L)Phe- | | | |
| | PAM-(D)His-(D)Glu-(L)Leu-PAM- | | | |
| | (D)Phe-(D)Lys-(L)Phe-PAM-(D)Thr- | | | |
| | (D)Glu-(L)Leu-PAM | | | |
| TNF-31-4 | Fluorescein-(D)Thr-(D)Leu-(L)Phe- | NNP2 | TNFα | 33 |
| | PAM-(D)Leu-(D)Arg-(L)Leu-PAM- | | | [22 - 50] |
| | (D)Leu-(D)Lys-(L)Leu-PAM-(D)Trp- | | | |
| | (D)Arg-(L)Leu-PAM-(D)Ala-(D)Lys- | | | |
| | (L)Phe-PAM-(D)Glu-(D)Ser-(L)Val- | | | |
| | PAM-(D)Pro-(D)Val-Gly-PAM- | | | |
| | (D)Glu-(D)Tyr-(L)Phe-PAM-(D)Tyr- | | | |
| | (D)Arg-(L)Phe-PAM | | | |
| TNF-31-5 | Fluorescein-(D)Lys-(D)Val-(L)Leu- | NNP2 | TNFα | 24 |
| | PAM-Gly-(D)Arg-(L)Leu-PAM- | | | [10-38] |
| | (D)Phe-(D)Glu-(L)Val-PAM-(D)Pro- | | | |
| | (D)Arg-(L)Val-PAM-(D)Ala-(D)Lys- | | | |
| | (L)Phe-PAM-(D)Glu-(D)Ser-(L)Val- | | | |
| | PAM-(D)Pro-(D) Val-Gly-PAM- | | | |
| | (D)Thr-(D)His-(L)Leu-PAM-(D)Tyr- | | | |
| | (D)Arg-(L)Phe-PAM | | | |
| TNF-31-7 | Fluorescein-(D)Lys-(D)Val-(L)Leu- | NNP2 | TNFα | 0.31 |
| | PAM-(D)Leu-(D)Arg-(L)Leu-PAM- | | | [0.080 - |
| | (D)Phe-(D)Glu-(L)Val-PAM-(D)Phe- | | | 1.2] |
| | (D)Arg-(L)Ala-PAM-(D)Ala-(D)Lys- | | | |
| | (L)Phe-PAM-(D)Phe-(D)Thr-(L)Ala- | | | |
| | PAM-(D)Glu-(D)Tyr-(L)Val-PAM- | | | |
| | Gly-(D)Glu-(L)Leu-PAM-(D)Trp- | | | |
| | (D)Glu-(L)Val-PAM | | | |
| TNF-31-10 | fluorescein-(D)Lys-(D)Val-(L)Leu- | NNP2 | TNFα | 53 |
| | PAM-(D)Leu-(D)Arg-(L)Leu-PAM- | | | [38 - 68] |
| | (D)Phe-(D)Glu-(L)Val-PAM-(D)Trp- | | | |
| | (D)Arg-(L)Leu-PAM-(D)Ala-(D)Lys- | | | |
| | (L)Phe-PAM-(D)Arg-(D)Tyr-(L)Leu- | | | |
| | PAM-(D)Pro-(D) Val-Gly-PAM- | | | |
| | (D)Lys-(D)Phe-(L)Phe-PAM-(D)His- | | | |
| | (D)Tyr-(L)Val-PAM | | | |
| TNF-31-14 | Fluorescein-(D)Tyr-(D)Leu-(L)Leu- | NNP2 | TNFα | 35 |
| | PAM-(D)Leu-(D)Arg-(L)Leu-PAM- | | | [17 - 49] |
| | (D)Leu-(D)Lys-(L)Leu-PAM-(D)Phe- | | | |
| | (D)Arg-(L)Ala-PAM-(D)Ala-(D)Lys- | | | |
| | (L)Phe-PAM-(D)Asn-(D)Arg-(L)Phe- | | | |
| | PAM-(D)Ala-(D)Arg-(L)Leu-PAM- | | | |
| | (D)Trp-(D)Lys-(L)Leu-PAM-(D)Pro- | | | |
| | (D)Ala-(L)Ala-PAM | | | |
| TNF-31-15 | Fluorescein-(D)Lys-(D)Val-(L)Leu- | NNP2 | TNFα | 23 |
| | PAM-(D)Leu-(D)Arg-(L)Leu-PAM- | | | [10- 36] |
| | (D)Arg-(D)Val-(L)Leu-PAM-(D)Phe- | | | |
| | (D)Arg-(L)Ala-PAM-(D)Tyr-(D)Lys- | | | |
| | (L)Ala-PAM-(D)Glu-(D)Arg-(L)Leu- | | | |
| | PAM-(D)Pro-(D)Val-Gly-PAM- | | | |
| | (D)Lys-(D)Phe-(L)Phe-PAM-(D)His- | | | |
| | (D)Glu-(L)Val-PAM | | | |
| TNF-31-16 | Fluorescein-(D)Lys-(D)Val-(L)Leu- | NNP2 | TNFα | 180 |
| | PAM-(D)Leu-(D)Arg-(L)Leu-PAM- | | | [80- 280] |
| | (D)Phe-(D)Glu-(L)Val-PAM-(D)Phe- | | | |
| | (D)Lys-(L)Leu-PAM-(D)Pro-(D)Glu- | | | |
| | (L)Leu-PAM-(D)Phe-(D)Thr-(L)Ala- | | | |
| | PAM-(D)His-(D)Glu-(L)Leu-PAM- | | | |
| | (D)Glu-(D)Tyr-(L)Phe-PAM-(D)Thr- | | | |
| | (D)Ser-(L)Val-PAM | | | |
| TNF-39-1 | Fluorescein-(D)Lys-(D)Val-(L)Leu- | NNP2 | TNFα | 116 |
| | PAM-(D)Leu-(D)Arg-(L)Leu-PAM- | | | [79 - 150] |
| | (D)Arg-(D)Val-(L)Leu-PAM-(D)Phe- | | | |
| | (D)Arg-(L)Ala-PAM-(D)Ala-(D)Lys- | | | |
| | (L)Phe-PAM-(D)Asn-(D)Arg-(L)Phe- | | | |
| | PAM-(D)Pro-(D)Val-Gly-PAM- | | | |
| | (D)Glu-(D)Arg-(L)Val-PAM-(D)Tyr- | | | |
| | (D)Arg-(L)Phe-PAM | | | |
| TNF-39-2 | Fluorescein-(D)Tyr-(D)Leu-(L)Leu- | NNP2 | TNFα | 200 |
| | PAM-Gly-(D)Arg-(L)Leu-PAM- | | | [150 - |
| | (D)Phe-(D)Glu-(L)Val-PAM-(D)Trp- | | | 250] |
| | (D)Arg-(L)Leu-PAM-(D)Trp-(D)Arg- | | | |
| | (L)Val-PAM-(D)Arg-(D)Tyr-(L)Leu- | | | |
| | PAM-(D)Pro-Gly-(L)Val-PAM-(D)His- | | | |
| | (D)Ser-(L)Leu-PAM-(D)Phe-(D)Lys- | | | |
| | Gly-PAM | | | |
| TNF-39-3 | Fluorescein-(D)Thr-(D)Leu-(L)Phe- | NNP2 | TNFα | 130 |
| | PAM-(D)Leu-(D)Arg-(L)Leu-PAM- | | | [92 - 170] |
| | (D)Phe-(D)Glu-(L)Val-PAM-(D)Phe- | | | |
| | (D)Arg-(L)Ala-PAM-(D)Ala-(D)Lys- | | | |
| | (L)Phe-PAM-(D)Arg-(D)Tyr-(L)Leu- | | | |
| | PAM-(D)Pro-Gly-(L)Val-PAM- | | | |
| | (D)Trp-(D)Lys-(L)Leu-PAM-(D)Tyr- | | | |
| | (D)Arg-(L)Phe-PAM | | | |
| TNF-39-4 | Fluorescein-(D)Thr-(D)Leu-(L)Phe- | NNP2 | TNFα | 28 |
| | PAM-(D)Leu-(D)Arg-(L)Leu-PAM- | | | [11 - 45] |
| | (D)Phe-(D)Glu-(L)Val-PAM-(D)Phe- | | | |
| | (D)Lys-(L)Leu-PAM-(D)Pro-(D)Glu- | | | |
| | (L)Leu-PAM-(D)Pro-(D)Glu-(L)Phe- | | | |
| | PAM-(D)His-(D)Glu-(L)Leu-PAM- | | | |
| | (D)Pro-(D)Arg-(L)Ala-PAM-(D)His- | | | |
| | (D)Tyr-(L)Val-PAM | | | |
| TNF-39-5 | Fluorescein-(D)Lys-(D)Val-(L)Leu- | NNP2 | TNFα | 23 |
| | PAM-(D)Leu-(D)Arg-(L)Leu-PAM- | | | [4 - 42] |
| | (D)Phe-(D)Glu-(L)Val-PAM-(D)Phe- | | | |
| | (D)Arg-(L)Ala-PAM-(D)Tyr-(D)Lys- | | | |
| | (L)Ala-PAM-(D)Glu-(D)Ser-(L)Val- | | | |
| | PAM-(D)Pro-(D)Val-Gly-PAM- | | | |
| | (D)Pro-(D)Arg-(L)Ala-PAM-(D)Phe- | | | |
| | (D)Lys-Gly-PAM | | | |
| TNF-39-6 | Fluorescein-(D)Tyr-(D)Leu-(L)Leu- | NNP2 | TNFα | 93 |
| | PAM-(D)Leu-(D)Arg-(L)Leu-PAM- | | | [87 - 96] |
| | (D)Phe-(D)Glu-(L)Val-PAM-(D)Pro- | | | |
| | (D)Arg-(L)Val-PAM-(D)Trp-(D)Arg- | | | |
| | (L)Val-PAM-(D)Arg-(D)Tyr-(L)Leu- | | | |
| | PAM-(D)Glu-(D)Val-(L)Val-PAM- | | | |
| | (D)Thr-(D)His-(L)Leu-PAM-(D)Tyr- | | | |
| | (D)Arg-(L)Phe-PAM | | | |
| TNF-39-7 | Fluorescein-(D)Lys-(D)Val-(L)Leu- | NNP2 | TNFα | 53 |
| | PAM-(D)Leu-(D)Arg-(L)Leu-PAM- | | | [6-100] |
| | (D)Arg-(D)Val-(L)Leu-PAM-(D)Phe- | | | |
| | (D)Lys-(L)Leu-PAM-(D)Ala-(D)Lys- | | | |
| | (L)Phe-PAM-(D)Phe-(D)Thr-(L)Ala- | | | |
| | PAM-(D)Pro-Gly-(L)Val-PAM- | | | |
| | (D)Lys-(D)Phe-(L)Phe-PAM-(D)Ala- | | | |
| | (D)Val-(L)Leu-PAM | | | |
| TNF-39-8 | Fluorescein-(D)Lys-(D)Val-(L)Leu- | NNP2 | TNFα | 270 |
| | PAM-(D)Trp-(D)His-(L)Ala-PAM- | | | [140 - |
| | (D)Phe-(D)Glu-(L)Val-PAM-(D)Trp- | | | 400] |
| | (D)Arg-(L)Leu-PAM-(D)Tyr-(D)Lys- | | | |
| | (L)Ala-PAM-(D)Asn-(D)Arg-(L)Phe- | | | |
| | PAM-(D)Pro-(D)Val-Gly-PAM- | | | |
| | (D)Pro-(D)Arg-(L)Ala-PAM-(D)Ala- | | | |
| | (D)Lys-Gly-PAM | | | |
| TNF-39-9 | Fluorescein-(D)Lys-(D)Val-(L)Leu- | NNP2 | TNFα | 150 |
| | PAM-Gly-(D)Arg-(L)Leu-PAM- | | | [120 - |
| | (D)Arg-(D)Val-(L)Leu-PAM-(D)Trp- | | | 180] |
| | (D)Arg-(L)Leu-PAM-(D)Trp-(D)Arg- | | | |
| | (L)Val-PAM-(D)Ser-(D)Ser-(L)Phe- | | | |
| | PAM-(D)Ala-(D)Ser-(L)Phe-PAM- | | | |
| | (D)Trp-(D)Lys-(L)Leu-PAM-(D)Tyr- | | | |
| | (D)Arg-(L)Phe-PAM | | | |
| TNF-39-10 | Fluorescein-(D)Lys-(D)Val-(L)Leu- | NNP2 | TNFα | 38 |
| | PAM-(D)Ala-(D)Ala-(L)Phe-PAM- | | | [19 - 57] |
| | (D)Phe-(D)Glu-(L)Val-PAM-(D)Pro- | | | |
| | (D)Arg-(L)Val-PAM-(D)Ala-Gly- | | | |
| | (L)Phe-PAM-(D)Glu-(D)Arg-(L)Leu- | | | |
| | PAM-(D)Pro-(D)Val-Gly-PAM- | | | |
| | (D)Trp-(D)Lys-(L)Leu-PAM-(D)Pro- | | | |
| | (D)Ala-(L)Ala-PAM | | | |
| TNF-39-11 | Fluorescein-(D)Lys-(D)Val-(L)Leu- | NNP2 | TNFα | 86 |
| | PAM-(D)Leu-(D)Arg-(L)Leu-PAM- | | | [52- 120] |
| | (D)Phe-(D)Glu-(L)Val-PAM-(D)Phe- | | | |
| | (D)Arg-(L)Ala-PAM-(D)Ala-(D)Lys- | | | |
| | (L)Phe-PAM-(D)Asn-(D)Arg-(L)Phe- | | | |
| | PAM-(D)Pro-(D)Val-Gly-PAM- | | | |
| | (D)Trp-(D)Lys-(L)Leu-PAM-(D)Tyr- | | | |
| | (D)Arg-(L)Phe-PAM | | | |
| TNF-39-12 | Fluorescein-(D)Lys-(D)Val-(L)Leu- | NNP2 | TNFα | 260 |
| | PAM-Gly-(D)Arg-(L)Leu-PAM- | | | [250 - |
| | (D)Leu-(D)Lys-(L)Leu-PAM-(D)Pro- | | | 270] |
| | (D)Arg-(L)Val-PAM-(D)Ala-(D)Lys- | | | |
| | (L)Phe-PAM-(D)Glu-(D)Trp-(L)Ala- | | | |
| | PAM-(D)His-(D)Glu-(L)Leu-PAM- | | | |
| | (D)Glu-(D)Arg-(L)Val-PAM-(D)Thr- | | | |
| | (D)Ser-(L)Val-PAM | | | |
| TNF-39-13 | Fluorescein-(D)Ser-(D)Val-(L)Leu- | NNP2 | TNFα | 40 |
| | PAM-Gly-(D)Arg-(L)Leu-PAM- | | | [17 - 63] |
| | (D)Phe-(D)Glu-(L)Val-PAM-(D)Phe- | | | |
| | (D)Lys-(L)Leu-PAM-(D)Tyr-(D)Lys- | | | |
| | (L)Ala-PAM-(D)Glu-(D)Trp-(L)Ala- | | | |
| | PAM-(D)Ala-(D)Arg-(L)Leu-PAM- | | | |
| | (D)Glu-(D)Tyr-(L)Phe-PAM-(D)Tyr- | | | |
| | (D)Arg-(L)Phe-PAM | | | |
| TNF-39-14 | Fluorescein-(D)Tyr-(D)Leu-(L)Leu- | NNP2 | TNFα | 36 |
| | PAM-(D)Leu-(D)Arg-(L)Leu-PAM- | | | [14 - 50] |
| | (D)Leu-(D)Lys-(L)Leu-PAM-(D)Phe- | | | |
| | (D)Arg-(L)Ala-PAM-(D)Ala-(D)Lys- | | | |
| | (L)Phe-PAM-(D)Phe-(D)Thr-(L)Ala- | | | |
| | PAM-(D)Pro-(D)Val-Gly-PAM- | | | |
| | (D)Thr-(D)His-(L)Leu-PAM-(D)Thr- | | | |
| | (D)Ser-(L)Val-PAM | | | |
| TNF-39-15 | Fluorescein-(D)Lys-(D)Val-(L)Leu- | NNP2 | TNFα | 46 |
| | PAM-Gly-(D)Arg-(L)Leu-PAM- | | | [22 - 54] |
| | (D)Phe-(D)Glu-(L)Val-PAM-(D)Phe- | | | |
| | (D)Arg-(L)Ala-PAM-(D)Trp-(D)Arg- | | | |
| | (L)Val-PAM-(D)Glu-(D)Arg-(L)Leu- | | | |
| | PAM-(D)His-(D)Glu-(L)Leu-PAM- | | | |
| | (D)Lys-(D)Phe-(L)Phe-PAM-(D)Tyr- | | | |
| | (D)Arg-(L)Phe-PAM | | | |

Table 14 shows a hit summary for the five targets, including the hit process rate for each target at each stage of hit confirmation. For NNP1 screens where multiple copies of the library were screened hit redundancy was significant and therefore hit sequences were clustered to simplify reconfirmation.

TABLE 14

| | | | # Hits | | | | # of Hits |
|---|---|---|---|---|---|---|---|
| | | | after | | | | reconfir. |
| | | Est. # of | Screen | # of | | | for |
| | | Compounds | and | Isolated | # of Hits | # of Hits | binding |
| Target | Library | Screened | Confirm. | Hits | Sequenced | Synthesized | by MST |
| K-Ras | NNP1 | 5,000,000 | 381 | 105 | 85 | 14 (one | 14 |
| | | (2 plates, | | | | of each | |
| | | 20 um beads) | | | | cluster) | |
| ASGPR | NNP1 | 5,000,000 | 289 | 193 | 190 | 19 (one | 8 |
| | | (2 plates, | | | | of each | |
| | | 20 um beads) | | | | cluster) | |
| IL-6 | NNP2 | 10,000,000 | 19 | 19 | 14 | 12 | 8 |
| | | (2 plates, | | | | | |
| | | 10 um beads) | | | | | |
| IL-6R | NNP2 | 10,000,000 | 120 | 82 | 46 | 13 (one | 8 |
| | | (2 plates, | | | | of each | |
| | | 10 um beads) | | | | cluster) | |
| TNFα | NNP2 | 10,000,000 | 44 | 44 | 43 | 27 | 23 |
| | | (2 plates, | | | | | |
| | | 10 um beads) | | | | | |

FIGS. 13A-13B illustrate example distribution of amino acids in libraries, in accordance with the present disclosure.

FIG. 13A is a graph showing the order of the amino acids with the x axis being sorted from the least abundant (left) to the most (right) in the genome (based on data from the UCSC Proteome Browser). The blue bars represent the number of each amino acid in NNP1 library. Table 15 shows an amino acid distribution in the NNP1 library.

TABLE 15

| Amino | | | | | | |
|---|---|---|---|---|---|---|
| Acid | Ptych 6 | Ptych 5 | Ptych 4 | Ptych 3 | Ptych 2 | Ptych 1 |
| Trp | 2 | 2 | 2 | 2 | 2 | 2 |
| His | 5 | 1 | 3 | 2 | 3 | 3 |
| Tyr | 2 | 3 | 2 | 3 | 3 | 5 |
| Asn | 5 | 3 | 3 | 2 | 2 | 1 |
| Phe | 5 | 5 | 3 | 2 | 2 | 3 |
| Lys | 0 | 4 | 3 | 3 | 2 | 0 |
| Arg | 1 | 3 | 5 | 5 | 4 | 5 |
| Thr | 2 | 3 | 3 | 5 | 3 | 5 |
| Val | 4 | 4 | 4 | 4 | 5 | 3 |
| Pro | 8 | 4 | 5 | 4 | 6 | 6 |
| Gly | 2 | 5 | 5 | 6 | 5 | 4 |
| Glu | 6 | 4 | 4 | 5 | 6 | 4 |
| Ala | 4 | 4 | 4 | 4 | 3 | 4 |
| Ser | 4 | 4 | 5 | 3 | 5 | 6 |
| Leu | 5 | 6 | 4 | 5 | 4 | 4 |

FIG. 13B is a graph showing the order of the amino acids with the x axis being sorted from the least abundant (left) to the most (right) in the genome (based on data from the UCSC Proteome Browser). The blue bars represent the number of each amino acid in NNP1 library. Table 16 shows an amino acid distribution in the NNP1 library.

TABLE 16

| Amino | Ptych | Ptych | Ptych | Ptych | Ptych | Ptych | Ptych | Ptych | Ptych |
|---|---|---|---|---|---|---|---|---|---|
| Acid | 9 | 8 | 7 | 6 | 5 | 4 | 3 | 2 | 1 |
| Trp | 1 | 1 | 1 | 1 | 1 | 1 | 1 | 1 | 1 |
| His | 0 | 1 | 2 | 1 | 1 | 1 | 1 | 2 | 2 |
| Tyr | 2 | 3 | 1 | 1 | 1 | 2 | 1 | 1 | 2 |
| Asn | 1 | 1 | 1 | 1 | 1 | 1 | 1 | 0 | 0 |
| Phe | 6 | 2 | 4 | 4 | 3 | 5 | 2 | 3 | 3 |
| Lys | 1 | 1 | 2 | 2 | 4 | 1 | 2 | 2 | 1 |
| Arg | 2 | 4 | 2 | 3 | 1 | 3 | 2 | 2 | 2 |
| Thr | 2 | 1 | 1 | 1 | 1 | 1 | 1 | 1 | 2 |
| Val | 4 | 4 | 2 | 3 | 2 | 1 | 4 | 2 | 5 |
| Pro | 0 | 1 | 1 | 1 | 2 | 1 | 1 | 1 | 1 |
| Gly | 0 | 1 | 3 | 1 | 2 | 1 | 1 | 2 | 2 |
| Glu | 2 | 1 | 4 | 2 | 2 | 4 | 4 | 4 | 3 |
| Ala | 0 | 3 | 1 | 3 | 4 | 3 | 2 | 3 | 3 |
| Ser | 2 | 2 | 1 | 2 | 1 | 3 | 2 | 15 | 1 |
| Leu | 7 | 4 | 4 | 4 | 4 | 2 | 5 | 5 | 2 |

The following provides various materials and chemical synthesis information for the experimental embodiments described above.

Materials for Library and Peptide Synthesis.

Fmoc-L- and D-amino acids, Fmoc-4-aminomethyl-phenylacetic acid (fmoc-PAM), trifluoroacetic acid (TFA; ≥99%), 0-(7-Azabenzotriazol-1-yl)-N,N,N′,N′-tetramethyluronium hexafluorophosphate (HATU; ≥99%), ethyl (hydroxyimino)cyanoacetate (Oxyma; ≥99%), N-(9-Fluorenylmethoxycarbonyloxy)succinimide (fmoc-OSu), tetrahydrofuran (THF; anhydrous) and 1-Methyl-2-pyrrolidinone (NMP; for GC, distilled, ≥99.8%) were obtained from Chem-Impex International, Inc. (Wood Dale, Illinois). N,N-Diisopropylethylamine (DIEA; purified by redistillation, 99.5%), triisopropylsilane (98%) and N,N′-Diisopropylcarbodiimide (DIC; 99%) were purchased from MilliporeSigma (St. Louis, MO). N,N-dimethylformamide (DMF; for sequencing, Fisher BioReagents, ≥99.5%), dichloromethane (DCM), methanol (MeOH; HPLC grade), 4-methyl-piperidine (99%, ACROS Organics™) and NHS-Fluorescein (5/6-carboxyfluorescein succinimidyl ester, mixed isomer) were purchased from Fisher Scientific (Chicago, Illinois). Ethyl acetate (EtOAc) was obtained from VWR Chemicals BDH. Boc-L-Ala-OCH₂-phenylacetic acid (boc-L-Ala-PAM ester), boc-L-Phe-OCH₂-phenylacetic acid (boc-L-Phe-PAM ester), boc-L-Val-OCH₂-phenylacetic acid (boc-L-Val-PAM ester), boc-L-Leu-OCH₂-phenylacetic acid (boc-L-Leu-PAM ester) and boc-Gly-OCH₂-phenylacetic acid (boc-Gly-PAM ester) were purchased from PolyPeptide Laboratories (San Diego, California). 10 μm TentaGel M NH₂ monosized amino TentaGel® microspheres (M30102; 0.25 mmol/g amine loading) and 20 um TentaGel® M NH₂ monosized amino TentaGel microspheres (M30202; 0.27 mmol/g amine loading) were purchased from Rapp Polymere (Tubingen, Germany). H-Rink Amide-ChemMatrix® resin was purchased from Biotage (Charlotte, North Carolina). Amino acids used were fmoc-D-Ala-OH, fmoc-L-Ala-OH, fmoc-D-Arg(Pbf)-OH, fmoc-D-Asn(Trt)-OH, Fmoc-D-His(Trt)-OH, fmoc-Gly-OH, fmoc-D-Glu(tBu)-OH, fmoc-D-Leu-OH, fmoc-L-Leu-OH, fmoc-D-Lys(boc), fmoc-D-Phe-OH, fmoc-L-Phe-OH, fmoc-D-Pro-OH, fmoc-D-Ser-OH, fmoc-D-Thr(tBu)-OH, fmoc-D-Trp(boc)-OH, fmoc-D-Tyr(tBu), fmoc-D-Val-OH, fmoc-L-Val-OH. Other chemicals and reagents were purchased from MilliporeSigma (St. Louis, MO).

Materials for Screening, MST Analysis and Biological Assays.

Odyssey blocking buffer PBS was purchased from LI-COR Biosciences (Lincoln, Nebraska). FACS buffer was obtained from BD Biosciences (San Jose, California). Alexa Fluor 555—NHS ester (Succinimidyl Ester) Dye (AF555) was purchased from Fisher Scientific (Chicago, Illinois). CF555—NHS ester dye was obtained from Biotium, Inc (Fremont, California). Recombinant Human ASGR1/ASGPR1 asialoglycoprotein receptor (ASGPR; catalog number: 4394-AS) and Recombinant Human TNF-α (catalog number: 210-TA/CF) were purchased from R&D Systems, Inc. K-Ras4B G12V (K-Ras; catalog number: CS-RS04) was purchased from Cytoskeleton Inc. (Denver, Colorado). Raf-RBD (Raf; catalog number: PR-305) was purchased from Jena Bioscience (Jena, Germany). Recombinant human interleukin-6 protein (IL-6; catalog number: IL6-12H) and recombinant human interleukin-6 receptor (IL-6R; catalog number: IL6R-584H) were obtained from Creative Biomart (Shirley, New York). HEK293T and HepG2 cells were purchased from American Type Culture Collection (ATCC, Manassas, VA, USA). Asialofetuin (from fetal calf serum), human serum, mouse serum and Angiotensin I peptide were purchased from MilliporeSigma. GppNHp, non-hydrolyzable GTP analog (GTP; catalog number: ab146659) was purchased from Abcam (Cambridge, United Kingdom). The precursor N-acetylgalactosamine (GalNAc) ligand used in the synthesis of the reference trivalent GalNAc ligand for ASGPR cell uptake assays was kindly provided by Ionis Pharmaceuticals, Inc. (Carlsberg, California).

LC-MS Analysis.

Resynthesized hits (crude and purified hits) were evaluated by analytical reversed-phase (RP) LC-MS (Thermo Fisher Scientific LCQ Fleet with ion trap mass spectrometer) with the mobile phase of solvent A (0.1% formic acid in water) and solvent B (0.1% formic acid in acetonitrile) on a C18 column (Agilent Pursuit 3 C18, 2.1×50 mm, 3 μm particle size). The HPLC conditions used for analysis were 5% B from 0-1 minute, then 5% to 95% from 1-10 minutes at 0.5 mL/min flow rate.

Preparative LC-MS Purification.

Resynthesized hits were purified on a preparative LCMS (Shimadzu LCMS-2020 single quadrupole LCMS) using Agilent 300SB-C18 Semi-Prep HPLC Column 9.4×250 mm (10 μm particle size) with mobile phase of solvent A (0.1% TFA in water) and solvent B (0.1% TFA in 6:3:1 acetonitrile isopropanol:water). Unless stated otherwise, the LC conditions used for purification were 1% B from 0-3 minute, 1% to 57% from 3-14 minutes, then 57% to 62% from 14-29 minutes at 4 mL/min flow rate.

Analytical HPLC Analysis.

After purification, fractions containing the purified NNP were identified by LC, and aliquots of the selected fractions were combined and checked by LC-MS. Selected fractions were combined and lyophilized. Analytical HPLC was performed on Waters RP-HPLC (Waters Alliance 2695) using an XBridge C18 column (4.6 mm×250 mm, 5 μm particle size) and mobile phase of solvent A (0.1% TFA in water) and solvent B (0.1% TFA in acetonitrile). The LC conditions used for analysis were 5% B from 0-1 minute, then 5% to 65% from 1-16 minutes at 1 mL/min flow rate.

Purification of fmoc-amino-acid-PAM esters. Normal-phase column chromatography was performed with a Biotage Isolera One flash purification system equipped with 80 g Biotage ZIP Sphere columns with a gradient of MeOH/DCM as follows: 0% MeOH, 1 column volume (CV); 0-10% MeOH, 10 CV; 10% MeOH, 2 CV.

Synthesis.

Synthesis of fmoc-L-Ala-OCH₂-phenylacetic acid (fmoc-L-Ala-PAM ester) (3).

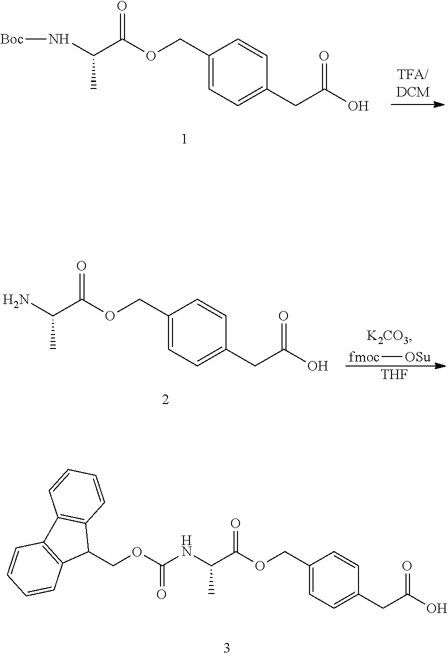

L-Ala-OCH₂-phenylacetic acid (L-Ala-PAM) (2): Boc-L-Ala-OCH₂-phenylacetic acid 1 (2 g, 5.92 mmol) was treated with a mixture of TFA and DCM (3 mL and 5 mL, respectively) and the reaction mixture was stirred overnight at room temperature. Upon completion (confirmed by TLC and LC-MS), the solvent was evaporated to yield crude amine 2, which was carried on to the next step without further purification.

Fmoc-L-Ala-OCH₂-phenylacetic acid (fmoc-L-Ala-PAM) (3): Aqueous potassium carbonate (15 mL, 2M, 29.6 mmol, 5 eq.) and 15 mL THF were added to crude amine 2 and the reaction mixture was cooled down in an ice bath for 10 minutes. Then, fmoc-OSu (2.2 g, 6.5 mmol, 1.1 eq.) was added stepwise, and the reaction mixture was stirred at 0 degrees C. for 30 minutes and then at room temperature for 1 hour, and was monitored by TLC and LC-MS. Upon completion, the reaction mixture was acidified with 10% w/w citric acid in water to pH<5 and was extracted twice with EtOAc. The organic layers were combined, washed with brine and dried with MgSO₄. Crude product 3 was purified using normal-phase column chromatography performed on a Biotage Isolera One flash purification system equipped with a 80 g Biotage ZIP Sphere column. The gradient used for purification was as follows: 0% MeOH, 1 column volume (CV); 0-10% MeOH, 10 CV; 10% MeOH, 2 CV, to yield 2.46 g (90% yield for the two steps) of fmoc-L-Ala-PAM ester 3. LC-MS (ESI): [M-(C₂₇H₂₅NO₆)+H]⁺ calculated: 460.03 Da; found: 459.4 Da. Fmoc-L-Phe-OCH₂-phenylacetic acid (fmoc-L-Phe-PAM ester), fmoc-L-Val-OCH₂-phenylacetic acid (fmoc-L-Val-PAM ester), fmoc-L-Leu-OCH₂-phenylacetic acid (fmoc-L-Leu-PAM ester) and fmoc-Gly-OCH₂-phenylacetic acid (fmoc-Gly-PAM ester) were synthesized in the same manner and used in the synthesis of the two libraries.

Synthesis of GalNAc probe as ASGPR reference ligand (THA-(GalNAc)₃-(OAc)₉-1,3-propanediamine-fluorescein 8):

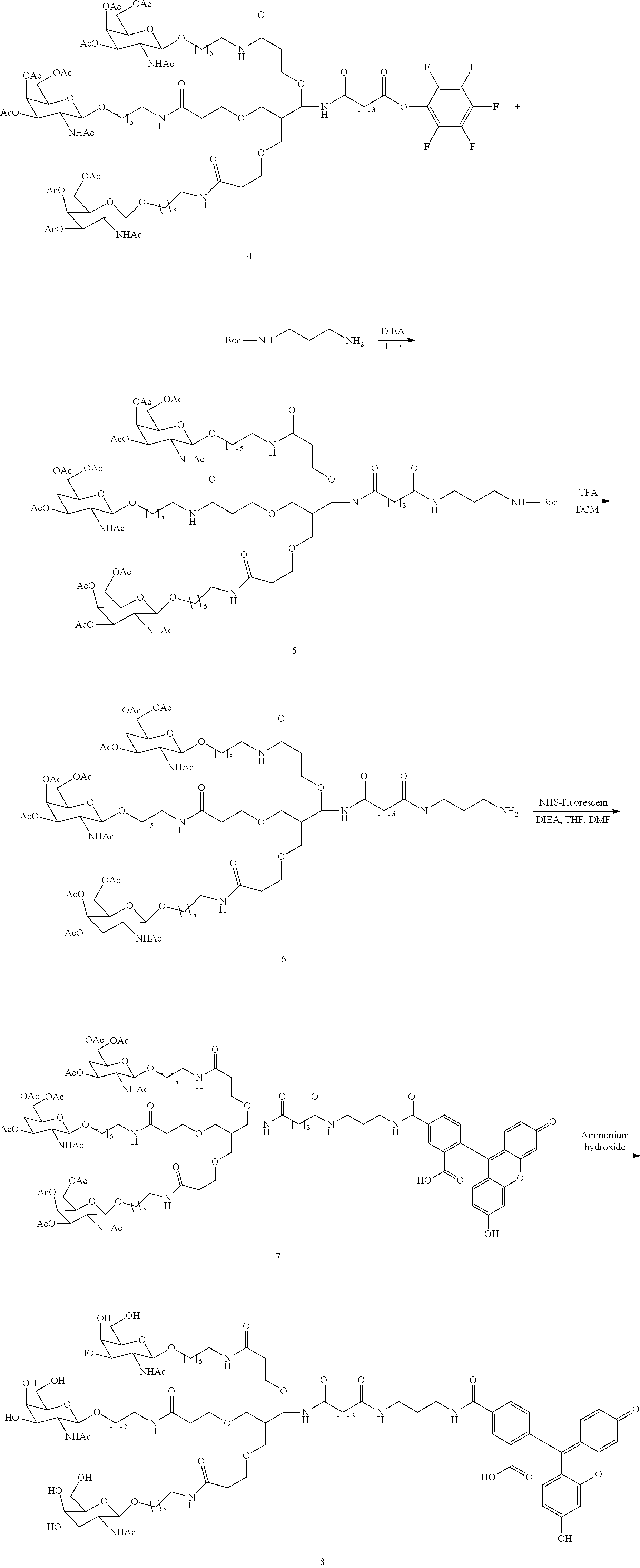

Trishexylamino-(GalNAc)₃-(OAc)₉-pentafluorophenyl ester (THA-(GalNAc)₃-(OAc)₉-PFP ester 4) was kindly provided by Ionis Pharmaceuticals, Inc.

THA-(GalNAc)₃-(OAc)₉—N-boc-1,3-propanediamine (5): To a solution of THA-(GalNAc)₃-(OAc)₉—PFP ester 4 (5 mg, 0.0026 mmol) in 200 μL THF, N-boc-1,3-propanediamine (1.1 eq., 0.0029 mmol, 0.5 μL) and DIEA (1.1 eq., 0.0029 mmol, 0.5 μL) were added and the reaction mixture was stirred at room temperature overnight. Upon completion (confirmed by LC-MS), the THF was evaporated, and the crude product 5 was carried on to the next step without further purification. LC-MS (ESI): [M-(C₈₆H₁₄₁N₉O₃₇)+H]⁺ calculated: 1893.1 Da; found: 1893.6 Da.

THA-(GalNAc)₃-(OAc)₉-1,3-propanediamine (6): A solution of 10% TFA in DCM (50 μL TFA+450 μL DCM) was added to the dried crude product 5, and the reaction mixture was stirred and monitored by LC-MS. Upon completion after 1 hour, the solvents were evaporated, and the reaction mixture was dried on high vacuum. Crude 6 was carried on to the next step without further purification. LC-MS (ESI): [M-(C₈₁H₁₃₃N₉O₃₅)+H]⁺ calculated: 1791.9 Da; found: 1792.1 Da.

THA-(GalNAc)₃-(OAc)₉-1,3-propanediamine-fluorescein (7): DIEA (20 eq., 0.053 mmol, 9.2 μL) and NHS-fluorescein (10 eq., 0.026 mmol, 12.3 mg) were added to a stirring solution of crude product 6 in 500 μL THF and 200 μL DMF. The reaction mixture was stirred at room temperature overnight and was confirmed for completion by LC-MS. The solvents were evaporated, and the reaction mixture was dried on high-vacuum. Crude 7 was carried on to the next step without further purification. LC-MS (ESI): [M-(C₁₀₂H₁₄₃N₉O₄₁)+H]⁺ calculated: 2151.3 Da; found: 2151.6 Da.

THA-(GalNAc)₃-(OH)₉-1,3-propanediamine-fluorescein (8): ammonium hydroxide (28-30% ammonia in water; 1 mL) was added to crude product 7 and the reaction mixture was stirred for 3 h at room temperature and monitored by LC-MS. Upon completion, the reaction mixture was evaporated and dried on the SpeedVac. Then the crude was dissolved in a solution of 1:1 DMSO:water and purified on a preparative LC-MS using Agilent 300SB-C8 Semi-Prep HPLC Column 9.4×250 (10 μm particle size) with mobile phase of solvent A (0.1% TFA in water) and solvent B (0.1% TFA in acetonitrile). The LC conditions used for purification were as follows: 5% B from 0-1 minute, then 5% to 60% from 1-56 minutes at 4 mL/minute flow rate. Fractions containing purified compound 8 were identified by LC, and aliquots of the selected fractions were combined and checked by LC-MS. Selected fractions were combined and lyophilized to yield pure THA-(GalNAc)₃-(OH)₉-1,3-propanediamine-fluorescein. LC-MS (ESI): [M-(C₈₄H₁₂₅N₉O₃₂)+H]⁺ calculated: 1771.8 Da; found: 1771.8 Da.

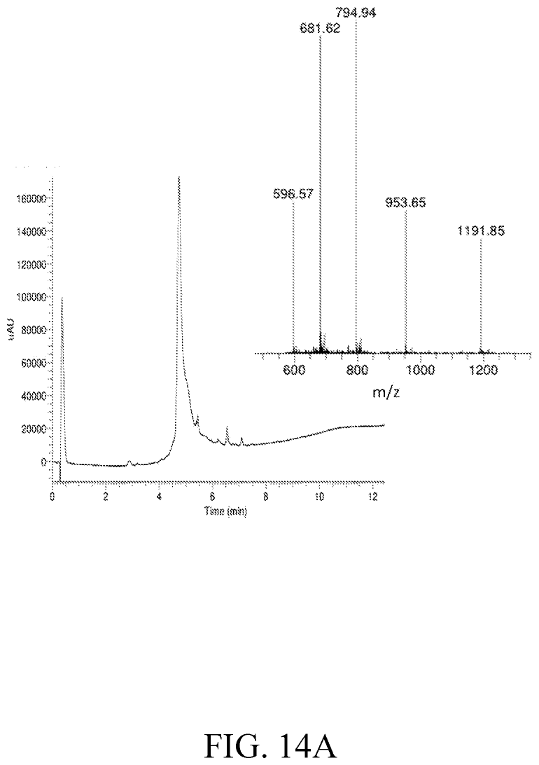

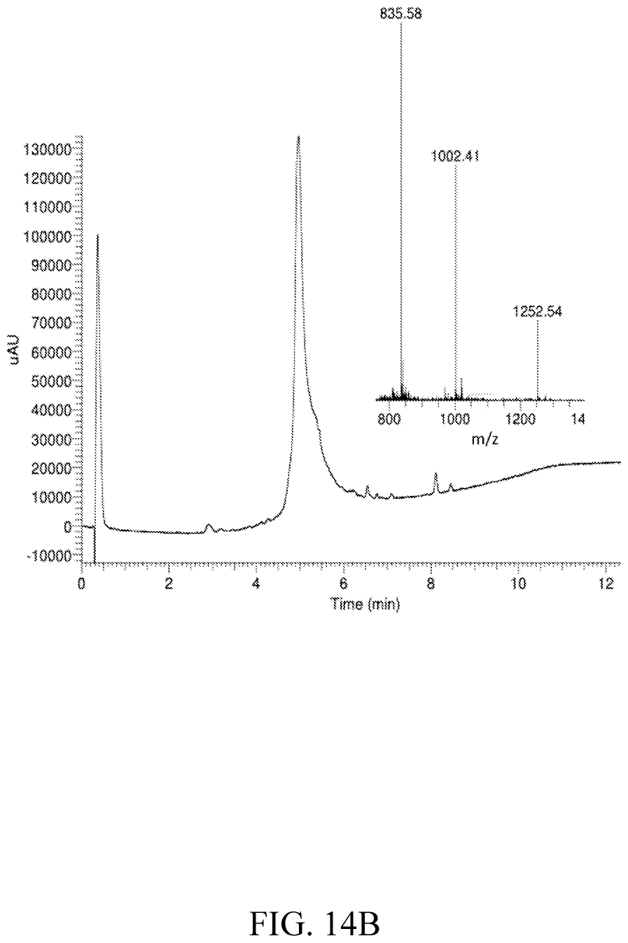

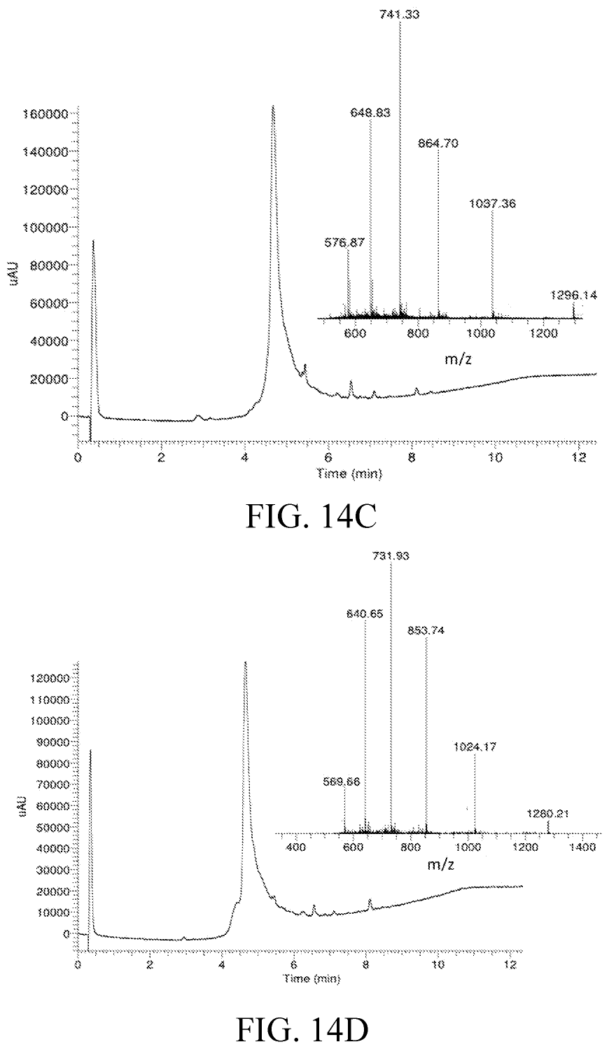

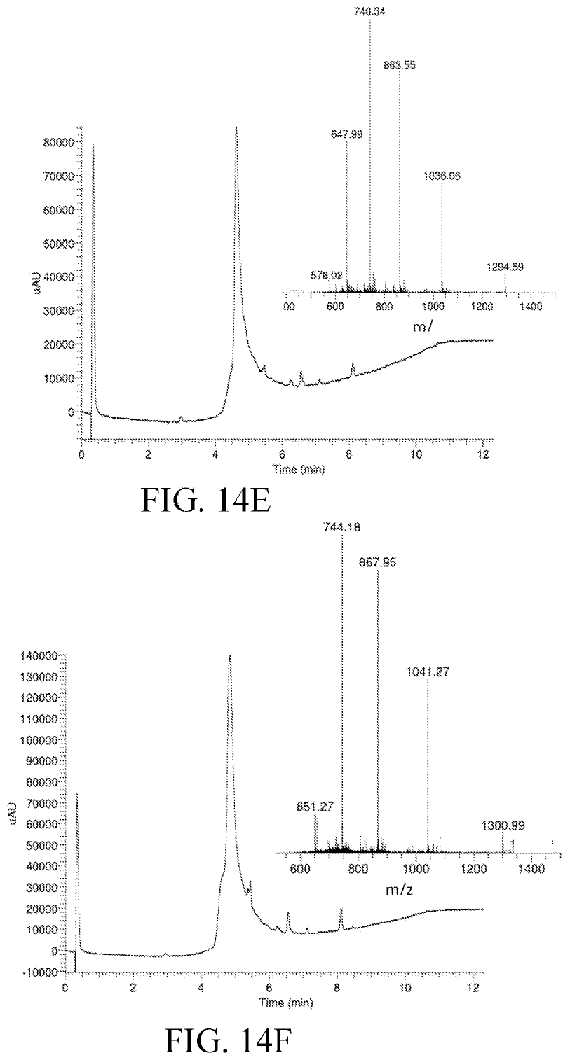

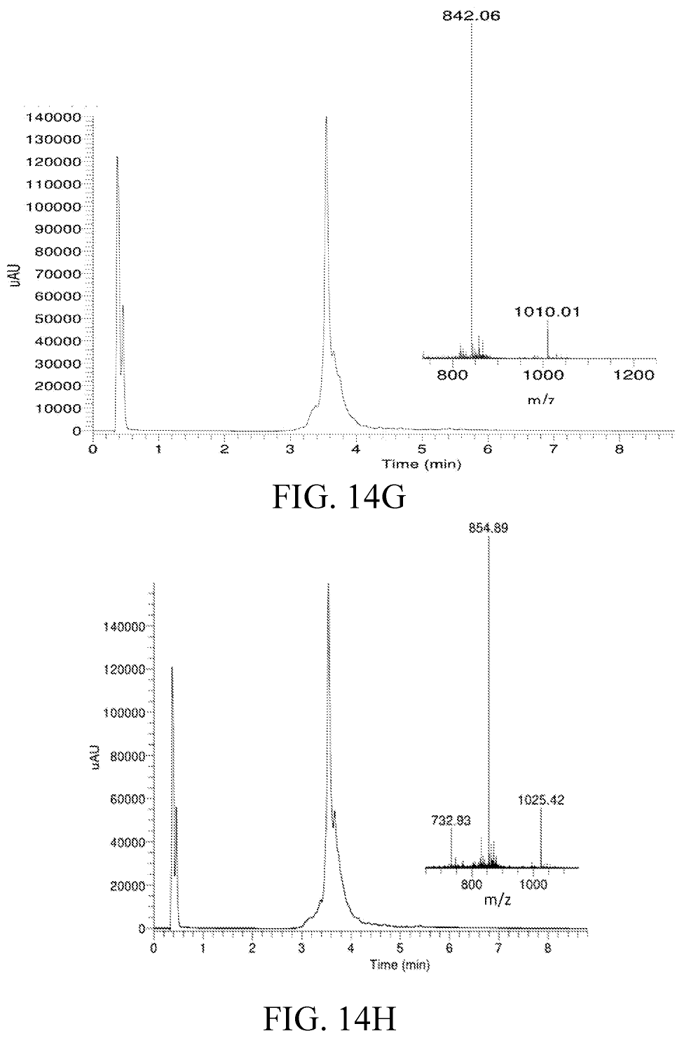

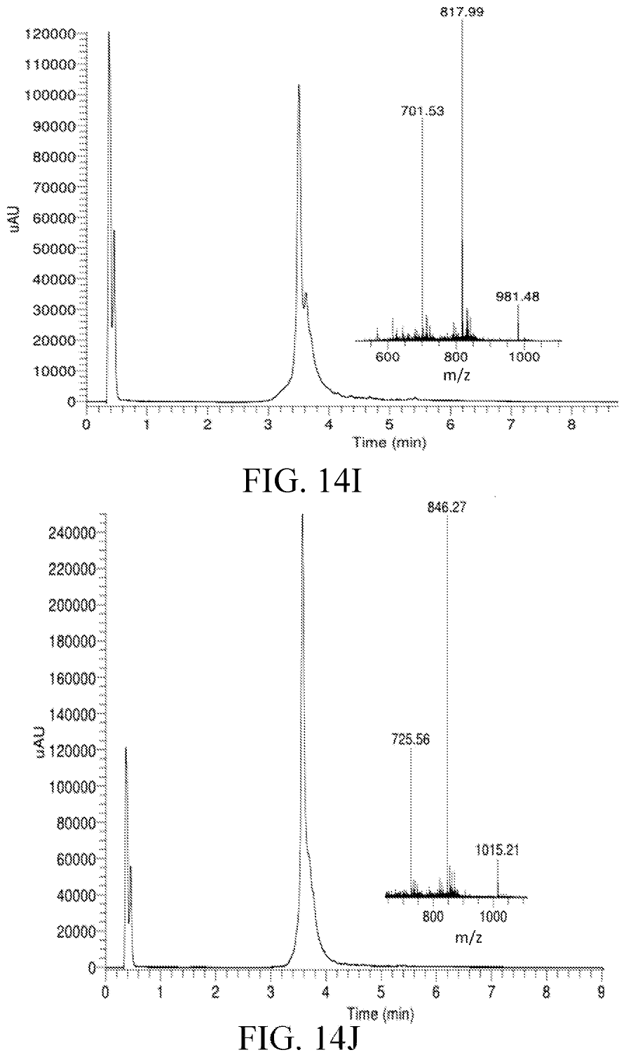

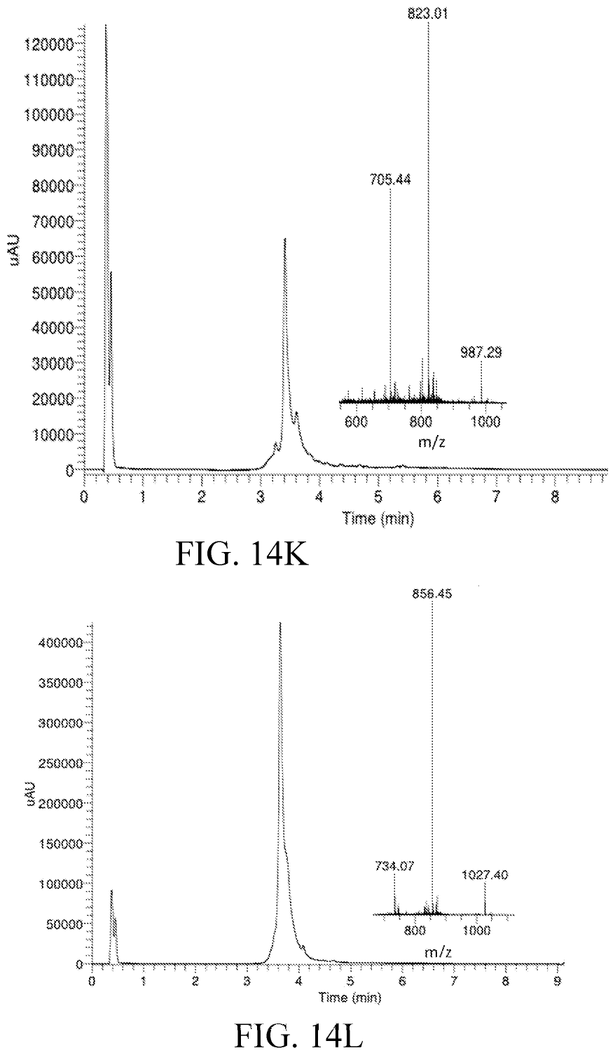

FIGS. 14A-14L illustrate example mass spectrometry results of hits from screening an assay, in accordance with the present disclosure.

FIG. 14A is a graph showing the LC-MS analysis of the synthetic compound IL6-65-2 with the sequence of Fluorescein-(D)His-(D)Leu-(L)Phe-PAM-(D)Ala-(D)Ala-(L)Phe-PAM-(D)Pro-(D)Lys-(L)Ala-PAM-(D)Phe-(D)Arg-(L)Ala-PAM-(D)Ala-(D)Lys-(L)Phe-PAM-(D)Phe-(D)Thr-(L)Ala-PAM-(D)Lys-(D)Asn- (L)Leu-PAM-(D)Thr-(D)His-(L)Leu-PAM-(D)Phe-(D)Lys-Gly-PAM. LC-MS (ESI): calculated: 4763.4 Da; found: 4763.8±0.6 Da.

FIG. 14B is a graph showing the LC-MS analysis of the synthetic compound IL6-65-3 with the sequence of Fluorescein-(D)Arg-(D)Trp-(L)Phe-PAM-(D)Glu-(D)Ser-(L)Phe-PAM-(D)Arg-(D)Ser-(L)Phe-PAM-(D)Trp-(D)Arg-(L)Leu-PAM-Gly-(D)Ala-(L)Val-PAM-(D)Asn-(D)Arg-(L)Phe-PAM-(D)Ala-(D)Ser-(L)Phe- PAM-(D)Pro-(D)Arg-(L)Ala-PAM-(D)Phe-(D)Lys-Gly-PAM. LC-MS (ESI): calculated: 5006.5 Da; found: 5006.9±0.7 Da.

FIG. 14C is a graph showing the LC-MS analysis of the synthetic compound IL6-65-4 with the sequence of Fluorescein-(D)Arg-(D)Trp-(L)Phe-PAM-(D)Leu-(D)Arg-(L)Leu-PAM-(D)His-(D)Tyr-(L)Phe-PAM-(D)Trp-(D)Arg-(L)Leu-PAM-(D)His-(D)Lys-(L)Leu-PAM-(D)Ala-(D)Tyr-Gly-PAM-(D)His-(D)Glu-(L)Leu- PAM-(D)Pro-(D)Arg-(L)Ala-PAM-(D)Phe-(D)Lys-Gly-PAM. LC-MS (ESI): calculated: 5181.9 Da; found: 5181.9±0.9 Da.

FIG. 14D is a graph showing the LC-MS analysis of the synthetic compound IL6-65-7 with the sequence of Fluorescein-(D)Tyr-(D)Leu-(L)Leu-PAM-Gly-(D)Arg-(L)Leu-PAM-(D)Arg-(D)Ser-(L)Phe-PAM-(D)Phe-(D)Arg-(L)Ala-PAM-(D)Glu-(D)Lys-(L)Phe-PAM-(D)Glu-(D)Arg-(L)Leu-PAM-(D)Ala-(D)Arg-(L)Leu- PAM-(D)Pro-(D)Arg-(L)Ala-PAM-(D)Tyr-(D)Arg-(L)Phe-PAM. LC-MS (ESI): calculated: 5116.8 Da; found: 5116.6±0.5 Da.

FIG. 14E is a graph showing the LC-MS analysis of the synthetic compound IL6-65-8 with the sequence of Fluorescein-(D)Tyr-(D)Glu-(L)Leu-PAM-(D)Leu-(D)Arg-(L)Leu-PAM-(D)Arg-(D)Ser-(L)Phe-PAM-(D)Phe-(D)Arg-(L)Ala-PAM-(D)Glu-(D)Lys-(L)Phe-PAM-(D)His-(D)Lys-(L)Phe-PAM-(D)Pro-Gly-(L)Val- PAM-(D)Glu-(D)Arg-(L)Val-PAM-(D)Tyr-(D)Arg-(L)Phe-PAM. LC-MS (ESI): calculated: 5174.8 Da; found: 5175.2±0.6 Da.

FIG. 14F is a graph showing the LC-MS analysis of the synthetic compound IL6-65-11 with the sequence of Fluorescein-(D)Tyr-(D)Leu-(L)Leu-PAM-Gly-(D)Arg-(L)Leu-PAM-(D)Arg-(D)Ser-(L)Phe-PAM-(D)Pro-(D)Arg-(L)Val-PAM-(D)Trp-(D)Arg-(L)Val-PAM-(D)Asn-(D)Arg-(L)Phe-PAM-(D)Glu-(D)Tyr-(L)Val- PAM-(D)Pro-(D)Arg-(L)Ala-PAM-(D)Tyr-(D)Arg-(L)Phe-PAM. LC-MS (ESI): calculated: 5201.9 Da; found: 5201.5±0.9 Da.

FIG. 14G is a graph showing the LC-MS analysis of the synthetic compound IL6R-87-1 with the sequence of Fluorescein-(D)Arg-(D)Trp-(L)Phe-PAM-(D)Pro-(D)Asn-(L)Val-PAM-(D)Asn-(D)Thr-(L)Leu-PAM-(D)Lys-(D)Glu-(L)Leu-PAM-(D)Glu-(D)Lys-(L)Phe-PAM-(D)Glu-(D)Trp-(L)Ala-PAM-(D)Ala-(D)Arg- (L)Leu-PAM-(D)Glu-(D)Arg-(L)Val-PAM-(D)Phe-(D)Lys-Gly-PAM. LC-MS (ESI): calculated: 5045.6 Da; found: 5045.7±0.9 Da.

FIG. 14H is a graph showing the LC-MS analysis of the synthetic compound IL6R-87-3 with the sequence of Fluorescein-(D)Arg-(D)Trp-(L)Phe-PAM-(D)Glu-(D)Ser-(L)Phe-PAM-(D)Asn-(D)Thr-(L)Leu-PAM-(D)Tyr-(D)Asn-(L)Phe-PAM-(D)Tyr-(D)Lys-(L)Ala-PAM-(D)Glu-(D)Arg-(L)Leu-PAM-(D)Ala-(D)Arg- (L)Leu-PAM-(D)Glu-(D)Arg-(L)Val-PAM-(D)Phe-(D)Lys-Gly-PAM. LC-MS (ESI): calculated: 5123.7 Da; found: 5123.0±0.8 Da.

FIG. 14I is a graph showing the LC-MS analysis of the synthetic compound IL6R-87-5 with the sequence of Fluorescein-(D)Arg-(D)Trp-(L)Phe-PAM-(D)Leu-(D)Arg-(L)Leu-PAM-(D)Asn-(D)Thr-(L)Leu-PAM-(D)Pro-(D)Arg-(L)Val-PAM-Gly-(D)Ala-(L)Val-PAM-(D)Glu-(D)Arg-(L)Leu-PAM-(D)Ala-(D)Arg-(L)Leu- PAM-(D)Pro-(D)Arg-(L)Ala-PAM-(D)Phe-(D)Arg-Gly-PAM. LC-MS (ESI): calculated: 4902.6 Da; found: 4902.7±0.9 Da.

FIG. 14J is a graph showing the LC-MS analysis of the synthetic compound IL6R-87-6 with the sequence of Fluorescein-(D)Arg-(D)Trp-(L)Phe-PAM-(D)Pro-(D)Asn-(L)Val-PAM-(D)Pro-(D)Lys-(L)Ala-PAM-(D)Trp-(D)Arg-(L)Leu-PAM-(D)Ala-(D)Lys-(L)Phe-PAM-(D)Pro-(D)Glu-(L)Phe-PAM-(D)Ala-(D)Arg- (L)Leu-PAM-(D)Trp-(D)Lys-(L)Leu-PAM-(D)Phe-(D)Lys-Gly-PAM. LC-MS (ESI): calculated: 5071.8 Da; found: 5071.5±0.4 Da.

FIG. 14K is a graph showing the LC-MS analysis of the synthetic compound IL6R-87-8 with the sequence of Fluorescein-(D)Asn-(D)Val-(L)Phe-PAM-(D)Tyr-(D)Ser-(L)Val-PAM-(D)Arg-(D)Ser-(L)Phe-PAM-(D)Phe-(D)Arg-(L)Ala-PAM-(D)Tyr-(D)Lys-(L)Ala-PAM-(D)Glu-(D)Arg-(L)Leu-PAM-(D)Ala-(D)Arg- (L)Leu-PAM-(D)Pro-(D)Arg-(L)Ala-PAM-(D)Phe-(D)Lys-Gly-PAM. LC-MS (ESI): calculated: 4931.5 Da; found: 4931.5±0.5 Da.

FIG. 14L is a graph showing the LC-MS analysis of the synthetic compound IL6R-87-10 with the sequence of Fluorescein-(D)Arg-(D)Trp-(L)Phe-PAM-(D)Leu-(D)Arg-(L)Leu-PAM-(D)Phe-(D)Glu-(L)Val-PAM-(D)Lys-(D)Glu-(L)Leu-PAM-(D)Ala-(D)Lys-(L)Phe-PAM-(D)Asn-(D)Arg-(L)Phe-PAM-(D)Ala-(D)Arg- (L)Leu-PAM-(D)Trp-(D)Lys-(L)Leu-PAM-(D)Ala-(D)Val-(L)Leu-PAM. LC-MS (ESI): calculated: 5132.4 Da; found: 5132.1±0.6 Da.

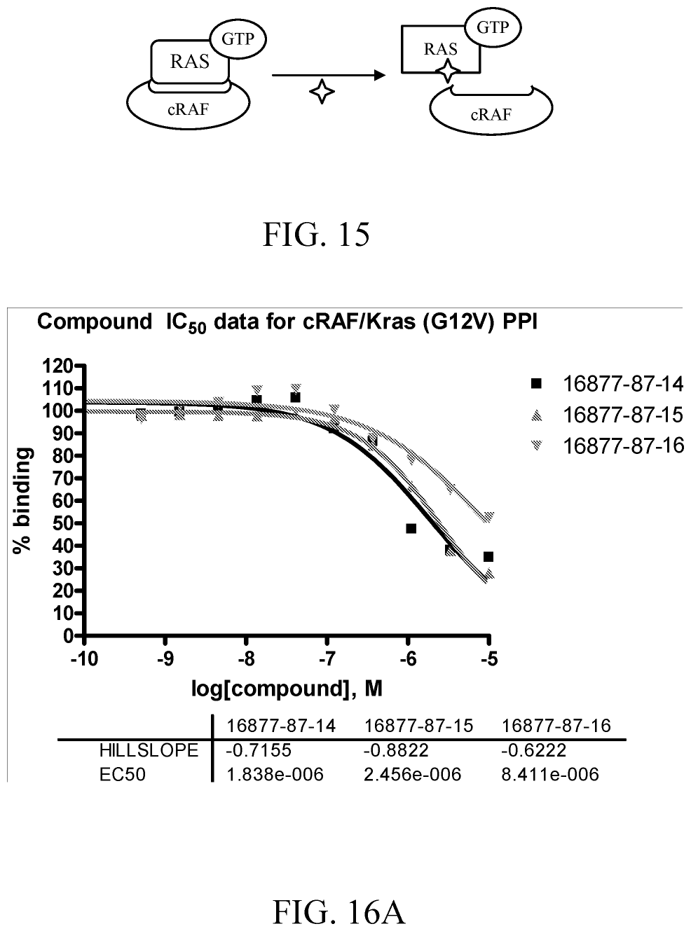

FIG. 15 illustrates an example experiment for K-Ras and Raf PPI profiling, in accordance with the present disclosure. In some experimental embodiments, K-Ras-cRaf homogenous time resolved fluorescence (HTRF)-based PPI profiling was conducted using five synthetic compounds from Table 13: KRAS-1-1, KRAS-1-6, KRAS-1-8, KRAS-1-9, and KRAS-1-13, but without the fluorescein at the C-terminus of the compounds. The five synthetic compounds were tested against one protein, K-Ras G12V (e.g., b-Kras G12V (GppNHp) (aa 2-169), at in 10-concentration IC50 mode with 3-fold serial dilution at a starting concentration of 10 μM. A control compound, BI-2852, which is a K-Ras inhibitor, was tested in 10-concentration IC50 mode with 3-fold serial dilution at a starting concentration of 50 μM. The compounds were pre-incubated with K-Ras G12V for 30 minutes at room temperature. The assay included a substrate with a plurality of wells, in which proteins of 30 nM K-Ras G12V and 10 nM Raf were used. In particular, GST-cRaf (aa 2-203) was used.

In some experimental embodiments, a buffer was added to the assay wells which included 20 mN HEPES pH 7.4, 150 mM NaCl, 5 mM MgCl2, 1 mM DTT, 0.005% NP40, and 1% DMSO. 5 uL of 3×K-Ras protein was then added to the assay wells, followed by the synthetic compounds which were added using acoustic technology (ECHO, Labcyte) followed by thirty minutes incubation at room temperature. Afterwards, 5 uL of 3×cRaf protein was added to the assay wells and 5 uL of detection mix was then added to the assay wells after 30 minutes. The detection mix included Mab anti-GST-Tb cryptate (Cisbio 61GSTTLB) and streptavidin-XL665 (Ex/Em=(337/665; 620) in PHERAstar (BMG Labtech)). HTRF signal was measured 60120 minutes later. Table 17 below provides a summary of the results.

TABLE 17

| | K-Ras G12V | | |
|---|---|---|---|
| % DMSO in | 1% | Results in | KD[nM] in MST |
| Reaction | Compound IC50 | Inhibition Assay in | (with fluorescein |
| Compound | (M) | MST | on N-term) |
| KRAS-1-1 | 1.84E−06 | | 24 |
| KRAS-1-6 | 2.46E−06 | | 21 |
| KRAS-1-8 | 8.41E−06 | Showed complete | 44 |
| | | inhibition | |
| KRAS-1-9 | 1.82E−06 | | 32 |
| KRAS-1-13 | 1.57E−06 | Showed partial | 30 |
| | | inhibition | |
| BI-2852 | 1.60E−06 | | |

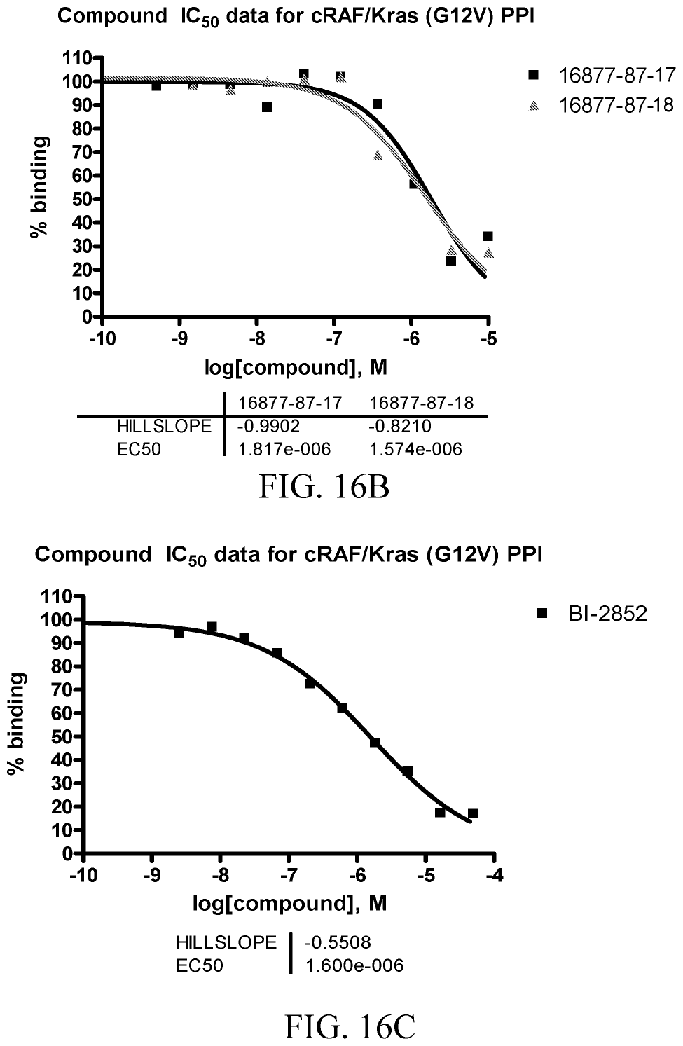

FIGS. 16A-16C illustrate example PPI profiling data for synthetic compounds, in accordance with the present disclosure. The data includes raw data (signal-background without cRaf protein but with all other component), % binding (relative to DMSO controls), and curve fits. Curve fits were performed when the activities at the highest concentration of the synthetic compounds were less than 65%. Tables 18A-18B below includes the raw data (e.g., blank corrected HTRF) and Tables 19A-19B includes the % binding.

TABLE 18A
Raw Data for Synthetic Compounds

| | Compound | KRAS- | KRAS-1- | KRAS-1- | KRAS-1- | KRAS-1- |
|---|---|---|---|---|---|---|
| | ID | 1-1 | 6 | 8 | 9 | 13 |
| blank | 1.00E−05 | 4849 | 3880 | 7296 | 4707 | 3798 |
| corrected | | | | | | |
| HTRF | | | | | | |
| | 3.33E−06 | 5282 | 5253 | 9028 | 3269 | 3957 |
| | 1.11E−06 | 6589 | 9200 | 10835 | 7790 | 8379 |
| | 3.70E−07 | 11981 | 11760 | 12171 | 12513 | 9542 |
| | 1.23E−07 | 12756 | 13441 | 13924 | 14122 | 14147 |
| | 4.12E−08 | 14675 | 13672 | 15230 | 14333 | 14025 |
| | 1.37E−08 | 14500 | 13561 | 15117 | 12329 | 13885 |
| | 4.57E−09 | 14067 | 13584 | 14396 | 13688 | 13413 |
| | 1.52E−09 | 13807 | 13652 | 14168 | 13613 | 13665 |
| | 5.08E−10 | 13672 | 13595 | 13397 | 13596 | 5044 |
| | DMSO | 14163 | 13860 | 14062 | 13823 | 13827 |

TABLE 18B
Raw Data for BI-2852

| | Compound | |
|---|---|---|
| | ID | BI-2852 |
| blank | 5.00E−05 | 2344 |
| corrected | | |
| HTRF | | |
| | 1.67E−05 | 2409 |
| | 5.56E−06 | 4849 |
| | 1.85E−06 | 6562 |
| | 6.17E−07 | 8628 |
| | 2.06E−07 | 10050 |
| | 6.86E−08 | 11886 |
| | 2.29E−08 | 12784 |
| | 7.62E−09 | 13422 |
| | 2.54E−09 | 13013 |
| | DMSO | 13602 |

TABLE 19A
% Binding for Synthetic Compounds

| Compound | KRAS- | KRAS-1- | KRAS-1- | KRAS-1- | KRAS-1- |
|---|---|---|---|---|---|
| ID | 1-1 | 6 | 8 | 9 | 13 |
| 1.00E−05 | 34.94* | 27.96* | 52.58* | 33.92** | 27.37** |
| 3.33E−06 | 38.06* | 37.86* | 65.06* | 23.56* | 28.52* |
| 1.11E−06 | 47.48 | 66.30 | 78.08 | 56.14 | 60.38 |
| 3.70E−07 | 86.34 | 84.75 | 87.71 | 90.18 | 68.76 |
| 1.23E−07 | 91.93 | 96.86 | 100.35 | 101.78 | 101.95 |
| 4.12E−08 | 105.75 | 98.53 | 109.56 | 103.29 | 101.08 |
| 1.37E−08 | 104.50 | 97.73 | 108.95 | 88.85 | 100.06 |
| 4.57E−09 | 101.38 | 97.90 | 103.74 | 98.65 | 96.67 |
| 1.52E−09 | 99.50 | 98.39 | 102.11 | 98.10 | 98.48 |
| 5.08E−10 | 98.53 | 97.97 | 96.55 | 97.95 | 36.35° |
| DMSO | 102.07 | 99.88 | 101.34 | 99.62 | 99.5 |
| HillSlope | −0.7155 | −0.8822 | −0.6222 | −0.9902 | −0.821 |
| IC50(M) | 1.84E−06 | 2.46E−06 | 8.41E−06 | 1.82E−06 | 1.57E−06 |

*indicates potential interference
°indicates excluded from fit
**indicates potential interference/aggregation

TABLE 19B
% Binding for BI-2852

| Compound | |
|---|---|
| ID | BI-2852 |
| 5.00E−05 | 16.9 |
| 1.67E−05 | 17.36 |
| 5.56E−06 | 34.94 |
| 1.85E−06 | 47.29 |
| 6.17E−07 | 62.18 |
| 2.06E−07 | 72.42 |
| 6.86E−08 | 85.66 |
| 2.29E−08 | 92.13 |
| 7.62E−09 | 96.73 |
| 2.54E−09 | 93.78 |
| DMSO | 98.03 |
| HillSlope | −0.5508 |
| IC50(M) | 1.60E−06 |

More particularly, FIG. 16A illustrates a graph of the compound IC50 data for cRaf/K-Ras (G12V) PPI for the synthetic compounds KRAS-1-1, KRAS-1-6, and KRAS=1-8. FIG. 16B illustrates a graph of the compound IC50 data for cRaf/K-Ras (G12V) PPI for the synthetic compounds KRAS-1-9 and KRAS-1-13. FIG. 16C illustrates a graph of the compound IC50 data for cRaf/K-Ras (G12V) PPI for the BI-2852 compounds.

Table 20 below provides an interference check on the synthetic compounds using biotinylated GST, which was used with the detection mix to check for compound effect on fluorescence. If no effect is present, comparable signals are expected in the wells.

TABLE 20
Interference

| | [Assay | | 665 nm | 615 nm | | | |
|---|---|---|---|---|---|---|---|
| Compound | Conc.], μM | HTRF | emis. | emis. | | | |
| No B-GST | 0 | 656.15 | 10128 | 154354 | | | |
| No B-GST | 0 | 650.18 | 10067 | 154833 | | | |
| No B-GST | 0 | 655.45 | 10072 | 153666 | | | |
| No B-GST | 0 | 653.85 | 11465 | 175345 | | | |
| No B-GST | 0 | 652.27 | 10032 | 153801 | | | |
| No B-GST | 0 | 657.01 | 10274 | 156376 | | | |
| No B-GST | 0 | 663.98 | 10281 | 154838 | | | |
| No B-GST | 0 | 645.37 | 10469 | 162218 | | | |
| No B-GST | 0 | 653.38 | 10072 | 154153 | | | |
| No B-GST | 0 | 655.28 | 10302 | 154857 | | | |
| No B-GST | 0 | 666.95 | 10344 | 155095 | | | |
| No B-GST | 0 | 653.36 | 10024 | 153422 | DMSO | 14279.34556 | % signal |
| | | | | | av | | compared |
| | | | | | | | to DMSO |
| KRAS-1-1 | DMSO | 14647.88 | 204004 | 139272 | Sub no | 13623.34556 | 102.705168 |
| | | | | | GST | | |
| KRAS-1-1 | 10 | 9487.28 | 145219 | 153067 | | | 64.8246054* |
| KRAS-1-1 | 3.33333333333333 | 10114.07 | 139381 | 137809 | | | 69.4254577* |
| KRAS-1-1 | 1.11111111111111 | 17987.14 | 173997 | 145153 | | | 83.1744299 |
| KRAS-1-1 | 0.37037037037037 | 13676.98 | 190493 | 139280 | | | 95.5784315 |
| KRAS-1-1 | 0.123456790123457 | 14455.69 | 199198 | 137799 | | | 101.294428 |
| KRAS-1-1 | 0.0411522633744856 | 14136.83 | 206296 | 145928 | | | 98.9538872 |
| KRAS-1-1 | 0.0137174211248285 | 13344.65 | 187954 | 140846 | | | 93.139016 |
| KRAS-1-1 | 0.00457247370827618 | 14227.74 | 209788 | 147450 | | | 99.6211976 |
| KRAS-1-1 | 0.00152415790275873 | 14704.3 | 204553 | 139111 | | | 103.11931 |
| KRAS-1-1 | 0.000508052634252908 | 14760.33 | 202311 | 137064 | | | 103.53059 |
| KRAS-1-1 | DMSO | 14084.02 | 203162 | 144250 | | | 98.5662438 |
| KRAS-1-6 | DMSO | 14621.14 | 197979 | 135406 | | | 102.508888 |
| KRAS-1-6 | 19 | 9106.12 | 108708 | 119379 | | | 62.0267611* |
| KRAS-1-6 | 3.333333333 | 10798.77 | 137301 | 127145 | | | 74.451389 |
| KRAS-1-6 | 1.111111111 | 12896.78 | 177728 | 137808 | | | 89.8514976 |
| KRAS-1-6 | 0.37037037 | 14380.1 | 193536 | 134586 | | | 100.739572 |
| KRAS-1-6 | 0.12345679 | 14326.15 | 197516 | 137871 | | | 100.343561 |
| KRAS-1-6 | 0.041152263 | 14676.28 | 198372 | 135165 | | | 102.913634 |
| KRAS-1-6 | 0.013717421 | 14615.66 | 195559 | 133801 | | | 102.468663 |
| KRAS-1-6 | 0.004572474 | 14604.79 | 193972 | 132814 | | | 102.38887 |
| KRAS-1-6 | 0.001524158 | 14648.3 | 195382 | 133382 | | | 102.70825 |
| KRAS-1-6 | 0.000508053 | 14704.98 | 194788 | 132464 | | | 103.12443 |
| KRAS-1-6 | DMSO | 14491.65 | 196643 | 135694 | | | 101.55839 |
| KRAS-1-8 | DMSO | 14389.37 | 191275 | 132928 | | | 100.80762 |
| KRAS-1-8 | 10 | 8346.72 | 161247 | 193186 | | | 56.452506* |
| KRAS-1-8 | 3.333333333 | 9992.21 | 169410 | 169542 | | | 68.530964 |
| KRAS-1-8 | 1.111111111 | 11810.88 | 170418 | 144289 | | | 81.880621 |
| KRAS-1-8 | 0.37037037 | 13456.73 | 186038 | 138249 | | | 93.951721 |
| KRAS-1-8 | 0.12345679 | 13942.77 | 192512 | 138073 | | | 97.529421 |
| KRAS-1-8 | 0.041152263 | 12602.68 | 203474 | 161453 | | | 87.692703 |
| KRAS-1-8 | 0.013717421 | 14052.13 | 198610 | 141338 | | | 98.33216 |
| KRAS-1-8 | 0.004572474 | 14691.02 | 198749 | 135286 | | | 103.02183 |
| KRAS-1-8 | 0.001524158 | 14599.8 | 198931 | 136256 | | | 102.35224 |
| KRAS-1-8 | 0.000508053 | 14270.77 | 197095 | 138111 | | | 99.937052 |
| KRAS-1-8 | DMSO | 14743.79 | 199308 | 135181 | | | 103.40918 |
| KRAS-1-9 | DMSO | 14645.48 | 203168 | 138724 | | | 102.68755 |
| KRAS-1-9 | 10 | 7969.96 | 108766 | 136470 | | | 53.686959* |
| KRAS-1-9 | 3.333333333 | 10136.62 | 150983 | 148948 | | | 69.590982 |
| KRAS-1-9 | 1.111111111 | 13246.01 | 182791 | 137997 | | | 92.414965 |
| KRAS-1-9 | 0.37037037 | 14252.32 | 199762 | 140161 | | | 99.801623 |
| KRAS-1-9 | 0.12345679 | 14485.06 | 197905 | 136627 | | | 101.51001 |
| KRAS-1-9 | 0.041152263 | 14666.68 | 203487 | 138741 | | | 102.84317 |
| KRAS-1-9 | 0.013717421 | 14471.26 | 196566 | 135832 | | | 101.40872 |
| KRAS-1-9 | 0.004572474 | 14310.62 | 197790 | 138212 | | | 100.22957 |
| KRAS-1-9 | 0.001524158 | 14663.3 | 197632 | 134780 | | | 102.81836 |
| KRAS-1-9 | 0.000508053 | 14241 | 200190 | 140573 | | | 99.718531 |
| KRAS-1-9 | DMSO | 14083.98 | 197918 | 140527 | | | 98.56595 |
| KRAS-1-13 | DMSO | 14630.41 | 186924 | 127764 | | | 102.57693 |
| KRAS-1-13 | 10 | 9154.1 | 120088 | 131185 | | | 62.378951* |
| KRAS-1-13 | 3.333333333 | 11612.52 | 150223 | 129363 | | | 80.424591 |
| KRAS-1-13 | 1.111111111 | 13534.47 | 185004 | 136691 | | | 94.53236 |
| KRAS-1-13 | 0.37037037 | 14335.41 | 196160 | 136836 | | | 100.41153 |
| KRAS-1-13 | 0.12345679 | 14331 | 191604 | 133699 | | | 100.32916 |
| KRAS-1-13 | 0.041152263 | 14567.32 | 197648 | 135679 | | | 102.11383 |
| KRAS-1-13 | 0.013717421 | 14540.23 | 196597 | 135209 | | | 101.91498 |
| KRAS-1-13 | 0.004572474 | 14654.59 | 195016 | 133075 | | | 102.75442 |
| KRAS-1-13 | 0.001524158 | 14706.5 | 195445 | 132897 | | | 103.13546 |
| KRAS-1-13 | 0.000508053 | 14527.21 | 197480 | 135938 | | | 101.81941 |
| KRAS- 1-13 | DMSO | 12797.53 | 195957 | 153121 | | | 89.122969 |
| | | | | | | | 0 |

Various embodiments are implemented in accordance with the underlying Provisional Application Ser. No. 63/092,341, entitled “High Affinity Non-Natural Ligands Against Protein Targets,” filed Oct. 15, 2020, and including the Appendix to the Specification, and Provisional Application Ser. No. 63/093,072, entitled “High Affinity Non-Natural Ligands Against Protein Targets,” filed Oct. 16, 2020, and including the Appendix to the Specification, to which benefit is claimed and which are fully incorporated herein by reference for their general and specific teachings. For instance, embodiments herein and/or in the Provisional Applications can be combined in varying degrees (including wholly). Reference can also be made to the experimental teachings and underlying references provided in the underlying Provisional Applications. Embodiments discussed in the Provisional Applications are not intended, in any way, to be limiting to the overall technical disclosure, or to any part of the claimed disclosure unless specifically noted.

Although specific examples have been illustrated and described herein, a variety of alternate and/or equivalent implementations can be substituted for the specific examples shown and described without departing from the scope of the present disclosure. This application is intended to cover any adaptations or variations of the specific examples discussed herein.

1. A competitive binding assay, the assay comprising:
a plurality of sub-regions;
a plurality of synthetic compounds on beads, wherein each of the plurality of sub-region includes one of the plurality of synthetic compounds;
a biological target labeled with a first detectable label in each of the plurality sub-regions; and
a biological counter target labeled with a second detectable label in each of the plurality of sub-regions, wherein the biological counter target is configured to bind to the biological target when the biological target is in a first orientation, and a first subset of the plurality of synthetic compounds bind to the biological target and effect interactions between the biological target and the biological counter target.

2. The assay of claim 1, wherein the first subset of the plurality of synthetic compounds are to bind the biological target in a different orientation than the first orientation and inhibit interactions between the biological target and the biological counter target.

3. The assay of claim 2, wherein a second subset of the plurality of synthetic compounds bind to the biological target in the first orientation and a third subset of the plurality of synthetic compounds bind to the biological counter target.

4. The assay of claim 1, wherein the first subset of the plurality of synthetic compounds bind to the biological target such that the biological target is in the first orientation and permit for interactions between the biological target and the biological counter target.

5. The assay of claim 1, wherein respective sub-regions associated with the first subset of the plurality of synthetic compounds provide a first fluorescent signal associated with the first detectable label and not a second fluorescent signal associated with the second detectable label.

6. The assay of claim 1, wherein respective sub-regions associated with the first subset of the plurality of synthetic compounds provide a first fluorescent signal associated with the first detectable label and provide a second fluorescent signal associated with the second detectable label.

7. The assay of claim 1, wherein the plurality of synthetic compounds include:
different subsets of a plurality of molecules, each of the plurality of molecules including a plurality of subgroups and exhibiting a mass spectrometry characteristic that is distinguishable from mass spectrometry characteristics of other molecules of the plurality; and
cleavable groups linking at least some of the different subsets of the plurality of molecules and the bead to facilitate mass-spectroscopy based sequencing of the plurality of synthetic compounds.

8. The assay of claim 1, wherein the first detectable label is different from the second detectable label, and the biological target and biological counter target are proteins or nucleic acids.

9. An apparatus, comprising:
a binding assay comprising:
a plurality of sub-regions;
a plurality of synthetic compounds on beads that are distinguishable by mass spectrometry, wherein each of the plurality of sub-regions includes one of the plurality of synthetic compounds;
a biological target labeled with a first detectable label in each of the plurality of sub-regions; and
a biological counter target labeled with a second detectable label in each of the plurality of sub-regions, the biological counter target configured to bind to the biological target when the biological target is in a first orientation; and
scanning circuitry to identify a first subset of the plurality of synthetic compounds that bind to the biological target and that effects interactions between the biological target and the biological counter target.

10. The apparatus of claim 9, wherein the scanning circuitry is to provide a qualitative measure of binding affinity based a detected level of a signal associated with at least one of the first detectable label and the second detectable label.

11. The apparatus of claim 9, wherein the scanning circuitry is to provide a ratio of the biological target binding to the biological counter target binding based on detection of a first level of a signal associated with the first detectable label and a second level of a signal associated with the second detectable label.

12. The apparatus of claim 9, further including cell picking circuitry to isolate the first subset of the plurality of synthetic compounds.

13. The apparatus of claim 9, wherein the plurality of synthetic compounds include:
different subsets of a plurality of molecules, each of the plurality of molecules including a plurality of subgroups and exhibiting a mass spectrometry characteristic that is distinguishable from mass spectrometry characteristics of other molecules of the plurality; and
cleavable groups linking at least some of the different subsets of the plurality of molecules and the bead.

14. The apparatus of claim 13, further including mass spectrometry circuitry to sequence the first subset of the plurality of synthetic compounds by identifying the mass spectrometry characteristics of the respective subsets of molecules of the first subset of the plurality of synthetic compounds.

15. The apparatus of claim 9, wherein the scanning circuitry is to identify the first subset of the plurality of synthetic compounds based on:
a first fluorescent signal associated with the first detectable label; and
a second fluorescent signal associated with the second detectable label.

16. A method, comprising:
exposing a plurality of sub-regions of a binding assay to a biological target labeled with a first detectable label and a biological counter target labeled with a second detectable label,
wherein each of the plurality of sub-regions include one of a plurality of synthetic compounds on a bead that are distinguishable by mass spectrometry, wherein the biological counter target is configured to bind to the biological target when the biological target is in a first orientation; and
detecting binding of a first subset of the plurality of synthetic compounds to the biological target that effects interactions between the biological target and the biological counter target by identifying signals associated with the first detectable label and the second detectable label.

17. The method of claim 16, wherein detecting the binding of the first subset of the plurality of synthetic compounds includes identifying blocking of binding between the biological counter target and the biological target by identifying the signals associated with the second detectable label are below a threshold.

18. The method of claim 16, further including isolating the first subset of the plurality of synthetic compounds and performing mass spectrometry to identify sequences of the first subset of the plurality of synthetic compounds.

19. The method of claim 16, wherein exposing the assay to the biological target and the biological counter target includes:
incubating the plurality of synthetic compounds with the biological target and the biological counter target; and
after incubating, washing unbound biological targets and biological counter targets from the assay.

20. The method of claim 16, wherein the plurality of synthetic compounds include:
different subsets of a plurality of molecules, each of the plurality of molecules including a plurality of subgroups and exhibiting a mass spectrometry characteristic that is distinguishable from mass spectrometry characteristics of other molecules of the plurality; and
cleavable groups linking at least some of the different subsets of the plurality of molecules and the bead, and
the method further includes:
separating the first subset of the plurality of synthetic compounds from the beads and the respective different subset of molecules forming the synthetic compounds of the first subset from one another; and
sequencing the first subset of the plurality of synthetic compounds by identifying the mass spectrometry characteristics of the respective different subset of molecules via mass spectrometry.
